(19) United States
(12) Patent Application Publication
(10) Pub. No.: US 2012/0115925 A1
(43) Pub. Date: May 10, 2012

(54) Allelic Variants Associated with Advanced Age-Related Macular Degeneration

(75) Inventors: Margaret M. DeAngelis, Bountiful, UT (US); Margaux A. Morrison, Boston, MA (US)

(73) Assignee: Massachusetts Eye and Ear Infirmary, Boston, MA (US)

(21) Appl. No.: 13/141,898

(22) PCT Filed: Dec. 23, 2009
(86) PCT No.: PCT/US2009/069423

§ 371 Date: Jan. 9, 2012

Related U.S. Application Data
(60) Provisional application No. 61/203,566, filed on Dec. 23, 2008.

(51) Int. Cl. C12Q 1/68; A61K 31/7088; A61K 31/713; G01N 33/566

(52) U.S. Cl. 514/44 A; 436/501; 514/44 R; 435/6.11

(57) ABSTRACT

The invention relates to allelic variants and haplotypes of the Complement Factor H (CFH) gene, the Complement Component 3 (C3) gene, and the Complement Factor D (CFD) gene, associated with either an elevated or a reduced risk that an individual will develop age-related macular degeneration (AMD), methods of diagnosing such risk in an individual based on the presence or absence of such variants, and methods and reagents for diagnosis and treatment of AMD.

### REFERENCE TO RELATED APPLICATION

This application claims benefit of the filing date of U.S. provisional application Ser. No. 61/203,566, filed Dec. 23, 2008, which is hereby incorporated by reference.

### STATEMENT REGARDING FEDERALLY SPONSORED RESEARCH

The work described in this application was sponsored in part by the National Eye Institute under Grant No. EY-014458-06. The United States Government may have certain rights in the invention.

### FIELD OF THE INVENTION

The invention relates generally to methods and compositions for determining whether an individual is at risk of developing age-related macular degeneration by detecting whether the individual has one or more of a set of risk variants and haplotypes associated with the Complement Factor H (“CFH”) gene.

### BACKGROUND OF THE INVENTION

Advanced age-related macular degeneration is the most common cause of legal blindness in the United States. This loss of central vision adversely affects one's every day life by impairing the ability to read, drive, and recognize faces. It is estimated that 1.75 million U.S. citizens have advanced AMD in at least one eye. As delivery of health care improves and the population lives longer, this number will likely increase to 3 million individuals by the year 2020. See, Eye Diseases Prevalence Research Group, “Prevalence of Age-Related Macular Degeneration”, Archives of Ophthalmology, 122:564-574 (2004).

As humans age, the retina typically undergoes changes that are visible ophthalmoscopically, including the development of drusen, atrophic patches of retinal pigment epithelium (RPE), and hyperpigmented or clumped RPE. These changes can occur throughout the retina but they preferentially involve the macula. The age at which these changes first become evident varies from person to person, but they are frequently seen in those over age 40. When there is associated loss in central vision, the term “age-related macular degeneration” (AMD) is often used, although some investigators use the term AMD for these aging changes even if the visual acuity is normal. “Age-related maculopathy” is another term used interchangeably with AMD. The degree of visual loss can be mild (e.g., 20/30) or can be so severe as to cause legal blindness (20/200 or worse). Visual loss is due to degeneration of the photoreceptor cells in the macula.

A minority of cases have what is termed “late”, or “advanced”, AMD. Two forms of advanced AMD are recognized clinically: geographic atrophy AMD and neovascular, or wet, AMD. Geographic atrophy is characterized by a slow progressive degeneration of the retinal pigment epithelium (RPE), resulting in the gradual loss of photoreceptors. Geographic atrophy represents an area of loss of the RPE and choriocapillaris, and the photoreceptors overlying the patch are absent or dysfunctional. If the geographic atrophy involves the fovea, there is severely reduced visual acuity.

It is the wet or neovascular form of advanced AMD that is responsible for the majority of debilitating vision loss due to AMD. Neovascular AMD is characterized by growth of new abnormal blood vessels from beneath the retina that can cause severe and rapid vision loss due to hemorrhage and exudation. The process whereby blood vessels grow from the adjacent choroid beneath the outer retina is referred to as choroidal neovascularization. These new blood vessels can bleed and leak fluid, resulting in sudden visual loss, and eventually may form a scar with associated fibrous and glial tissue (called a disciform scar) that also invariably results in visual loss due to the degeneration of the overlying photoreceptor cells. If the region of the macula affected includes the fovea, which is typical, there is marked loss of visual acuity. The majority of eyes with neovascular AMD ultimately lose vision to levels of 20/200 or worse without treatment. Jager, R. D., et al., “Age-related macular degeneration”, N. Engl. J. Med., 358:2606-17 (2008).

Funduscopically visible age-related macular changes other than neovascularization generally are comparable between the two eyes of an individual. Barondes, M. et al., “Bilaterality of drusen”, Br. J. Ophthalmol. 74:180-182, 1990. Neovascular AMD initially affects only one eye, but patients with neovascular AMD in only one eye often have severe aging changes of the macula in the fellow eye, such as numerous or large drusen or extensive pigment abnormalities. Furthermore, individuals with neovascular AMD in one eye are at greatly elevated risk for new blood vessels growing in the fellow eye; between four and thirteen percent of such fellow eyes will develop neovascularization per year based on retrospective and prospective studies. Bressler, S. B. et al., “Natural course of choroidal neovascular membranes within the foveal avascular zone in senile macular degeneration”, Am. J. Ophthalmol., 93:157-163 (1982); Strahlman, E. R. et al., “The second eye of patients with senile macular degeneration”, Arch. Ophthalmol., 101:1191-93 (1983); Macular Photocoagulation Study Group, “Risk factors for choroidal neovascularization in the second eye of patients with juxtafoveal or subfoveal choroidal neovascularization secondary to age-related macular degeneration”, Arch. Ophthalmol., 115:741-47 (1997); Sandberg, M. A. et al., “High-risk characteristics of fellow eyes of patients with unilateral neovascular age-related macular degeneration”, Ophthalmology, 105:441-47 (1998).

Treatment of the wet form of age-related macular degeneration has proved difficult. Several methods have been approved in the United States of America for treating the wet form of age-related macular degeneration. Two are laser based approaches, and include laser photocoagulation and photodynamic therapy using a benzoporphyrin derivative photosensitizer known as Visudyne® (Novartis AG). Two require the administration of therapeutic molecules that bind and inactivate or reduce the activity of Vascular Endothelial Growth Factor (VEGF), one is known as Lucentis®(Genentech, San Francisco Calif.), which is a humanized anti-VEGF antibody fragment having the generic name ranibizumab. The other is known as Macugen® (Eyetech, Inc., NY, N.Y.), which is an anti-VEGF aptamer generically referred to as pegaptanib sodium injection.

During laser photocoagulation, thermal laser light is used to heat and photocoagulate the neovasculature of the choroid. A problem associated with this approach is that the laser light must pass through the photoreceptor cells of the retina in order to photocoagulate the blood vessels in the underlying choroid. As a result, this treatment destroys the photoreceptor cells of the retina creating blind spots with associated vision loss.

During photodynamic therapy, a benzoporphyrin derivative photosensitizer known as Visudyne® (available from QLT, Inc., Vancouver, Canada) is administered to the individual to be treated. Once the photosensitizer accumulates in the choroidal neovasculature, non-thermal light from a laser is applied to the region to be treated, which activates the photosensitizer in that region. The activated photosensitizer generates free radicals that damage the vasculature in the vicinity of the photosensitizer (see, U.S. Pat. Nos. 5,798,349 and 6,225,303). This approach is more selective than laser photocoagulation and is less likely to result in blind spots. Under certain circumstances, this treatment has been found to restore vision in patients afflicted with the disorder (see, U.S. Pat. Nos. 5,756,541 and 5,910,510).

Lucentis, which is available from Genentech, Inc., CA, is a humanized therapeutic antibody that binds and inhibits or reduces the activity of VEGF, a protein believed to playa role in angiogenesis. Pegaptanib sodium, which is available from OSI Pharmaceuticals, Inc., NY, is a pegylated aptamer that targets VEGFI65, the isoform believed to be responsible for primary pathological ocular neovascularization.

Despite these efforts, there is still an ongoing need for methods of identifying individuals at risk of developing age-related macular degeneration so that such individuals can be monitored more closely and then treated, so as to slow, stop or reverse the onset of age-related macular degeneration. In addition, an ongoing need for new methods of preventing the onset of age-related macular degeneration, and, once established, the treatment of age-related macular degeneration.

### BRIEF SUMMARY OF THE INVENTION

In a first aspect is a method of determining an individual's susceptibility to age-related macular degeneration (AMD), which includes testing a biological sample obtained from the individual for the presence or absence of an allelic variant of the Complement Factor H(CFH) gene, wherein the allelic variant is a guanine at polymorphic site rs482934, and wherein the presence of the allelic variant indicates that the individual has a significant risk for developing AMD. The individual can be homozygous for the allelic variant, or can be heterozygous for the allelic variant.

In the methods set forth above, an individual's susceptibility to age-related macular degeneration (AMD) is understood to mean whether an individual human subject has a propensity to develop AMD, or in other words is more or less likely to develop AMD than other individuals in the population as a whole. The terms “diagnose” and “diagnosis” refer to the ability to determine whether an individual has the propensity to develop disease (including with or without signs or symptoms). Diagnosis of propensity to develop disease can also be called “screening” and, as used herein, the terms diagnosis and screening are used interchangeably. It will be appreciated that having an increased or decreased propensity to developing a condition refers to the likelihood of developing the condition relative to individuals in the population without the condition.

In another aspect is a method of determining an individual's susceptibility to age-related macular degeneration (AMD), which includes testing a biological sample obtained from the individual for the presence or absence of an allelic variant of the CFH gene, wherein the allelic variant is a cytosine at polymorphic position rs375046, and wherein the presence of the allelic variant indicates that the individual has a significant risk for developing AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of an allelic variant of the CFH gene, wherein the allelic variant is a cytosine at polymorphic site rs16840522 and wherein the presence of the allelic variant indicates that the individual has a significantly reduced susceptibility to AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of an allelic variant of the Complement Factor D (CFD) gene, wherein the allelic variant is a guanine at polymorphic site rs1683564, and wherein the presence of the allelic variant indicates that the individual has a reduced susceptibility to AMD. The individual can be homozygous for the allelic variant, or can be heterozygous for the allelic variant.

In yet another aspect is a method of determining an individual's susceptibility to age-related macular degeneration (AMD), which includes testing a biological sample obtained from the individual for the presence or absence of a polymorphic haplotype, wherein the haplotype comprises a guanine at polymorphic site rs800292 and a common allele of no insertion at polymorphic site rs35507625, and wherein the presence of the allelic variant indicates that the individual has an increased susceptibility to AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of a polymorphic haplotype, wherein the haplotype comprises an adenine at polymorphic site rs800292 and a thymine-thymine double nucleotide insertion at polymorphic site rs35507625, and wherein the presence of the haplotype indicates that the individual has a reduced susceptibility to AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of a polymorphic haplotype, wherein the haplotype comprises a nucleic acid segment which includes an adenine at polymorphic site rs572515, and wherein the presence of the haplotype indicates that the individual has an increased susceptibility to developing AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of a polymorphic haplotype, wherein the haplotype comprises an adenine at polymorphic site rs572515, an adenine at polymorphic site rs1061147, a thymine at polymorphic site rs7529589, a guanine at polymorphic site rs482934, a cytosine at polymorphic site rs1061170, a guanine at polymorphic site rs12038333, a guanine at polymorphic sited rs2274700, a guanine at polymorphic site rs203674, an adenine at polymorphic site rs3753396, a cytosine at polymorphic site rs375046, and a guanine at polymorphic site rs1065489, and wherein the presence of the haplotype indicates that the individual has an increased susceptibility to AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of a polymorphic haplotype, wherein the haplotype comprises an guanine at polymorphic site rs572515, a cytosine at polymorphic site rs1061147, a cytosine at polymorphic site rs7529589, a thymine at polymorphic site rs482934, a thymine at polymorphic site rs1061170, an adenine at polymorphic site rs12038333, an adenine at polymorphic sited rs2274700, a thymine at polymorphic site rs203674, an adenine at polymorphic site rs3753396, an adenine at polymorphic site rs375046, and a guanine at polymorphic site rs1065489, and wherein the presence of the haplotype indicates that the individual has a reduced susceptibility to AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of a polymorphic haplotype, wherein the haplotype comprises an guanine at polymorphic site rs572515, a cytosine at polymorphic site rs1061147, a cytosine at polymorphic site rs7529589, a thymine at polymorphic site rs482934, a thymine at polymorphic site rs1061170, an adenine at polymorphic site rs12038333, a guanine at polymorphic sited rs2274700, a thymine at polymorphic site rs203674, a guanine at polymorphic site rs3753396, an adenine at polymorphic site rs375046, and a guanine at polymorphic site rs1065489, and wherein the presence of the haplotype indicates that the individual has a reduced susceptibility to AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of a polymorphic haplotype, wherein the haplotype comprises a thymine at polymorphic site rs16840522, a cytosine at polymorphic site rs513699, a cytosine at polymorphic site rs425757, and a cytosine at polymorphic site rs410232, and wherein the presence of the haplotype indicates that the individual has an increased susceptibility to developing AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of a polymorphic haplotype, wherein the haplotype comprises a cytosine at polymorphic site rs16840522, a thymine at polymorphic site rs513699, a thymine at polymorphic site rs425757, and a guanine at polymorphic site rs410232, and wherein the presence of the haplotype indicates that the individual has a decreased susceptibility to AMD. The individual can be homozygous for the allelic variant, or can be heterozygous for the allelic variant.

In another aspect is a method of determining an individual's susceptibility to age-related macular degeneration (AMD), which includes testing a biological sample obtained from the individual for the presence or absence of a polymorphic haplotype, wherein the haplotype comprises a cytosine at polymorphic site rs7951, a guanine at polymorphic site rs2277984, an adenine at polymorphic site rs344555, a cytosine at polymorphic site rs11569565, and a cytosine at polymorphic site rs17030. The individual can be homozygous for the allelic variant, or can be heterozygous for the allelic variant.

The methods set forth above can be used as diagnostic methods, for determining a subject's propensity to develop age-related macular degeneration (AMD), comprising detecting the presence or absence of a allelic variants or haplotypes set forth herein. The methods may include obtaining the DNA from an individual and analyzing the DNA from the individual to determine whether the DNA contains the polymorphism of interest. As detailed below, certain polymorphisms indicate the individual has an increased susceptibility to developing AMD relative to a control population, while other polymorphisms indicate the individual has a reduced likelihood of developing AMD.

By way of example, any one of the methods above can include (i) combining the biological sample with one or more probes, wherein each of said one or more probes is characterized by its ability to bind with specificity to an allelic variant in the sample, and (ii) observing the presence or absence of binding between the probe and a macromolecule within the biological sample.

The probe can be, e.g., an oligonucleotide or an oligonucleotide derivative. By way of further example, the probe can be an oligonucleotide capable of priming polynucleotide synthesis in a polymerase chain reaction.

In one embodiment, a method of diagnosing an increased or reduced susceptibility to developing age-related macular degeneration (AMD) in a subject involves obtaining a sample of DNA from the subject and detecting in the DNA of the patient the presence or absence of a polymorphism associated with development of AMD, the presence of the polymorphism being an indication that the subject has an increased propensity to develop AMD and the absence of the polymorphism being an indication that the subject has a reduced propensity to develop AMD.

In a related aspect, a method of diagnosing an increased or reduced susceptibility to developing AMD can involve determining an individual's haplotype at the CFH or C3 locus. The methods include obtaining the DNA from an individual and analyzing the DNA of the individual to determine whether the individual possesses a segment of DNA that bears each of the allelic variants that make up one or more of the haplotypes characterized herein. As set forth more fully in the following text, certain haplotypes (“risk haplotypes”) indicate the individual has an increased susceptibility to develop AMD. Other haplotypes (“protective haplotypes”) indicate the individual has a decreased, or reduced, susceptibility to develop AMD. Yet again certain other haplotypes (“neutral haplotypes”, identified as “n/a”) indicate the individual has neither an increased nor a reduced likelihood of developing AMD.

In a related embodiment, the presence or absence of a variation at one of the polymorphic sites described herein is determined by analysis of a gene product, such as an RNA or a protein (e.g., protein isoform) encoded by the gene to which the allelic variant relates. Expression of a variant protein is an indication of a variation in the corresponding gene, and can indicate an increased or reduced propensity to develop AMD. Proteins can be detected using immunoassays and other methods.

In another embodiment, the methods of determining an individuals' susceptibility to AMD summarized above can be performed with the use of a probe that is an antibody or an antibody derivative. Methods of diagnosing susceptibility to developing AMD or other diseases by detecting a polymorphic variant in a biological sample of an individual involve use of an antibody-based assay, by contacting a biological sample, e.g., a serum sample, of the individual with the antibody that binds with specificity to a macromolecule exhibiting the polymorphism and detecting the presence or absence of the polymorphic variant in the macromolecule. In an embodiment, the antibody specifically interacts with an epitope specific to a variant Factor H polypeptide (i.e., not found in the wild-type Factor H polypeptide). In another embodiment, a separation-based assay (e.g., PAGE) is used to diagnose AMD or other diseases in an individual by detecting the presence or absence of the variant Factor H polypeptide in a biological sample, e.g., a serum sample, of the individual.

A kit can be supplied for use in performing any one or more of the methods of diagnosing an individual's propensity to develop AMD. The kit can include one or more allele-specific oligonucleotides, such as allele-specific primers or probes, or antibodies that specifically recognize a macromolecule exhibiting an allelic variant of interest. The allele-specific oligonucleotides can include sequences derived from the coding (exons) or non-coding (promoter, 5′ untranslated, introns or 3′ untranslated) regions, according to the location of the particular allelic variant that is sought to be identified as present or absent. Alternatively, the probes can be antibody probes, to be used to recognize the normal or wild-type polypeptide or variant polypeptides in which one or more non-synonymous single nucleotide polymorphisms (SNPs) are present in the coding region. Kits should further include a set of instructions setting forth a protocol that includes the steps of the particular method for which the kit is intended to be used. The kits may be used to diagnose AMD based on the allelic variants and haplotypes described herein.

In another aspect is a method of treating an individual shown to have an increased risk for developing AMD for age-related macular degeneration, comprising administering to the individual a pharmaceutically effective amount of a composition, wherein said individual is characterized in that a biological sample obtained from said individual exhibits the allelic variant of any one of claims 1-6, and wherein said composition comprises an agent to cancel the association between said allelic variant and development of AMD. Compositions useful in such methods of treatment include agents, where the agent is an RNA complementary to at least a portion of the nucleotide sequence of the allelic variant. The agent can also be an RNA that has a sequence that is an antisense RNA to the allelic variant sequence. Alternatively, the composition can include an RNA agent that is a ribozyme, or a short interfering RNA (siRNA).

### BRIEF DESCRIPTION OF THE DRAWINGS

FIG. 1 is schematic diagram showing the degree of linkage disequilibrium (D′) between the SNPs of the CFH gene that are listed in Table 4. Each of the 25 SNPs is identified along the horizontal axis of the linkage disequilibrium plot, or “HapMap”. Each cross cell box contains the pair-wise measurement of the degree of linkage disequilibrium between any two SNPs (D′). The higher the degree of LD, the darker the square box. Linkage disequilibrium is indicated by the gradient bar above the LD plot. Abbreviations: SNP, Single Nucleotide Polymorphism; LD, Linkage disequilibrium. Heavy set lines are drawn around three haplotype blocks, where block 1 includes the SNPs above horizontal positions 1 and 2, block 2 includes the includes the SNPs above horizontal positions 5-18, and block 3 includes the SNPs above the horizontal positions 19-24.

FIG. 2 is an LD (r²) plot between the 25 SNPs of the CFH gene that are listed in Table 4. Linkage disequilibrium is indicated by the gradient bar above the LD plot. The linkage disequilibrium between any two SNPs is listed in the cross cell. The darker the square, the higher the linkage disequilibrium between any two SNPs. Abbreviations: SNP, Single Nucleotide Polymorphism; LD, Linkage disequilibrium.

FIG. 3 is a table showing data derived from FBAT and conditional logistic regression analysis under an additive model for the two haplotypes identified in haplotype block 1 of the LD plot of CFH-related SNPs shown in FIGS. 1 and 2. Estimated haplotypes with allele frequency greater than 0.05 were listed and tested for association. When considering all possible haplotypes together, the resulting p value from 100,000 permutations was 0.000530.

FIG. 4 is a table showing data derived from FBAT and conditional logistic regression analysis under an additive model for the three haplotypes identified in haplotype block 2 of the LD plot of CFH-related SNPs shown in FIGS. 1 and 2. Estimated haplotypes with allele frequency greater than 0.05 were listed and tested for association. When considering all possible haplotypes together, the resulting p value from 100,000 permutations was 0.000010.

FIG. 5 is a table showing data derived from FBAT and conditional logistic regression analysis under an additive model for the four haplotypes identified in haplotype block 3 of the LD plot of CFH-related SNPs shown in FIGS. 1 and 2. Estimated haplotypes with allele frequency greater than 0.05 were listed and tested for association. When considering all possible haplotypes together, the resulting p value from 100,000 permutations was 0.005280. “n/a”=non-applicable.

FIG. 6 is an LD (D′) plot between the 28 SNPs of the C3 gene that are listed in Table 4. Associations between the 28 SNPs result in six distinct haplotype blocks. Linkage disequilibrium is indicated by the gradient bar above the LD plot. The linkage disequilibrium between any two SNPs is listed in the cross cell. The darker the square, the higher the linkage disequilibrium between any two SNPs. Abbreviations: SNP, Single Nucleotide Polymorphism; LD, Linkage disequilibrium.

FIG. 7 is an LD (r²) plot between the 28 SNPs of the C3 gene that are listed in Table 4. Associations between the 28 SNPs result in six distinct haplotype blocks. Linkage disequilibrium is indicated by the gradient bar above the LD plot. The linkage disequilibrium between any two SNPs is listed in the cross cell. The darker the square, the higher the linkage disequilibrium between any two SNPs. Abbreviations: SNP, Single Nucleotide Polymorphism; LD, Linkage disequilibrium.

FIG. 8 is a table showing data derived from FBAT and conditional logistic regression analysis under an additive model for the two haplotypes identified in haplotype block 3 of the LD plot of C3-related SNPs shown in FIGS. 6 and 7. Estimated haplotypes with allele frequency greater than 0.05 were listed and tested for association. When considering all possible haplotypes together, the resulting p value from 5118 permutations was 0.127100. “n/a”=non-applicable.

FIG. 9 is a table showing data derived from FBAT and conditional logistic regression analysis under a recessive model for the two haplotypes identified in haplotype block 5 of the LD plot of C3-related SNPs shown in FIGS. 6 and 7. Estimated haplotypes with allele frequency greater than 0.05 were listed and tested for association. When considering all possible haplotypes together, the resulting p value from 9497 permutations was 0.050753. “n/a”=nonapplicable.

FIG. 10 is schematic diagram showing the degree of linkage disequilibrium (D′) between the eight SNPs of the CFD gene that are listed in Table 4. Each of the eight SNPs is identified along the horizontal axis of the linkage disequilibrium plot, or “HapMap”. Each cross cell box contains the pair-wise measurement of the degree of linkage disequilibrium between any two SNPs (D′). The higher the degree of LD, the darker the square box. Linkage disequilibrium is indicated by the gradient bar above the LD plot. Abbreviations: SNP, Single Nucleotide Polymorphism; LD, Linkage disequilibrium. Heavy set lines are drawn around two haplotype blocks, where block 1 includes the SNPs above horizontal positions 5 and 6, and block 2 includes the SNPs above horizontal positions 7 and 8.

FIG. 11 is an LD (r²) plot between the eight SNPs of the CFD gene that are listed in Table 4. Linkage disequilibrium is indicated by the gradient bar above the LD plot. The linkage disequilibrium between any two SNPs is listed in the cross cell. The darker the square, the higher the linkage disequilibrium between any two SNPs. Abbreviations: SNP, Single Nucleotide Polymorphism; LD, Linkage disequilibrium.

### DETAILED DESCRIPTION

Using a retrospective matched-pair case-control study of a hospital clinic-based sample of Extremely Discordant Sibpairs (“EDSP”), Applicant has examined how ten genes of the complement factor pathway influence risk for neovascular age-related macular degeneration (AMD): the ten genes being Complement Factor H(CFH), Complement Factor D (CFD), Complement Component 2 (C2), Complement Component 3 (C3), Complement Component 1, Subcomponent Q, A chain (C1QA), Complement Component 1, Subcomponent Q, B chain (C1QB), Complement Component 1, Subcomponent Q, C chain (C1QC), Complement Factor I (CFI), Complement Factor B (CFB), and Complement Component 5A, Receptor 1 (C5AR1). The EDSP cohort consisted of 134 unrelated patients with neovascular age-related macular degeneration (AMD) who have a sibling with normal maculae (collectively, 268 subjects).

Each of the ten genes listed in the paragraph above was analyzed by performing a combination of the Sequenom iPLEX system technology (Sequenom®, San Diego Calif.) and direct sequencing on samples of leukocyte DNA obtained from each of the 268 subjects. The entire coding region of each gene was sequenced, as well as one megabase into the promoter region, and including the acceptor/donor splice sites, for each of the ten genes. Based on this sequencing, 103 SNP variants were identified as being represented in the EDSP cohort for the ten genes sequenced in each subject. A list of the SNP variants identified within the EDSP cohort is shown in Tables 3 and 4.

Seventy of the 103 SNP variants were present in the EDSP cohort at more than five percent minor allele frequency (MAF >5%) (Table 4). The statistical technique of single SNP conditional logistic regression (CLR) was applied to each of the seventy SNPs that were seen in the EDSP cohort population at more than five percent minor allele frequency (MAF >5%). Based on the results of single CLR, significant association (p<0.05 with AMD was seen only in SNPs within the CFH, CFD and C3 genes. Thus, further statistical analysis was performed for the SNPs within these three genes alone.

Further analysis of significant association between the CFH, CFD, and C3 SNP variant and AMD was conducted using the family based association test (FBAT). FBAT analysis without Bonferroni correction indicated a significant association between wet AMD and sixteen SNPs (one in CFD, three in C3 and twelve in CFH), as shown in Tables 6(a-c). After applying a Bonferroni correction to the FBAT data, only the twelve CFH-related SNP variants (rs800292, rs35507625, rs572515, rs1061147, rs7529589, rs482934, rs1061170, rs12038333, rs2274700, rs203674, rs375046 and rs16840522) continued to exhibit a significant association with AMD. Of these SNP variants, rs572515 was the most significant after applying a Bonferroni correction (p=6.2E-06), and was associated with more than a 71-fold increase in the risk of AMD. Two of these SNPs, rs482934 and rs375046, are shown to have an association with an increased risk for neovascular AMD (p=2.10E-05, and p=9.84E-04, respectively) for the first time.

Multiple conditional logistic regression analysis was then performed on the twelve CFH-related SNPs identified in the paragraph above as having survived Bonferroni correction. CFH-related SNP variants rs572515, rs482934, rs2274700, and rs203674 were again identified as having the most significant association with AMD risk. Results from the multiple conditional logistic regression analysis further indicated that SNP rs572515 continued to have the most significant association with neovascular AMD, after controlling for known risk factors.

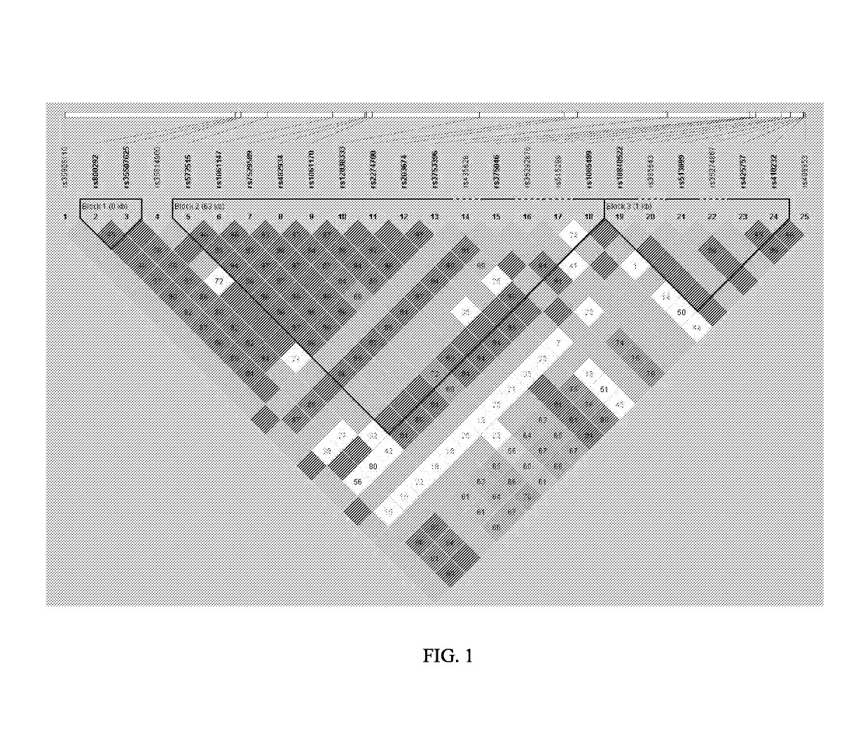

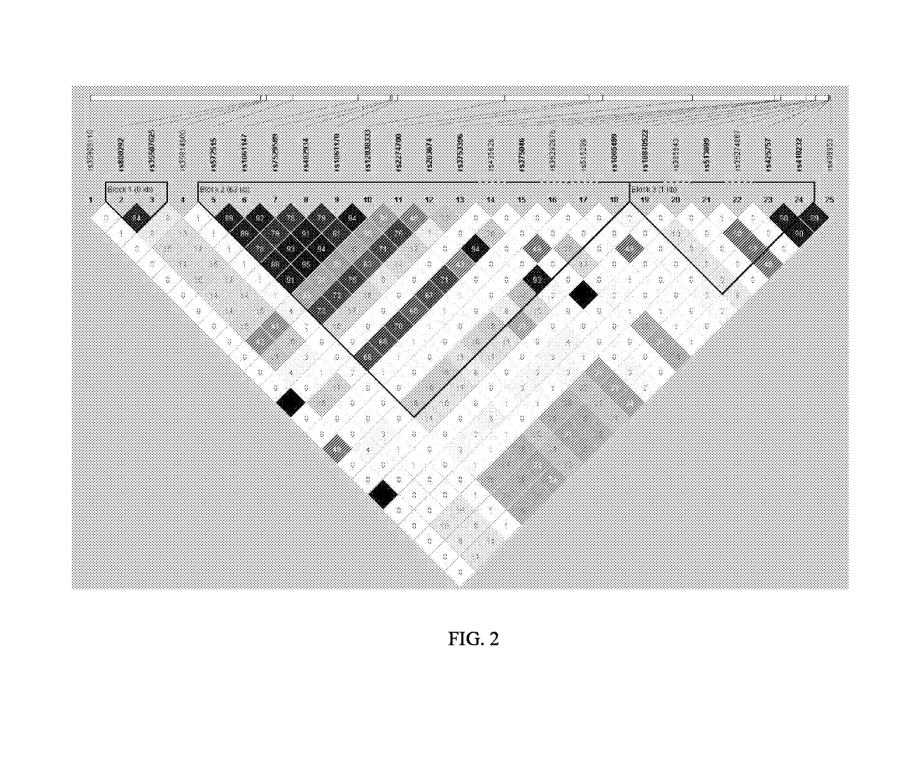

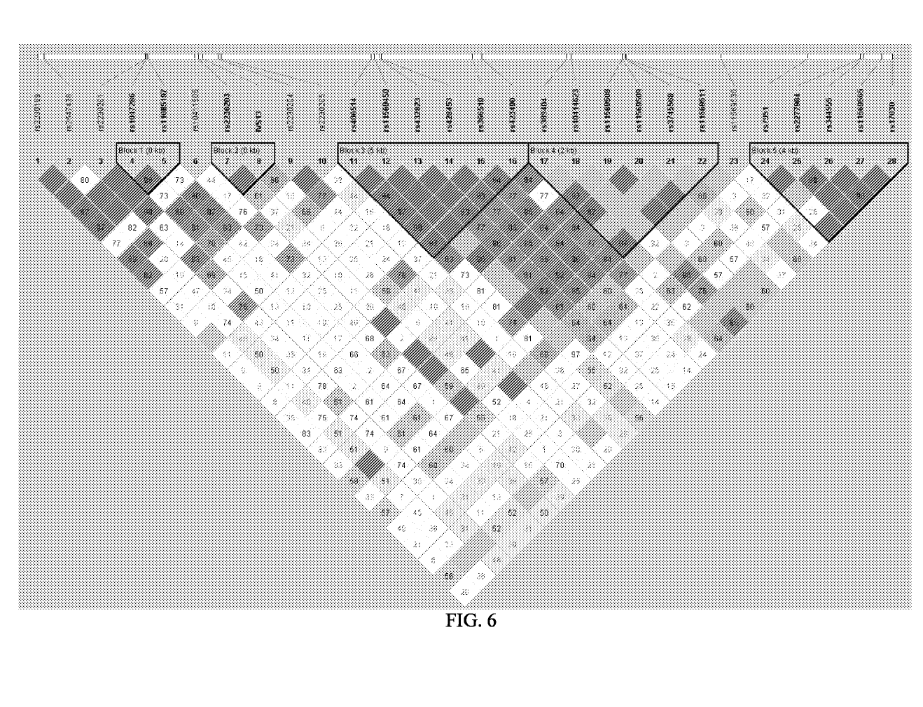

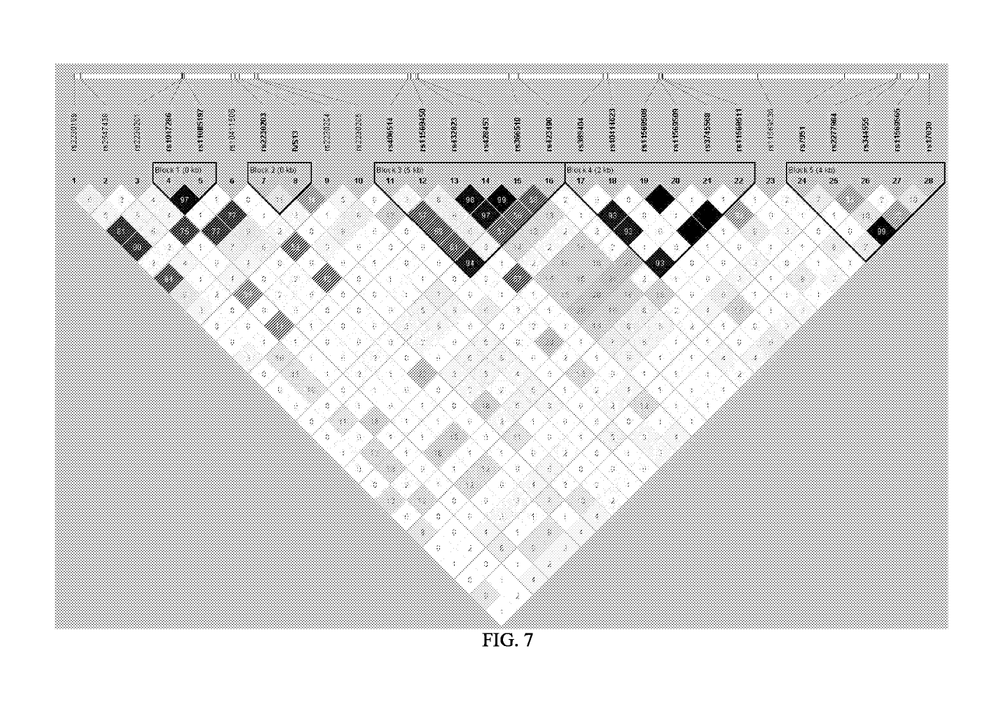

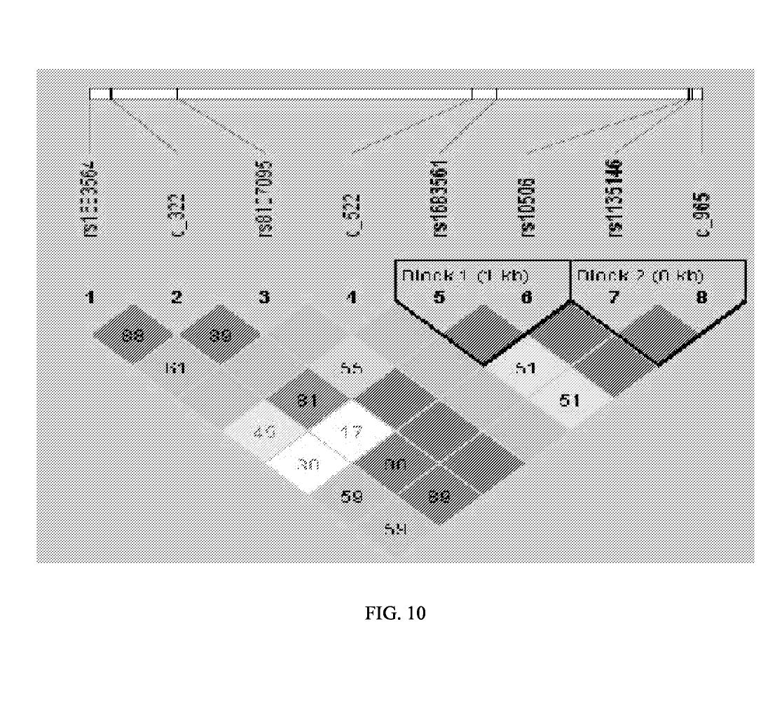

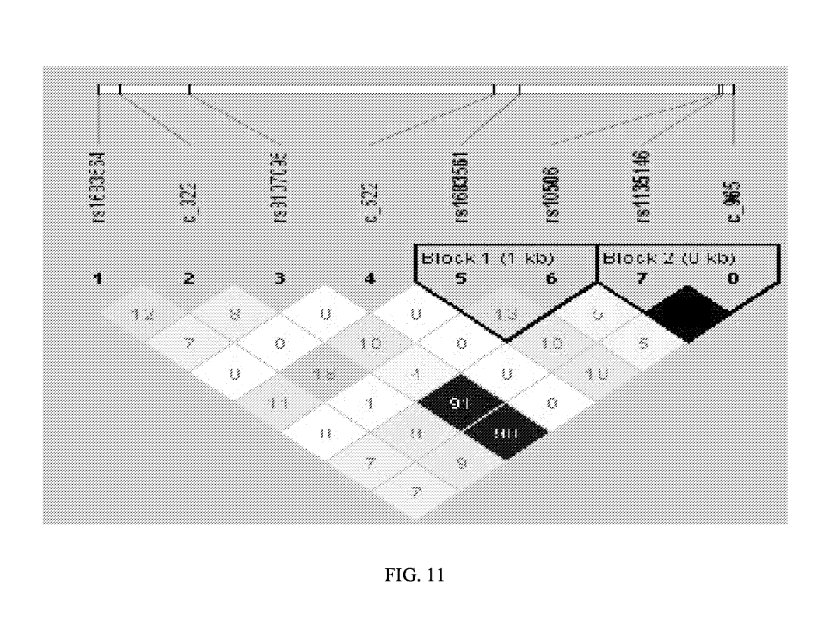

Linkage disequilibrium plots (r² and D) were generated for each of the three genes CFH (FIGS. 1 and 2), C3 (FIGS. 6 and 7), and CFD (FIGS. 10 and 11), respectively. Linkage disequilibrium analysis between the 25 genotyped SNPs in the CFH gene listed in Table 4 revealed three distinct haplotype blocks, which were defined by confidence intervals using the Haploview algorithm proposed by Gabriel et al., supra. The same analysis produced five haplotype blocks within the 28 genotyped SNPs of C3 listed in Table 4, and two haplotype blocks within the eight genotyped SNPs of CFD listed in Table 4.

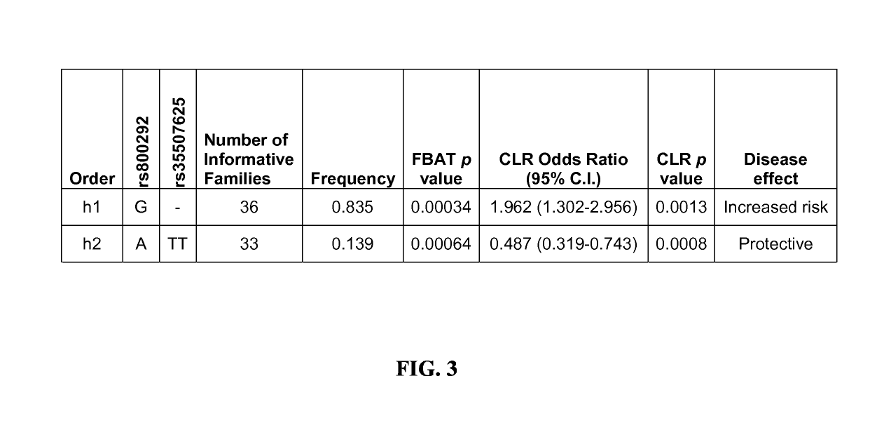

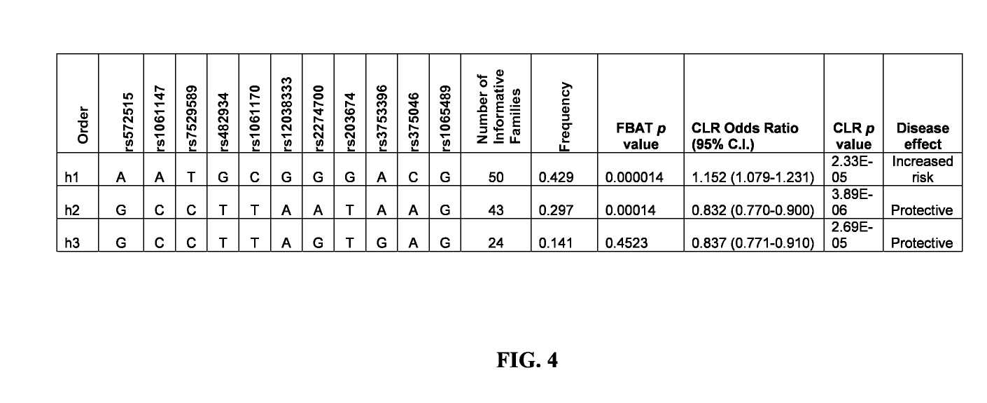

The haplotypes were tested with FBAT to determine the best fit for each genotypic model tested (additive, dominant, or recessive). Haplotype analysis using FBAT demonstrated that both CFH and C3 contained significant haplotypes, nine novel haplotypes across the three CFH haplotype blocks (FIGS. 3, 4, and 5), of which six show statistically significant association with AMD. Of these six CFH haplotypes, the most significant was a protective haplotype within CFH Haplotype Block 2, under an additive model (FBAT p=0.00014)(FIG. 4).

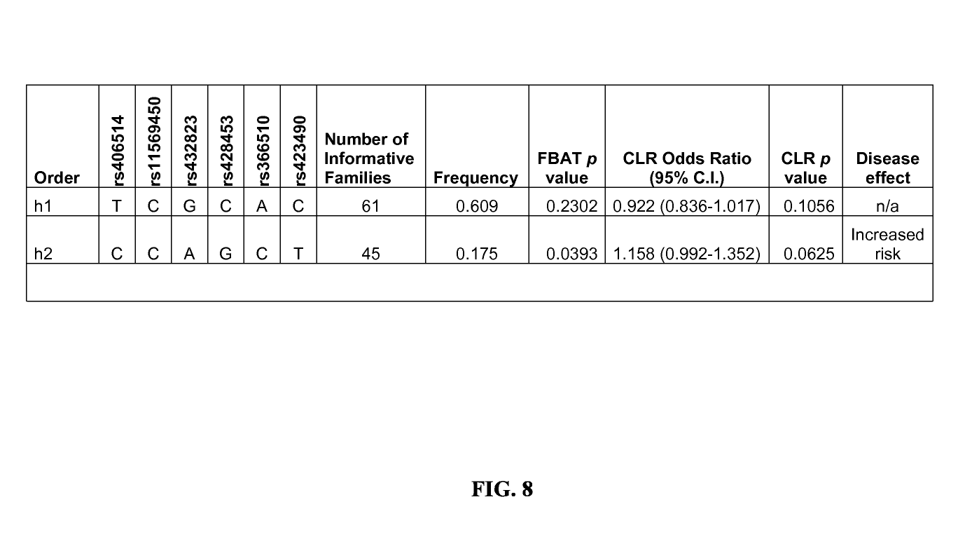

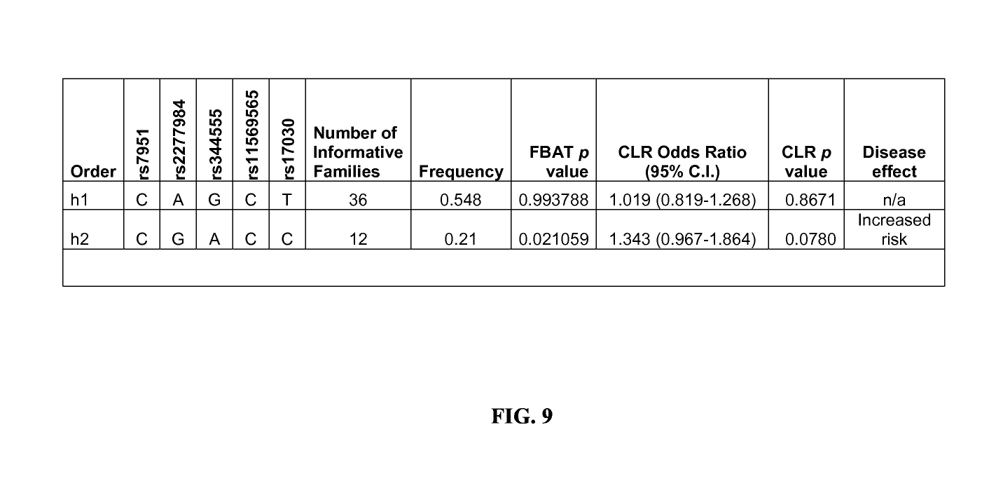

Haplotype analysis of the C3 gene revealed four novel C3 haplotypes across two of the C3 haplotype blocks (FIGS. 8 and 9). One of these novel C3 haplotypes, a risk associated C3 haplotype in C3 haplotype Block 5 (h2, FIG. 9), was statistically significant by FBAT for association with AMD under a recessive model (p=0.021059).

The haplotypes were further tested by conditional logistic regression analysis. Only the CFH haplotypes were significant according to CLR. Haplotype analysis of the CFH gene revealed six novel significant haplotypes across the three haplotype blocks (FIGS. 3, 4, and 5). Of these six haplotypes, the most significant was a protective haplotype within CFH Haplotype Block 2, under an additive model (CLR p=3.89E-6))(FIG. 4). None of the C3 haplotypes were significant according to conditional logistic regression analysis.

### DEFINITIONS

The following definitions are provided to aid in understanding the invention. Unless otherwise defined, all terms of art, notations and other scientific or medical terms or terminology used herein are intended to have the meanings commonly understood by those of skill in the arts of medicine and molecular biology. In some cases, terms with commonly understood meanings are defined herein for clarity and/or for ready reference, and the inclusion of such definitions herein should not be assumed to represent a substantial difference over what is generally understood in the art.

A “nucleic acid”, “polynucleotide” or “oligonucleotide” is a polymeric form of nucleotides of any length, may be DNA or RNA, and may be single- or double-stranded. Nucleic acids may include promoters or other regulatory sequences. Oligonucleotides are usually prepared by synthetic means. Unless otherwise stated, nucleic acids include segments of DNA or RNA or their respective complements spanning or flanking any one of the sequences set forth herein The segments are usually between 5 and 100 contiguous bases, and often range from a lower limit of 5, 10, 12, 15, 20, or 25 nucleotides to an upper limit of 10, 15, 20, 25, 30, 50 or 100 nucleotides (where the upper limit is greater than the lower limit). Nucleic acids between 5-10, 5-20, 10-20, 12-30, 15-30, 10-50, 20-50 or 20-100 bases are common. The polymorphic site can occur within any position of the segment. A reference to the sequence of one strand of a double-stranded nucleic acid defines the complementary sequence and except where otherwise clear from context, a reference to one strand of a nucleic acid also refers to its complement. For certain applications, nucleic acid molecules may be modified to increase intracellular stability and half-life. Possible modifications include, but are not limited to, the use of phosphorothioate or 2′-O-methyl rather than phosphodiesterase linkages within the backbone of the molecule. Modified nucleic acids include peptide nucleic acids (PNAs) and nucleic acids with nontraditional bases such as inosine, queosine and wybutosine and acetyl-, methyl-, thio- and similarly modified forms of adenine, cytosine, guanine, thymine, and uridine which are not as easily recognized by endogenous endonucleases. An “isolated” nucleic acid means a nucleic acid species that is the predominant species present in a composition. “Isolated” means that the nucleic acid is separated from at least one compound with which it is associated in nature. A purified nucleic acid comprises (on a molar basis) at least about 50, 80 or 90 percent of all macromolecular species present.

“Hybridization probes” are nucleic acids capable of binding in a base-specific manner to a complementary strand of nucleic acid. Such probes include nucleic acids and peptide nucleic acids (Nielsen et al., Science, 254:1497-1500, 1991). Hybridization may be performed under stringent conditions which are known in the art. For example, see, e.g., Berger and Kimmel (1987) Methods In Enzymology, Vol. 152: Guide To Molecular Cloning Techniques, San Diego: Academic Press, Inc.; Sambrook et al. (1989) Molecular Cloning: A Laboratory Manual, 2nd Ed., Vols. 1-3, Cold Spring Harbor Laboratory; Sambrook (2001) 3rd Edition; Rychlik, W. and Rhoads, R. E., 1989, Nucl. Acids Res. 17, 8543; Mueller, P. R. et al. (1993) In: Current Protocols in Molecular Biology 15.5, Greene Publishing Associates, Inc. and John Wiley and Sons, New York; and Anderson and Young, Quantitative Filter Hybridization in Nucleic Acid Hybridization (1985)). As used herein, the term “probe” includes primers. Probes and primers are sometimes referred to as “oligonucleotides.”

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer but typically ranges from 15 to 30 nucleotides. A primer sequence need not be exactly complementary to a template but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

Exemplary hybridization conditions for short probes and primers is about 5 to 12 degrees C. below the calculated Tm. Formulas for calculating Tm are known and include: Tm=4° C.×(number of G's and C's in the primer)+2° C.×(number of A's and T's in the primer) for oligos<14 bases and assumes a reaction is carried out in the presence of 50 mM monovalent cations. For longer oligonucleotides, the following formula can be used: Tm=64.9° C.+41° C.×(number of G's and C's in the primer—16.4)/N, where N is the length of the primer. Another commonly used formula takes into account the salt concentration of the reaction (Rychlik, supra, Sambrook, supra, Mueller, supra.): Tm=81.5° C.+16.6° C.×(log 10[Na+]+[K+])+0.41° C.×(% GC)−675/N, where N is the number of nucleotides in the oligonucleotide. The aforementioned formulae provide a starting point for certain applications; however, the design of particular probes and primers may take into account additional or different factors. Methods for design of probes and primers for use in the methods of the invention are well known in the art.

Two amino acid sequences are considered to have “substantial identity” when they are at least about 80% identical, preferably at least about 90% identical, more preferably at least about 95%, at least about 98% identical or at least about 99% identical. Percentage sequence identity is typically calculated by determining the optimal alignment between two sequences and comparing the two sequences. Optimal alignment of sequences may be conducted by inspection, or using the local homology algorithm of Smith and Waterman, 1981, Adv. Appl. Math. 2: 482, using the homology alignment algorithm of Needleman and Wunsch, 1970, J. Mol. Biol. 48: 443, using the search for similarity method of Pearson and Lipman, 1988, Proc. Natl. Acad. Sci. U.S.A. 85: 2444, by computerized implementations of these algorithms (e.g., in the Wisconsin Genetics Software Package, Genetics Computer Group, 575 Science Dr., Madison, Wis.) using default parameters for amino acid comparisons (e.g., for gap-scoring, etc.). It is sometimes desirable to describe sequence identity between two sequences in reference to a particular length or region (e.g., two sequences may be described as having at least 95% identity over a length of at least 500 basepairs). Usually the length will be at least about 50, 100, 200, 300, 400, 500, 600, 700, 800, 900 or 1000 amino acids, or the full length of the reference protein. Two amino acid sequences can also be considered to have substantial identity if they differ by 1, 2, or 3 residues, or by from 2-20 residues, 2-10 residues, 3-20 residues, or 3-10 residues.

The term “wild-type” refers to a nucleic acid or polypeptide in which the sequence is a form that is prevalent in a population.

The term “polymorphism” refers to the occurrence of two or more genetically determined alternative sequences or alleles at a particular genomic site in a population. A “polymorphic site” is a locus at which sequence divergence occurs. Polymorphic sites have at least two alleles. A “diallelic polymorphism” has two alleles. A “triallelic polymorphism” has three alleles. Diploid organisms may be homozygous or heterozygous for allelic forms. A polymorphic site may be as small as one base pair. Examples of polymorphic sites include: restriction fragment length polymorphisms (RFLPs); variable number of tandem repeats (VNTRs); hypervariable regions; minisatellites; dinucleotide repeats; trinucleotide repeats; tetranucleotide repeats; and simple sequence repeats. As used herein, reference to a “polymorphism” can encompass a set of polymorphisms (e.g., a haplotype).

Generally, a “single nucleotide polymorphism (SNP)” occurs at a polymorphic site occupied by a single nucleotide, which is the site of variation between allelic sequences. The site is usually preceded by and followed by highly conserved sequences of the allele. A SNP usually arises due to substitution of one nucleotide for another at the polymorphic site. Replacement of one purine by another purine or one pyrimidine by another pyrimidine is called a transition. Replacement of a purine by a pyrimidine or vice versa is called a transversion. A synonymous SNP refers to a substitution of one nucleotide for another in the coding region that does not change the amino acid sequence of the encoded polypeptide. A non-synonymous SNP refers to a substitution of one nucleotide for another in the coding region that changes the amino acid sequence of the encoded polypeptide. A SNP may also arise from a deletion or an insertion of a nucleotide or nucleotides relative to a reference allele. In the present application, the term SNP extends to deletion or insertion of two contiguous nucleotides, for example, the thymine-thymine (TT) insertion of rs35507625.

The term “minor allele”, as used herein, refers to the allele that is least frequent in the 134 unaffected siblings of the EDSP cohort when compared with alternative allelic variants at the same genomic position. Minor Allele Frequency (MAF) (unaffected)” refers to the frequency of the minor allele in the 134 unaffected siblings of the EDSP cohort, and “Frequency in Affected” refers to the frequency of that same allele in the 134 affected index patients. Thus, the MAF refers to the frequency of the minor allele in the unaffected portion of the EDSP cohort population regardless of the frequency of that allele in the affected portion of the EDSP cohort population. Where there are only two known alleles at a particular genomic position, the alternative, non-minor, allelic variant in the unaffected population is variously referred to herein as the “major”, “ancestral”, “common”, or “referent” allele.

The terms “risk,” “protective,” and “neutral” are used to characterize the phenotypic impact of allelic variations, e.g., SNPs, haplotypes, and diplotypes. A “risk allele”, “risk SNP, or “risk haplotype” is an allelic form of a genetic locus that includes at least one variant polymorphism, or two or more variant alleles, associated with an increased risk for developing AMD. The term “variant” refers to a nucleotide sequence in which the sequence differs from the sequence most prevalent in a defined population. A variant polymorphism can be in the coding or non-coding portions of the gene. A “protective allele, “protective SNP”, or “protective haplotype” is an allelic form of a genetic locus that includes at least one variant polymorphism, and preferably a set of variant polymorphisms, associated with decreased risk of developing AMD. A “neutral allele”, “neutral SNP”, or “neutral haplotype” is an allelic form of a genetic locus that contains a variant polymorphism that is not associated in a defined population or ethnic group with either an increased or a decreased risk of developing AMD. It will be clear from the following discussion that a protein encoded in a “neutral” haplotype may be protective when administered to a patient in need of treatment or prophylaxis for AMD or other conditions. That is, both “neutral” and “protective” forms of CFH can provide therapeutic benefit when administered to, for example, a subject with AMD or risk for developing AMD, and thus can “protect” the subject by reducing its propensity for disease.

The term “haplotype” refers to a particular combination of alleles that are located at or proximal to a genetic locus or cluster of loci on a segment of a chromosome. As used herein, a “haplotype block” refers to a chromosomal segment having a level of recombination events that is lower than expected by chance, and than the frequency of recombination events occurring at the boundaries of such blocks.

“Linkage disequilibrium” is a measure of the degree of non-random association between alleles, genetic markers, and/or polymorphisms.

### Extremely Discordant Sibpair Population (“EDSP Cohort”)

The results reported herein were obtained by studying a phenotypically well-defined cohort of 268 subjects that included 134 extremely discordant sibpairs. This population, referred to herein as an Extremely Discordant Sibpair Population, or “EDSP cohort”, was assembled using Extremely Discordant Sibpair recruitment techniques. “Extremely Discordant Sibpair”, as used herein, refers to a pair of siblings in which one sibling exhibits in the upper 10% of disease severity (referred to herein as an “affected sibling”, as an “index patient” or as a “proband”) and the other sibling (“unaffected sibling” or “control subject”) exhibits in the bottom 10% to 30% of disease severity.

The protocol for recruitment of the EDSP cohort set forth below was reviewed and approved by the Institutional Review Boards at the Massachusetts Eye & Ear Infirmary (MEEI), Boston, Mass., and William Beaumont Hospital, Royal Oak, Mich., and conforms to the tenets of the Declaration of Helsinki Eligible patients were enrolled in this study after they gave informed consent either in person, over the phone, or through the mail, before answering questions to a standardized questionnaire and donating 10 to 50 ml of venous blood.

Index patients with neovascular AMD were recruited from the Retina Service of the Massachusetts Eye and Ear Infirmary. By the terms “index patient” and “proband” are meant the patient or member of the family that brings a family under study. Index patients and control subjects were evaluated clinically. To be included in the study, all index patients had to be aged 50 years or older. Patients exhibiting a neovascular degeneration of the macula who are younger than 50 years of age have a significant possibility of suffering from one of a number of diseases that mimic AMD.

An additional subset of the EDSP cohort was formed for association studies by including siblings with the early dry form of AMD (AREDS category 2) whom are 65 years or older. Analyzing this additional cohort helped to pinpoint factors associated with disease severity (i.e., wet versus mild or very early AMD). It has been shown that siblings in the bottom 30%-50% (AREDS category 2) of disease severity may be as powerful for uncovering disease risk alleles associated with a complex disease as those in the bottom 10% of disease severity (AREDS category 1 or less). Risch, N. et al., “Extreme discordant sib pairs for mapping quantitative trait loci in humans”, Science 268:1584-89 (1995); Risch, N. J. et al., “Mapping quantitative trait loci with extreme discordant sib pairs: sampling considerations”, Am. J. Hum. Genet., 58:836-43 (1996). An additional group of subjects was added to the EDSP cohort by including siblings over the age of 65 years who were AMD-free (AREDS category 1 or less), regardless of the proband's (i.e., index patient's) initial diagnosis of neovascular AMD.

Inclusion in the EDSP cohort population required that all index patients have the neovascular form of AMD in at least one eye. For an index patient to be considered to have “neovascular AMD” for purposes of inclusion in the EDSP cohort, it was required that sub-retinal hemorrhage, fibrosis, or fluorescein angiographic presence of neovascularization have been documented at the time of or before enrollment in the study. Patients whose only exudative finding was a retinal pigment epithelium detachment were excluded, due to the possibility that detachment of the retinal pigment epithelium alone may not represent neovascular AMD and, therefore, the severe phenotype that was sought for this patient cohort. Patients with signs of pathologic myopia, presumed ocular histoplasmosis syndrome, angioid streaks, choroidal rupture, or any hereditary diseases other than AMD, or who had had prior laser treatment due to retinal conditions other than AMD, were also excluded.

Each recruited index patient was asked if he or she had a sibling with no history of AMD that was past the age at which the index patient first had neovascular AMD diagnosed and/or was greater than 65 years of age. After obtaining informed consent from the index patient, those siblings who had a sibling with no history of AMD that was past the age at which the index patient first had neovascular AMD diagnosed were contacted, their consent to enter the study was obtained, and their ocular history was reviewed. If a sibling was apparently eligible as an unaffected sibling, that status was documented by obtaining fundus photographs according to methods familiar to those of ordinary skill in the art, or by arranging to have copies of pre-existing fundus photographs submitted for review. By “fundus” is meant the interior surface of the eye, opposite the lens, including the retina, optic disc, macula and fovea, and posterior pole. The fundus can be viewed with an ophthalmoscope. By “fundus photograph” is meant a photograph taken of the pupil during a dilated fundus examination, a diagnostic procedure that employs the use of mydriatic eye drops to dilate or enlarge the pupil in order to obtain a better view of the fundus with an ophthalmoscope. DFE has been found to be a more effective method for evaluation of internal ocular health than non-dilated examination. A few unaffected siblings were unwilling to come to an ophthalmologist's office for fundus photographs, or they were too frail to do so, but they were amenable to a home visit by one of the investigators for a dilated fundus examination.

Tests were conducted to validate that sibpairs are likely full siblings, and to determine that there was no substantial contamination of the EDSP cohort with sibpairs who were unknowingly half siblings or who were unrelated. To do so, blood samples were collected from both index and unaffected siblings. DNA was purified from each of these blood samples and then analyzed with three highly polymorphic microsatellite markers (D2S428, D5S816, D7S796) with a heterozygosity of 94% by methods known to those skilled in the art. See, Nussbaum et al., Thompson and Thompson Genetics in Medicine, (7^{th}. ed., Saunders Elsevier, 2007). DNA from a subset of 48 of the sib pairs was analyzed, with three unlinked microsatellite markers (D2S428, D5S816, D7S796) each having a heterozygosity of 94%. At each marker locus, records were made of the number of alleles (0, 1, or 2) shared by each pair of siblings, which is defined as the identity-by-state score. Summing all three markers and excluding occasional samples that did not work for one or two markers, 25 instances of an identity-by-state score of 0, 67 of an identity-by-state score of 1, and 41 of an identity-by-state score of 2 was found. This finding was compared with the expected identity by-state score totals for markers with a heterozygosity of 94% (30, 66, and 37, respectively). There was no statistically significant difference between the observed and expected distributions of identity-by-state scores (χ^{2/3}=0.67, P>0.75). This result suggests that there is no major contamination of the recruited discordant sib pairs with half siblings or unrelated pairs.

The unaffected siblings had normal maculae at an age older than that at which the index patient was first diagnosed with neovascular AMD. Unaffected siblings had normal maculae (defined as the zone centered at the foveola and extending two disc diameters, or 3000 microns, in radius), and fulfilled the following criteria: 0 to 5 small drusen (all less than 63 micrometers in diameter), no pigment abnormalities, no geographic atrophy, and no neovascularization (AMD “category 1” on the AREDS scale). Criteria for classifying neovascularization is familiar to those of ordinary skill in the art; see, e.g., Macular Photocoagulation Study Group, “Risk factors for choroidal neovascularization in the second eye of patients with juxtafoveal or subfoveal choroidal neovascularization secondary to age-related macular degeneration”, Arch Ophthalmol., 115:741-747, 1997; Age-Related Eye Disease Study Research Group, “The Age-Related Eye Disease Study system for classifying age-related macular degeneration from stereoscopic color fundus photographs: the Age-Related Eye Disease Study report number 6”, Am J Ophthalmol, 132:668-81, 2001. The disease status of each participant was confirmed by at least two investigators, by means of fundus photography or fluorescein angiography, except in four cases (n=4) in which one of the investigators conducted a home examination.

Certain additional characteristics were defined for each subject in the EDSP cohort, including those characteristics previously determined to be risk factors for AMD, such as age and smoking. All participants were Caucasian, however every effort was made to recruit and include other racial and ethnic groups. The low numbers of non-Caucasians in the EDSP cohort may be due to the low prevalence of neovascular AMD in populations other than Caucasians such as Hispanics and Blacks. Klein R, et al., “Prevalence of age-related macular degeneration in 4 racial/ethnic groups in the multi-ethnic study of atherosclerosis”, Ophthalmology, 2006; 113:373-380.

Table 1 sets forth the age characteristics of the extremely discordant sibpair population evaluated herein, where “age” refers to the age of the index patient and separately for the age of the unaffected sibling at the time of enrollment into the study and thus the age at which fundus photographs were examined and samples of leukocyte DNA were collected.

To collect data and to ascertain exposures to other variables known to impact AMD, interviews of participants were conducted in person or by telephone. After a sibpair was deemed eligible, a standardized questionnaire was administered to all participants to ascertain potential risk factors for AMD such as smoking exposure. The age of the index patient at the time of the fundus photographs was used as the cutoff reference age for smoking exposure for both members in a sibship. In most cases, the diagnosis of AMD was made simultaneously with the diagnosis of neovascular AMD. If a participant had ever smoked, records were kept of the age when they started smoking, the age when they quit smoking (if they did quit), and the average number of packs of cigarettes smoked per day. Based on the responses, the number of pack-years of cigarettes smoked was calculated for each smoker. Participants who smoked less than 100 cigarettes during their lifetime (i.e., less than 1/73 of a pack-year) were categorized as having never smoked. A pack-year was defined as one pack of cigarettes per day for one year, with one pack defined as twenty cigarettes. Pack-years of smoking has been previously identified as a significant predictor of neovascular AMD. Specifically, each pack year of smoking has been associated with a 2% increase in the risk of neovascular AMD (Odds Ratio (“OR”), 1.02; 95% CI, 1.01-1.04; p=0.007). Kim et al, “Comprehensive analysis of CRP, CFH Y402H and environmental risk factors on risk of neovascular age-related macular degeneration”, Mol. Vision, 14:1487-1495 (2008); DeAngelis MM, et al., “Cigarette Smoking, CFH, APOE, ELOVL4, and Risk of Neovascular Age-Related Macular Degeneration”, Arch Ophthalmol, 125:49-54 (2007); DeAngelis M M, et al., “Extremely discordant sib-pair study design to determine risk factors for neovascular age-related macular degeneration”, Arch Ophthalmol. 122:575-580 (2004).

Replication of data was done using three case control cohorts from the Casey Eye Institute (CEI) (Portland, Oreg.). In particular any previously unreported genes shown to have association with AMD in the MEEI cohort were then tested in three separate AMD cohorts from the Casey Eye Institute (CEI), Portland, Oreg.: (1) a cohort of extended families genetically enriched for their predisposition to the disease (1253 Caucasian affected and unaffected individuals from 124 extended families (average age 68.2 years; 492 males, 761 females); (2) a sporadic case/control advanced AMD cohort (211 sporadic cases (age 79 years, range 57-100; 70 male, 141 female; 210 Caucasian, 1 other racial origin); and 183 unrelated ophthalmologically evaluated controls (age 74, range 63-92; 81 male, 102 female; 183 Caucasian); and (3) the Age-Related Eye Disease Study (AREDS) cohort (2201 cases and controls). Further details of studies conducted on these cohorts are publicly available; see, Yang et al., N. Engl. J. Med., 359(14):1456-63, 2008; Francis et al., Mol. Vis., 14:1395-1400, 2008; Francis et al., PLoS ONE, 2 (11):e1197, 2007.

In Table 1, the reference age for all unaffected siblings of the index patient was the reference age of the index patient. In sibships in which more than one sibling was affected (n=4), the affected sibling with the earliest age of diagnosis was used as the reference age for all siblings. As used herein, the term “sibship” refers to the group or number of children produced by a pair of parents; thus, a set of siblings constitute a sibship.

The following factors were deemed to be insignificant for AMD, and thus were not considered: sex, iris color, education, alcohol consumption, body mass index (BMI), multivitamin use, vitamin C use, vitamin E use, hypercholesterolemia, aspirin use, hypertension, other cardiovascular risk factors (angina, bypass surgery, myocardial infarction, stroke, or transient ischemic attack), autoimmune disease (thyroid disease, psoriasis or eczema, systemic lupus, multiple sclerosis, rheumatoid arthritis), and non-insulin-dependent diabetes mellitus. See, Kim et al, “Comprehensive analysis of CRP, CFH Y402H and environmental risk factors on risk of neovascular age-related macular degeneration”, Mol Vision, 14:1487-95 (2008); DeAngelis, M. M., et al., “Extremely discordant sib-pair study design to determine risk factors for neovascular age-related macular degeneration”, Arch Ophthalmol. 122:575-80 (2004).

### Genotyping Analysis

A sample of leukocyte DNA was obtained from each of the 268 subjects within the EDSP cohort. Genotype analysis of the leukocyte DNA of each subject was conducted by Sequenom iPLEX system technology and by direct sequencing protocols.

Leukocyte DNA was purified by using either standard phenol-chloroform extraction protocols, the DNAzol® nucleic acid extraction reagent (Invitrogen Corporation, Carlsbad, Calif.) or a modified proteinase K-phenol/chloroform extraction method for purifying DNA (Qiagen, Inc., Valencia, Calif.). Additional methods of purifying leukocyte DNA are known to those skilled in the art. DNA suitable for direct sequence analysis can also be obtained from other body fluids and tissues types as known to those skilled in the art, such as without limitation saliva, blood and various types of cell lines.

### Genetic Sequence Information Relating to the CFH Gene and Variants Thereof.

The nucleic acid sequence of the Complement Factor H(CFH) gene and associated transcripts, exons, and polypeptides were obtained from ENSEMBL. The CFH gene is also referred to by the synonyms ARMS1, FHL1, HF, HF1, HF2, and is assigned the ENSEMBL gene identification number ENSG00000000971. The CFH is found on chromosome 1, at ENSEMBL location 194,887,764-194,983,255, and the start of the gene is located at ENSEMBL Contig AL049744.8.1.150626. The complete nucleic acid sequence of the CFH gene (ENSG00000000971) is available to the public at http://www.ensembl.org/Homo_sapiens/geneseqview?db=core;gene=ENSG00000000971 (last visited Nov. 4, 2008, hereby incorporated by reference). A map of the polymorphisms associated with the CFH gene (ENSG00000000971) is available to the public at http://www.ensembl.org/Homo_sapiens/genesnpview?db=core;gene=ENSG00000000971 (last visited Nov. 4, 2008, hereby incorporated by reference). The CFH gene produces four transcripts: (1) transcript CFH-002, assigned ENSEMBL transcript identification number ENST00000359637; (2) transcript CFH-202, assigned ENSEMBL transcript identification number ENST00000367428; (3) transcript CFH-001, assigned ENSEMBL transcript identification number ENST00000367429; and (4) transcript CFH-201, assigned ENSEMBL transcript identification number ENST00000391986. CFH-001 (ENST00000367429) has 22 exons, a transcript length of 3,992 base pairs (bp), 22 exons, and a translation length of 1231 amino acid residues. A complete map of the CFH-001 (ENST00000367429) transcript, including nucleic acid sequences, corresponding amino acid sequences, and polymorphic variations, is available to the public at http://www.ensembl.org/Homo_sapiens/transview?db=core;transcript=ENST00000367429 (last visited Nov. 4, 2008, hereby incorporated by reference).

### Genetic Sequence Information Relating to the C3 Gene and Variants Thereof.

The nucleic acid sequence of the Complement Component 3 (C3) gene and associated transcripts, exons, and polypeptides were obtained from ENSEMBL. The C3 gene is also referred to by the synonym CPAMD1, and is assigned the ENSEMBL gene identification number ENSG00000125730. The C3 gene is found on chromosome 19, at ENSEMBL location 6,628,846, and the start of the gene is located at ENSEMBL ContigAC008760.7.1.200167. The complete nucleic acid sequence of the C3 gene (ENSG00000125730) is available to the public at to http://www.ensembl.org/Homo_sapiens/geneseqview?db=core;gene=ENSG00000125730 (last visited Nov. 7, 2008, hereby incorporated by reference). A map of the genomic sequence variation in the C3 gene (ENSG00000125730) is available to the public at http://www.ensembl.org/Homo_sapiens/genesnpview?db=core;gene=ENSG00000125730 (last visited Nov. 7, 2008, hereby incorporated by reference). The C3 gene (ENSG00000125730) is described by ENSEMBL as the Complement C3 precursor (C3 and PZP-like alpha-2-macroglobulin domain-containing protein 1), containing the Complement C3 beta chain, the Complement C3 alpha chain, the C3a anaphylatoxin, the Complement C3b alpha′ chain, and the Complement C3c alpha′ chain fragment 1. The C3 gene produces transcript C3-001, assigned ENSEMBL transcript identification number ENST00000245907. C3-001 (ENST00000245907) has 41 exons, a transcript length of 5,101 base pairs (bp), and a translation length of 1663 amino acid residues. A complete map of the C3-001 (ENST00000245907) transcript, including nucleic acid sequences, corresponding amino acid sequences, and polymorphic variations, is available to the public at http://www.ensembl.org/Homo_sapiens/transview?db=core;transcript=ENST00000245907 (last visited Nov. 7 2008, hereby incorporated by reference).

### Additional Genetic Sequence Information Relating to Genes and Variants of Interest

The nucleic acid sequences of additional genes considered herein and their associated transcripts, exons, and polypeptides are available to those skilled in the art through the ENSEMBL database as follows: the Complement Factor D (CFD) gene is assigned the ENSEMBL gene identification number ENSG00000197766 (hereby incorporated by reference); Complement Component 2 (C2) is assigned the ENSEMBL/Vega identification number OTTHUMG00000004932 (hereby incorporated by reference); Complement Component 1 subcomponent Q, A chain (C1QA) gene is assigned the ENSEMBL/Vega gene identification number OTTHUMG00000002893 (hereby incorporated by reference); Complement Component 1, Subcomponent Q, B chain (C1QB) gene is assigned the ENSEMBL/Vega gene identification number OTTHUMG00000002896 (hereby incorporated by reference); Complement Component 1, Subcomponent Q, C chain (C1QC) gene is assigned the ENSEMBL/Vega gene identification number OTTHUMG00000002891 (hereby incorporated by reference); Complement Factor I (CFI) gene is assigned the ENSEMBL gene identification number ENSG00000205403 (hereby incorporated by reference); Complement Factor B (CFB) gene is assigned the ENSEMBL/Vega gene identification number OTTHUMG00000004992 (hereby incorporated by reference); and the Complement Component 5A, Receptor 1 (C5AR1) gene is assigned the ENSEMBL gene to identification number ENSG00000197405 (hereby incorporated by reference).

### Primers.

Oligonucleotide primers were selected using the Primer3 program (http://frodo.wi.mit.edu/) to encompass the entire coding region and flanking intronic sequences of each gene of interest, including promoter sequence regions where transcription of RNA begins. Tables 2(a)-2(h) sets forth the nucleotide sequence of each of the primers used as follows. Table 2(a) shows the sequences of the primers used to amplify the C1QA, C1QB, and C1QC genes; Table 2b shows the sequences of the primers used to amplify the CFH Gene; Table 2c shows the sequences of the primers used to amplify the CFI Gene; Table 2d shows the sequences of the primers used to amplify the C2 Gene; Table 2e shows the sequences of the primers used to amplify the CFB Gene; Table 2f shows the sequences of the primers used to amplify the CFID Gene; Table 2g shows the sequences of the primers used to amplify the C3 Gene; Table 2h shows the sequences of the primers used to amplify the C5AR1Gene.

### Amplification.

DNA was amplified as follows. For all ten genes, the polymerase chain reaction was used to amplify genomic DNA fragments from 20 ng of leukocyte DNA from initially 92 affected patients in a solution of 10×PCR buffer containing 25 mM of MgCl₂, 0.2 mM each of dATP, dTTP, dGTP, and CTP, and 0.5 units of Taq DNA polymerase (USB Corporation, Cleveland, Ohio). For some of the amplicons of each gene 5 M Betaine was added to each PCR (Sigma-Aldrich, St. Louis, Mo.). After an initial denaturation (95° C. for 5 min), 35 cycles of PCR amplification were performed. Each cycle consisted of denaturation (95° C. for 30 sec), primer annealing (54-62° C. for 30 sec), and extension (72° C. for 30 sec). A final annealing (54-62° C. for 1.5 minutes) and extension (72° C. for 5 minutes) step completed the reaction. For preparation of sequencing reactions, PCR products were digested according to manufacturer's protocol with ExoSAP-IT (USB Corporation, Cleveland, Ohio).

### Direct DNA Sequencing:

The ExoSAP-IT/PCR products were subjected to a cycle sequencing reaction using the Big Dye Terminator v3.1 Cycle Sequencing kit (Applied Biosystems, Foster City, Calif.) according to manufacturer's protocol. Products were purified with Performa DTR Ultra 96-well plates (Edge Biosystems, Gaithersburg, Md.) in order to remove excess dye terminators. We sequenced the samples on an ABI Prism 3100 DNA sequencer (Applied Biosystems, Foster City, Calif.). Electropherograms generated from the ABI Prism 3100 were analyzed using the Lasergene DNA and protein analysis software (DNASTAR, Inc., Madison, Wis.). Two independent evaluators without knowledge of the subject's disease status read each electropherogram. All patients were sequenced in the forward direction (5′ to 3′), unless variants, polymorphisms, or mutations were identified, in which case confirmation was obtained in some cases by sequencing in the reverse direction.

Sequenom iPLEX Genotyping Analysis:

Previously identified SNPs having a minor allele frequency (MAF) of MAF >5%, and also any novel SNPs discovered within our AMD patient cohort of 92 patients, were then chosen to be analyzed via Sequenom genotyping assays among the 92 unaffected siblings to our AMD patient cohort as well as 42 additional discordant sibpairs. In total 268 samples were analyzed. Multiplex PCR assays were designed using Sequenom SpectroDESIGNER software (version 3.0.0.3) (Sequenom®, San Diego Calif.) by inputting sequence containing the SNP site and 100 bp of flanking sequence on either side of the SNP. Briefly, 10 ng genomic DNA was amplified in a 5 μl (microliter) reaction containing 1× HotStar Taq PCR buffer (Qiagen), 1.625 mM MgCl₂, 500 μM (micromolar) each dNTP, 100 nM each PCR primer, 0.5 U HotStar Taq (Qiagen). The reaction was incubated at 94° C. for 15 minutes followed by 45 cycles of 94° C. for 20 seconds, 56° C. for 30 seconds, 72° C. for 1 minute, followed by 3 minutes at 72° C. Excess dNTPs were then removed from the reaction by incubation with 0.3 U shrimp alkaline phosphatase (USB) at 37° C. for 40 minutes followed by 5 minutes at 85° C. to deactivate the enzyme. Single primer extension over the SNP was carried out in a final concentration of between 0.625 μM (micromolar) and 1.5 μM (micromolar) for each extension primer (depending on the mass of the probe), iPLEX termination mix ((Sequenom®, San Diego Calif.)) and 1.35 U iPLEX enzyme ((Sequenom®, San Diego Calif.)) and cycled using a two-step 200 short cycles program; 94° C. for 30 seconds followed by 40 cycles of 94° C. for 5 seconds, 5 cycles of 52° C. for 5 seconds, and 80° C. for 5 seconds, then 72° C. for 3 minutes. The reaction was then desalted by addition of 6 mg cation exchange resin followed by mixing and centrifugation to settle the contents of the tube. The extension product was then spotted onto a 384 well spectroCHIP before being flown in the MALDI-TOF mass spectrometer. Data was collected, real time, using SpectroTYPER Analyzer 3.3.0.15, SpectraAQUIRE 3.3.1.1 and SpectroCALLER 3.3.0.14 (Sequenom®, San Diego Calif.). Additionally, to ensure data quality genotypes for each subject were manually checked. Two of the C2 gene SNPs (rs9332739, rs547154), four of the C3 gene SNPs (rs1047286, rs11569509, rs344555, IVS13+64_{—}65delTC) and five of the CFB gene SNPs (rs4151667, rs641153, rs1048709, rs4151659, rs2072633) were not amenable to genotyping by the Sequenom iPLEX system technology and thus were directly sequenced by similar methods described previously. Because all eleven SNPs were either within or in close proximity of coding exons, oligonucleotides were designed to encompass the entire exonic region and SNP (if intronic) in one PCR product.

### Variants

Table 3 identifies the physical, chromosomal location of each of the 103 SNP variants identified in the EDSP cohort. The chromosomal location of each SNP variant was determined using the ENSEMBL program, which is available to the public by accessing the web site “ensembl.org/Homo_{—}sapiens”.

Additional characteristics of the 103 SNPs identified in the EDSP cohort are set forth in Table 4, which sets forth for each SNP: column 2, the gene that the SNP is a variant of (“gene”); column 3, the exon or intron region of that gene (“region”); column 4, the nucleotide change from major to minor allele. By way of example, G>A indicates that a guanine is the most frequent allele at that position in the unaffected population of the EDSP cohort, and that an adenine is the less frequent, or minor, allele at that position in the unaffected portion of the EDSP cohort population. In column 5, identifies the amino acid change resulting from the nucleic acid variation, if any; Column 6 identified the ENSEMBL physical location of the SNP variant. Column 7 lists the “Minor Allele Frequency (MAF) (unaffected)” which refers to the frequency of the minor allele in the 134 unaffected siblings of the EDSP cohort, while column 8 lists the “Frequency in Affected”, which refers to the frequency of the same allele in the 134 affected index patients.

Statistical Analyses on Association between SNPs and AMD

Single factor conditional logistic regression. Whether there was or was not an association between each of the 103 SNPs and AMD was tested initially using single factor conditional logistic regression, with the assistance of SAS® software (SAS® version 9.1; SAS® Institute Inc, Cary, N.C.). Results of single-factor conditional logistic regression analysis are shown in Tables 5(a-d) for the CFH gene (Table 5a), the C3 gene (Table 5b), the CFD gene (Table 5c) and for the C1QA, C1QC, C1QB, CFI, C2, CFB, and C5AR1 genes (Table 5d). Each of Tables 5(a-d) sets forth the ‘rs’ identifier of the SNP (column 1), the minor allele (column 2), the corresponding odds ratio (column 3) as determined for a particular model of inheritance (column 4), the corresponding p value of the association (column 5), and the disease effect of the association (column 6).

The genotypic model having the best fit for each genotypic model tested (i.e., additive, dominant, or recessive) is set forth in column four. One of ordinary skill in the art would understand the meaning of the terms “additive model”, “dominant model”, and “recessive model” to be consistent with the analysis set forth in Wittke-Thompson et al., “Rational Inferences about Departures from Hardy-Weinberg Equilibrium”, Am. J. Hum. Genet., 76:967-86 (2005).

The odds ratio reflects the odds of an allele carrier developing AMD, divided by the odds of a non-allele carrier developing AMD. Thus, odds ratios were calculated as the number of index patients having the minor allele times the number of unaffected subjects having the minor allele, divided by the product of the number of index patients lacking the minor allele times the number of unaffected subjects lacking the minor allele. Odds ratios were calculated at the 95% confidence interval (“C.I.”). In the context of single factor conditional logistic regression analysis, an individual SNP was judged to be associated with AMD if it had a p value of less than 0.05.

The disease effect of the association is determined based on the odds ratio and corresponding confidence interval, where an odds ratio of greater than one indicates a disease association of increased risk, an odds ratio of less than one indicates a protective association. Where an odds ratio confidence interval approximates one, the odds ratio is not considered and referred to as being not applicable (“n/a”).

As shown in Table 5a, rs482934 is associated with an increased risk of developing AMD (p=1.33E-05). The CFH-related SNP rs375046 is associated with an increased risk for AMD (p=3.21E-05). Of the CFH-related SNPs, four were identified as having the most significant association with AMD risk: rs572515, rs482934, rs2274700, and rs203674. Of these CFH-related SNPs, rs572515 was the most significant after applying a Bonferroni correction (p=6.32E-06) (Table 5a).

### Family Based Association Test (“FBAT”) Analysis of Polymorphic Variants Individually.

Those SNPs judged to be associated with AMD by virtue of having a single factor conditional logistic regression p value of less than 0.05 were then further evaluated by the statistical technique of the Family Based Association Test, using the software program “FBAT”. (FBAT software is provided by the Department of Biostatistics, Harvard School of Public Health, Boston Mass., and is freely downloadable from the web site biosun1.harvard.edu/˜fbat/fbat.htm.) Parameters were set so that the FBAT analysis only included a particular SNP if its frequency amongst affected (“Frequency in Affected”) and unaffected (“Minor Allele Frequency”) siblings combined was greater than or equal to 5%, and if there were at least four informative families. (See, Horvath et al., “Family-based tests for associating haplotypes with general phenotype data: application to asthma genetics”, Genet. Epidemiol., 26:61-69 (2004).) Consequently, only 19 of 25 SNPs of the CFH gene were amenable to FBAT and thus included in Tables 6a and 6d; the remaining six CFH-related SNPs had less than four informative families. In the context of single factor FBAT analysis, an individual SNP was judged to be associated with AMD if it had a p value of less than 0.05.

Results of FBAT analysis conducted without Bonferroni correction are shown in Tables 6(a-c) for the CFH gene (Table 6a), the C3 gene (Table 6b), and the CFD gene (Table 6c), respectively. Each of Tables 6(a-c) sets forth the ‘rs’ identifier of the SNP (column 1), the minor allele (column 2), the number of informative families (column 3), the variance (mean square deviation), S (column 4), the number of standard deviations from the mean, Z (column 5), and the pair-wise (PW) p value (column 6), where. When evaluated without Bonferroni correction, FBAT analysis of individual SNPs revealed a significant association between neovascular AMD and sixteen of the SNPs (twelve in CFH (rs800292, rs35507625, rs572515, rs1061147, rs7529589, rs482934, rs1061170, rs12038333, rs2274700, rs203674, rs375046 and rs16840522), three in C3 (rs2230203, rs406514, and rs423490), and one in CFD (rs1683564),), as shown in Tables 6(a-c), respectively.

A Bonferroni correction was applied to the pair-wise p values that were calculated by FBAT for each allele of the SNPs that met these criteria. By “Bonferroni correction” is meant that, where there are n dependent or independent hypotheses in a given data set, then the statistical significance level for each hypothesis separately is 1/n times what it would be if only one hypothesis were tested. Applying a Bonferroni correction results in a stricter significance threshold. By way of example, to test two independent hypotheses on the same data at a 0.05 significance level, instead of using a p value threshold of 0.05, one would use a stricter threshold of 0.025. Table 6d sets forth the ‘rs’ identifier of the SNP (column 1), the minor allele (column 2), the number of informative families (column 3), the variance, i.e., mean square deviation, S (column 4), the number of standard deviations from the mean, Z (column 5), and the family-wise (FW) p value (column 6). After applying a Bonferroni correction to the FBAT data, only the twelve CFH-related SNPs (rs800292, rs35507625, rs572515, rs1061147, rs7529589, rs482934, rs1061170, rs12038333, rs2274700, rs203674, rs375046 and rs16840522) continued to exhibit a significant association with AMD (Table 6d).

Two of the CFH-related SNPs (rs482934, and rs375046) have not been previously identified as having an association with risk for neovascular AMD.

### Hardy-Weinberg Equilibria:

The frequency of each genotype (homozygous minor, heterozygous, and homozygous major) and allele (minor and major) was calculated for the CFH-related SNPs in the population of affected index cases and, separately, in the population of unaffected siblings (Table 8). The deviation from Hardy-Weinberg equilibrium was tested on each SNP using the chi-square test. No significant deviations from Hardy-Weinberg equilibrium for any of the variants studied were observed in either affected or unaffected siblings, indicating unlikely contamination of the dataset.

### Multiple Conditional Logistic Regression:

To further identify factors associated with AMD, multiple conditional logistic regression (MCLR) analysis was performed on the twelve SNPs of the CFH gene that had survived Bonferroni correction. MCLR analysis was facilitated by the use of the software program SAS (SAS® version 9.1; SAS® Institute Inc, Cary, N.C.) [http://www.sas.com]).

The multiple conditional logistic regression model for each significant SNP in the 1q22-q32 region of chromosome 1 was built using those factors from the single factor model that appeared to be associated with neovascular AMD with a p<0.1. For each significant SNP, the minor allele (in unaffected siblings) in both the homozygous and heterozygous states versus the common allele in the homozygous state was examined in the model. In the context of multiple factor conditional logistic regression analysis, a multiple factor association with AMD was considered significant if it had a p value of less than 0.01.

Results of MCLR are shown in Table 9. For each SNP variant, Table 9 lists the name of the ENSEMBL identifier for the SNP variant, as well as additional known risk factors for AMD (column 1). Additional known risk factors for AMD include a history of smoking (described above), and rs1049331, a polymorphic variant in the HtrA serine peptidase 1 (HTRA1) gene. DeAngelis et al., “Alleles in the HtrA Serine Peptidase 1 Gene Alter the Risk of Neovascular Age-Related Macular Degeneration”, Ophthalmology, 115:1209-1215 (2007). Table 9 also shows the ancestral (or common) allele (column 2), the odds ratio calculated at 95% confidence interval (column 3), and the corresponding p value (column 4). Odds ratios were calculated as the number of index patients having the combination of alleles and/or risk factors shown in column 1, times the number of unaffected subjects having the combination of alleles and/or risk factors shown in column 1, divided by the product of the number of index patients lacking the combination of alleles and/or risk factors shown in column 1 times the number of unaffected subjects lacking the combination of alleles and/or risk factors shown in column 1. Odds ratios were calculated at the 95% confidence interval (“C.I.”).

Consistent with the findings by single factor conditional logistic regression and by FBAT multiple conditional logistic regression confirmed that the CFH-related SNPs rs572515, rs482934, rs2274700, and rs203674 have the most significant association with AMD risk. Of these four CFH-related SNPs, the SNP rs572515 continued to exhibit the most significant association with neovascular AMD according to multiple conditional logistic regression analysis, after controlling for known risk factors.

### Population-Attributable Risk (PAR):

To further investigate the role of these genetic variants on AMD risk, population-attributable risk (PAR) (θ×[RR−1]/RR) was calculated. The relative risk (RR) was approximated by the odds ratio (OR) according to the methods of Armitage et al. (Armitage et al., Statistical Methods in Medical Research, (2^{nd} ed. Oxford, UK, Blackwell Scientific, 1987)) and θ was calculated as the proportion of cases exposed to the factor for each of the four significant SNPs in the 1q22-q32 region, respectively. The highest risk was seen in the CFH-related SNP rs572515, where the presence of two A alleles (two risk alleles) accounts for 33% of the risk in the entire population (adjusted PAR=0.32). When examining the presence of at least one A allele in combination with established risk factors (i.e., smoking), the adjusted PAR is 54% (adjusted PAR=0.54).

### Linkage Disequilibrium:

Linkage disequilibrium plots (r² and D′) were generated using Haploview (http://www.broad.mit.edu/mpg/haploview) for each of the SNPs having a minor allele frequency (MAP) of >5%. (Barrett et al., “Haploview: analysis and visualization of LD and haplotype maps”, Bioinformatics, 21:263-65 (2005)) Each box contains a pair-wise measurement representing either the disequilibrium value D′ (FIGS. 1 and 6) or the correlation coefficient r² (FIGS. 2 and 7). The higher the value of r² or D′, the darker the shading in the box in a continuum, such that when D′ or r²=0, the shading in the box is white; when 0<D′<1, or when 0<r²<1, the shading in the box is shades of grey; when D′ or r²=1, the shading in the box is black. Maximum r² or D′ values of 1.0 occur when there is complete linkage disequilibrium, as indicated by a black box. Linkage disequilibrium is further indicated by the gradient bar above the LD plot.

FIGS. 1 and 2 are linkage disequilibrium plots for CFH-related SNPs. Linkage disequilibrium analysis between the 25 genotyped SNPs in the CFH gene listed in Table 4 revealed three distinct haplotype blocks. Associations between the 25 CFH-related SNPs result in the three distinct haplotype blocks shown in FIGS. 3, 4, and 5, respectively.

FIGS. 6 and 7 are linkage disequilibrium plots for C3-related SNPs. Haplotype association analysis identified six haplotype blocks within the 28 genotyped SNPs of C3 listed in Table 4 (FIGS. 6 and 7, and FIGS. 8 and 9).

A Haploview analysis was also applied to the eight genotyped SNPs of CFD listed in Table 4. Haploview analysis identified two distinct haplotype blocks within the eight CFD-related SNPs, as shown in the linkage disequilibrium plots FIG. 10 (D′) and FIG. 11 (r²).

### Family Based Association Test (“FBAT”) Analysis of Haplotype Blocks

Each haplotype was further tested for association with AMD using FBAT, to determine the best fit for each genotypic model tested (additive, dominant, or recessive). Both CFH and C3 contained significant haplotypes based on FBAT analysis alone. However, after further analysis of these haplotype blocks by conditional logistic regression, only the six haplotypes of the CFH-related SNPs showed significant association with AMD. In order to correct for multiple testing, the permutation test was used to examine each of the resulting haplotypes.

Haplotype analysis of the CFH gene revealed nine novel haplotypes across the three haplotype blocks (FIGS. 3, 4, and 5). As further detailed below, a set of seven of the novel CFH haplotypes were statistically significant for association with AMD under FBAT, an overlapping set of seven of the novel CFH haplotypes were statistically significant for association with AMD under CLR, and six of the novel CFH haplotypes were statistically significant for association with AMD when analyzed by both FBAT and CLR.

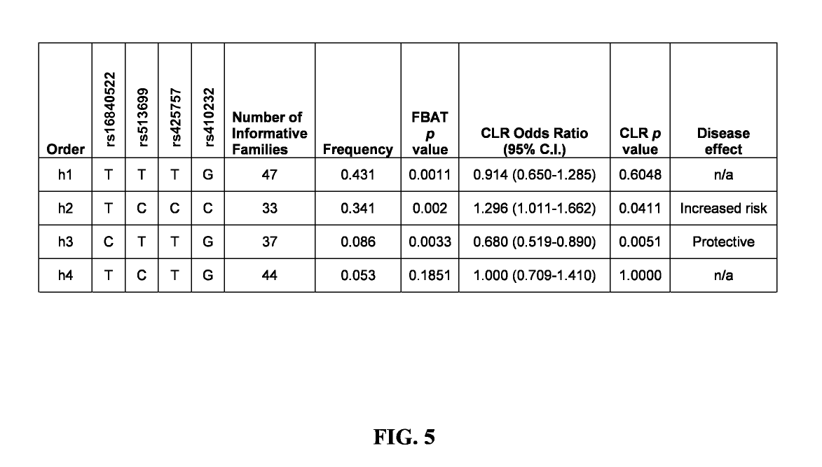

Applying FBAT analysis, seven of these novel CFH haplotypes were statistically significant for association with AMD. Of these seven CFH haplotypes, the most significant according to FBAT analysis was a risk haplotype within CFH Haplotype Block 2, under an additive model (h1, FIG. 4, FBAT p=0.000014). Six additional novel haplotypes were identified as having significant associations with AMD, including a risk associated haplotype in CFH haplotype Block 1 (h1, FIG. 3, FBAT p=0.00034), a protective haplotype in CFH haplotype Block 1 (h2, FIG. 3, FBAT p=0.00064), a protective haplotype in CFH haplotype Block 2 (h2, FIG. 4, FBAT p=0.00014), an associated haplotype in CFH haplotype Block 3 (h1, FIG. 5, FBAT p=0.0011), a risk associated haplotype in CFH haplotype Block 3 (h2, FIG. 5, FBAT p=0.002), and a protective haplotype in CFH haplotype Block 3 (h3, FIG. 5, FBAT p=0.0033).

Permutation testing was conducted to determine the overall significance for all of the haplotypes in each block. In FIG. 3, the estimated CFH haplotypes within CFH Block 1 with allele frequency greater than 0.05 were listed and tested for association. When considering all possible CFH haplotypes of CFH Block 1 together, the resulting p value from 100,000 permutations was 0.000530. In FIG. 4, the estimated CFH haplotypes within CFH Block 2 with allele frequency greater than 0.05 were listed and tested for association. When considering all possible CFH haplotypes of CFH Block 2 together, the resulting p value from 100,000 permutations was 0.000010. In FIG. 5, the estimated CFH haplotypes within CFH Block 3 with allele frequency greater than 0.05 were listed and tested for association. When considering all possible CFH haplotypes of CFH Block 3 together, the resulting p value from 100,000 permutations was 0.005280.

Haplotype analysis of the C3 gene revealed four novel C3 haplotypes across two of the C3 haplotype blocks (FIGS. 8 and 9). One of these novel C3 haplotypes, a risk associated C3 haplotype in C3 haplotype Block 3 (h2, FIG. 8, FBAT p=0.0393), was statistically significant by FBAT for association with AMD under an additive model. One of these novel C3 haplotypes, a risk associated C3 haplotype in C3 haplotype Block 5 (h2, FIG. 9, FBAT p=0.021059), was statistically significant by FBAT for association with AMD under a recessive model.

In FIG. 8, the estimated C3 haplotypes of C3 Block 3 with allele frequency greater than 0.05 were listed and tested for association. When considering all possible C3 haplotypes of C3 Block 3 together, the resulting p value from 100,000 permutations was 0.127100. In FIG. 9, the estimated C3 haplotypes of C3 Block 5 with allele frequency greater than 0.05 were listed and tested for association. When considering all possible C3 haplotypes of C3 Block 5 together, the resulting p value from 100,000 permutations was 0.050753.

### Analysis Of Haplotypes By Conditional Logistic Regression

Whether there was or was not a significant association between each of the haplotypes identified in the LD plots and AMD was tested by conditional logistic regression, with the assistance of SAS® software (SAS® version 9.1; SAS® Institute Inc, Cary, N.C.), but with the modification that each SNP was assigned a score of 0, 1, or 2 according to which of its two alleles was shown to be significant in the haplotype block. The score was assigned based on the number of corresponding alleles in the genotype and the genetic model it was shown to be associated within FBAT (additive, dominant or recessive). According to the haplotypes designated by Haploview and FBAT, the scores were added across the SNPs for each haplotype block. Each score for each block was then analyzed as a continuous variable with respect to AMD status, and controlling for the fact that data was drawn from sibships.

Results of haplotype conditional logistic regression analysis are shown for the CFH gene (FIGS. 3-5) and the C3 gene (FIGS. 8-9). Each of FIGS. 3-5 and 8-9 sets forth the order (as an ‘h’ number identifier) of the haplotype within the block identified in the corresponding LD plot (column 1), the rs identifiers of the SNPs within the haplotype (columns 2+), the corresponding odds ratio (3^{rd} column from right), the corresponding p value of the association (2″ column from right), and the disease effect of the association (far right column). In the context of haplotype conditional logistic regression analysis (CLR), an individual SNP was judged to be associated with AMD if it had a p value of less than 0.05.

After applying a Bonferroni correction to the haplotype FBAT data, the CFH haplotypes were confirmed to be associated with risk of neovascular AMD (p<0.0001) under an additive model (FIGS. 3, 4, and 5). No haplotypes of C3-related SNPs survived Bonferroni correction.

The haplotypes were further tested by conditional logistic regression analysis. Only the CFH haplotypes were significant according to CLR. Seven novel significant haplotypes across the three haplotype blocks were identified after applying conditional logistic regression analysis (CLR) (FIGS. 3, 4, and 5). Of these seven haplotypes, the most significant was a protective haplotype within CFH Haplotype Block 2, under an additive model (h2, FIG. 4, CLR p=3.89E-06). Six additional novel haplotypes were identified as having significant associations with AMD, including a risk associated haplotype in CFH haplotype Block 1 (h1, FIG. 3, CLR p=0.0013), a protective haplotype in CFH haplotype Block 1 (h2, FIG. 3, CLR p=0.0008), a risk associated haplotype in CFH haplotype Block 2 (h1, FIG. 4, CLR p=2.33E-05), a protective haplotype in CFH haplotype Block 2 (h3, FIG. 4, CLR p=2.69E-05), a risk associated haplotype in CFH haplotype Block 3 (h2, FIG. 5, CLR p=0.0411), and a protective haplotype in CFH haplotype Block 3 (h3, FIG. 5, CLR p=0.0051).

None of the C3 haplotypes were considered to show a statistically significant association with AMD according to conditional logistic regression analysis.

### Characteristics of Polymorphic Variants:

CFH rs800292

The polymorphic variant rs800292 is a G>A substitution in exon 2 of the CFH gene. The minor allele, A, was identified as having a protective association with AMD (Table 5a). As shown in Table 9, individuals homozygous for the common allele G (GG) of rs800292 have a 7.75-fold (i.e., 1/0.129) higher risk (p<0.1106) of developing neovascular AMD when compared to individuals homozygous for the minor allele A (AA), whereas individuals heterozygous for the minor and common alleles (AG) have a 4.29-fold (i.e., 1/0.233) (p<0.0096) higher risk of developing AMD when compared to individuals homozygous for the ancestral allele G (GG) (Table 9). The risk variant polymorphism rs800292 of CFH is surrounded by the forward nucleotide sequence: AAATACAGAAATTCTGACAGGTTCCTGGTCTGACCAAACATATCCAGAAGGCACCCAG GCTATCTATAAATGCCGCCCTGGATATAGATCTCTTGGAAAT[X=G/A]TAATAATGGTAT GCAGGAAGGGAGAATGGGTTGCTCTTAATCCATTAAGGAAATGTCAGAGTAAGTACTT AATACATTTGTGAAATTTATGAAAACTAGG (SEQ ID NO: 1), wherein X is a guanine to adenine substitution, guanine being the risk and common allele and adenine being the protective variant and minor allele. Alternatively, the surrounding reverse sequences are: CCTAGTTTTCATAAATTTCACAAATGTATTAAGTACTTACTCTGACATTTCCTTAATGGA TTAAGAGCAACCCATTCTCCCTTCCTGCATACCATTATTA[X=C/T]ATTTCCAAGAGATCT ATATCCAGGGCGGCATTTATAGATAGCCTGGGTGCCTTCTGGATATGTTTGGTCAGACC AGGAACCTGTCAGAATTTCTGTATTT (SEQ ID NO: 2), wherein X is a cytosine to a thymine substitution, cytosine being the common and risk allele, and cytosine being the minor allele and protective variant.

CFH rs35507625

The polymorphic variant rs35507625 is a TT double base insertion in intron 2 of the CFH gene. The rs35507625 polymorphism was identified as having a protective association with AMD. Individuals homozygous for lacking the TT insertion (--, --) of rs35507625 have a 3.04-fold (i.e., 1/0.329) higher risk of developing AMD (p<0.3513) when compared to individuals homozygous for the minor allele TT (TT, TT), whereas individuals heterozygous for the common and minor alleles (TT, --) have a 3.57-fold (i.e., 1/0.280) higher risk of developing neovascular AMD (p<0.0163) when compared to individuals homozygous for the common allele of no insertion. The protective variant polymorphism rs35507625 of CFH is surrounded by the forward nucleotide sequence: GCTGGGCAGGCTGGTCTCGAACTCCTGGCCTCGTGATCCACCCACCTCAGCCTCCCAAA GTGCTGGGATTACAGGCGTGAGCCACCGTGCCCAGCCAATACATCATCATTTTCAAAAA GGGGTGGTCATCCTCCAAAATTAAAAAAGCAAGCATATAGTTTAAGTTCAATTATGAAA TAATGGCTTTGCTATGTTTAATTTTCCTTACATTCAATCTGTCTTCTTATATAATATCAAA TATACTTGTTCCCCCACTCCTACATAAAATATATTCCTTGCTATTACATACTAATTCATA AC[−/TT]TTTTTTTTTCGTTTTAGAAAGGCCCTGTGGACATCCTGGAGATACTCCTTTTGG TACTTTTACCCTTACAGGAGGAAATGTGTTTGAATATGGTGTAAAAGCTGTGTATACAT GTAATGAGGGGTATGTAGTCCATACGAAAAGAGGTTTATAATTAAGATAGTAAATAGG AACTCTACTACTTTATATATTTTTAAGGTTATTATATTTTTCTATGAGCATTTAAAAAAGT AATACACAAGTACCTGAAAGTTTAACTATGATGGAAATAATTAAATCTGGATACCATAT TATCTCCTT (SEQ ID NO: 3), wherein X is a TT insertion, -- being the common and risk allele, and TT being the minor allele and protective variant. Alternatively, the surrounding reverse sequences are:

AAGGAGATAATATGGTATCCAGATTTAATTATTTCCATCATAGTTAAACTTTCAGGTACT TGTGTATTACTTTTTTAAATGCTCATAGAAAAATATAATAACCTTAAAAATATATAAAGT AGTAGAGTTCCTATTTACTATCTTAATTATAAACCTCTTTTCGTATGGACTACATACCCC TCATTACATGTATACACAGCACACCATATTCAAACACATTTCCTCCTGTAAGGGTA AAAGTACCAAAAGGAGTATCTCCAGGATGTCCACAGGGCCTTTCTAAAACGAAAAAAA AA[X=−/AA]GTTATGAATTAGTATGTAATAGCAAGGAATATATTTTATGTAGGAGTGGG GGAACAAGTATATTTGATATTATATAAGAAGACAGATTGAATGTAAGGAAAATTAAAC ATAGCAAAGCCATTATTTCATAATTGAACTTAAACTATATGCTTGCTTTTTTAATTTTGG AGGATGACCACCCCTTTTTGAAAATGATGATGTATTGGCTGGGCACGGTGGCTCACGCC TGTAATCCCAGCACTTTGGGAGGCTGAGGTGGGTGGATCACGAGGCCAGGAGTTCGAG ACCAGCCTGCCCAGC (SEQ ID NO: 4), wherein X is an AA insertion, -- being the common and risk allele, and AA being the protective variant and minor allele.
CFH rs572515

The polymorphic variant rs572515 is a G>A substitution in intron 4 of the CFH gene, which exhibits an increased risk of developing the neovascular form of AMD (Table 9). Individuals homozygous for the A (AA) allele of rs572515 have a 71.095-fold higher risk (p<0.0001) of developing neovascular AMD when compared to individuals homozygous for the ancestral allele G (GG), whereas individuals heterozygous for the risk allele A (AG) have a 3.28-fold (p<0.018) higher risk of developing neovascular AMD when compared to individuals homozygous for the ancestral allele G (GG) (Table 9). The risk variant polymorphism rs572515 of CFH is surrounded by the forward nucleotide sequence:

ATTATTTTAAACACTTAGGTCGAAGAGGATGGAAAATTAACGATGATTTCTCTATTGAA GTTAATGTCATAAAGTTTGCTTTTACATCTTTTAGGAACAC[X=G/A]GTGTTATATTATTCTAGGGCATAAATGAAAATGTATTTAATTATCTCAAGCTTTATATTT CTTAATTATTTAAAAATACTAGTTTGTTACTACAAAATAC (SEQ ID NO: 5) wherein X is a guanine to adenine substitution, guanine being the common allele and adenine being the risk variant and minor allele. Alternatively, the surrounding reverse sequences are:
GTATTTTGTAGTAACAAACTAGTATTTTTAAATAATTAAGAAATATAAAGCTTGAGATA ATTAAATACATTTTCATTTATGCCCTAGAATAATATAACAC[X=C/T]GTGTTCCTAAAAG ATGTAAAAGCAAACTTTATGACATTAACTTCAATAGAGAAATCATCGTTAATTTTCCAT CCTCTTCGACCTAAGTGTTTAAAATAAT (SEQ ID NO: 6) wherein X is a cytosine to a thymine substitution, thymine being the major allele, and cytosine being the minor allele, which is a risk variant.
CFH rs1061147

The polymorphic variant rs1061147 is a C>A substitution in exon 7 of the CFH gene which causes an amino acid codon change of Ala307Ala. rs1061147 is identified herein as being associated with an increased risk of developing AMD. Individuals homozygous for the A (AA) allele of rs1061147 (p<0.0001) have a 20.688-fold higher risk of developing AMD when compared to individuals homozygous for the ancestral allele C(CC), whereas individuals heterozygous for the risk allele A (AC) (p<0.1228) have a 2.051-fold higher risk of developing AMD when compared to individuals homozygous for the ancestral allele C(CC). The risk variant polymorphism rs1061147 of CFH is surrounded by the forward nucleotide sequence: ATGGTGACTACTCACCTTTAAGGATTAAACACAGAACTGGAGATGAAATCACGTACCAG TGTAGAAATGGTTTTTATCCTGCAACCCGGGGAAATACAGC[X=C/A]AAATGCACAAGT ACTGGCTGGATACCTGCTCCGAGATGTACCTGTAAGTTCCATTCATATCTTGACCCATTT CTTAATTCTGAAATTTCTTTTAAACACA (SEQ ID NO: 7), wherein X is a cytosine to adenine substitution, cytosine being the common allele, and adenine being the minor allele and risk variant. Alternatively, the surrounding reverse sequences are:

TGTGTTTAAAAGAAATTTCAGAATTAAGAAATGGGTCAAGATATGAATGGAACTTACAG GTACATCTCGGAGCAGGTATCCAGCCAGTACTTGTGCATTT[X=G/T]GCTGTATTTCCCC GGGTTGCAGGATAAAAACCATITCTACACTGGTACGTGATTTCATCTCCAGTTCTGTGTT TAATCCTTAAAGGTGAGTAGTCACCAT (SEQ ID NO: 8), wherein X is an guanine to a thymine substitution, guanine being the common allele, and thymine being the minor allele and risk variant.
CFH rs7529589

The polymorphic variant rs7529589 is a C>T substitution in intron 7 of the CFH gene. rs7529589 is identified herein as being associated with an increased risk of developing AMD.

Individuals homozygous for the T (TT) allele of rs7529589 (p<0.0001) have a 24.077-fold higher risk of developing AMD when compared to individuals homozygous for the ancestral allele C(CC), whereas individuals heterozygous for the risk allele T (TC) (p<0.1855) have a 1.828-fold higher risk of developing AMD when compared to individuals homozygous for the ancestral allele C(CC). The risk variant polymorphism rs7529589 of CFH is surrounded by the forward nucleotide sequence: AATACATGAGCTAAGCGGTAAAATTGGCATATTTCTCAACCGAAAAGCTTATTTAATTA ACCAGGGATAGGGTATGTGTATTTAAGGGAAATATATTAAA[X=C/T]AGGTCTGTGCATT TTTCTTTCTGGAGAGCCTTCATACAAATTTACGCATCATGTGATCCACAAGACATAATTT CATCTCCATTAACAAAGACCTTCTTGT (SEQ ID NO: 9), wherein X is a cytosine to a thymine substitution, cytosine being the common allele, and thymine being the minor allele and risk variant. Alternatively, the surrounding reverse sequences are:

ACAAGAAGGTCTTTGTTAATGAAGATGAAATTATGTCTTGTGGATCACATGATGCGTAA ATTTGTATGAAGGCTCTCCAGAAAGAAAAATGCACAGACCT[X=G/A]TTTAATATATTTC CCTTAAATACACATACCCTATCCCTGGTTAATTAAATAAGCTTTTCGGTTGAGAAATATG CCAATTTTACCGCTTAGCTCATGTATT (SEQ ID NO: 10), wherein X is an guanine to an adenine substitution, guanine being the common allele, and adenine being the minor allele and risk variant.
CFH rs482934

The polymorphic variant rs482934 is a T>G substitution in intron 7 of the CFH gene, and was identified as being associated with an increased risk of developing AMD. Individuals homozygous for the G (GG) allele of rs482934 (p<0.0002) have a 17.7-fold higher risk of developing neovascular AMD when compared to individuals homozygous for the ancestral allele T (TT), whereas individuals heterozygous for the minor allele G (GT) (p<0.1533) have a 2.15-fold higher risk of developing neovascular AMD when compared to individuals homozygous for the ancestral allele T (TT). The risk variant polymorphism rs482934 of CFH is surrounded by the forward nucleotide sequence:

TGAGTTCTATCATTTGTITTGACCTAGAAACCCTAATGGAATGTGTAATTATACTAAGAA GAGAATATAATTCAGTGATAAAAATTTATCTCTAATATGA[X=T/G]TGTTTATTACAGTA AAATTTCTTTATACTTTTTTTAAAATTTTTATTGCAAGTGAAACCTTGTGATTATCCAGAC ATTAAACATGGAGGTCTATATCATG (SEQ ID NO: 11), wherein X is a thymine to a guanine substitution, thymine being is the common allele, and guanine being the minor allele and risk variant. Alternatively, the surrounding reverse sequences are:
CATGATATAGACCTCCATGTTTAATGTCTGGATAATCACAAGGTTTCACTTGCAATAAA AATTTTIAAAAAAAGTATAAAGAAATTTTACTGTAATAAACA[X=A/C]TCATATTAGAGAT AAATTTTTATCACTGAATTATATTCTCTTCTTAGTATAATTACACATTCCATTAGGGTTTC TAGGTCAAAACAAATGATAGAACTCA (SEQ ID NO: 12), wherein X is an adenine to a cytosine substitution, adenine being the referent allele, and cytosine being the minor allele and risk variant.
CFH rs1061170

The polymorphic variant rs1061170 is a TQC substitution in exon 9 of the CFH gene which causes an amino acid codon change of Tyr402H is. rs1061170 is identified herein as being associated with an increased risk of developing AMD. Individuals homozygous for the C(CC) allele of rs1061170 (p<0.0001) have a 19.291-fold higher risk of developing AMD when compared to individuals homozygous for the ancestral allele T (TT), whereas individuals heterozygous for the risk allele C(CT) (p<0.2754) have a 1.670-fold higher risk of developing AMD when compared to individuals homozygous for the ancestral allele T (TT). The risk variant polymorphism rs1061170 of CFH is surrounded by the forward nucleotide sequence:

GAGCAAATTTATGTTTCTCATTTACTTTATTTATTTATCATTGTTATGGTCCTTAGGAAAA TGTTATTTTCCTTATTTGGAAAATGGATATAATCAAAAT[X=T/C]ATGGAAGAAAGTTTG TACAGGGTAAATCTATAGACGTTGCCTGCCATCCTGGCTACGCTCTTCCAAAAGCGCAG ACCACAGTTACATGTATGGAGAATGG (SEQ ID NO: 13), wherein X is a thymine to a cytosine substitution, thymine being the common allele, and cytosine being the minor allele and risk variant. Alternatively, the surrounding reverse sequences are:
CCATTCTCCATACATGTAACTGTGGTCTGCGCTTTTGGAAGAGCGTAGCCAGGATGGCA GGCAACGTCTATAGATTTACCCTGTACAAACTTTCTTCCAT[X=G/A]ATTTTGATTATATC CATTTTCCAAATAAGGAAAATAACATTTTCCTAAGGACCATAACAATGATAAATAAATA AAGTAAATGAGAAACATAAATITGCTC (SEQ ID NO: 14), wherein X is a guanine to an adenine substitution, guanine being the common allele, and adenine being the minor allele and risk variant.
CFH rs12038333

The polymorphic variant rs12038333 is an A>G substitution in intron 9 of the CFH gene. rs12038333 is identified herein as being associated with an increased risk of developing AMD. Individuals homozygous for the G (GG) allele of rs12038333 (p<0.0001) have a 28.500-fold higher risk of developing AMD when compared to individuals homozygous for the ancestral allele A (AA), whereas individuals heterozygous for the risk allele G (GA) (p<0.1161) have a 2.114-fold higher risk of developing AMD when compared to individuals homozygous for the ancestral allele A (AA). The risk variant polymorphism rs12038333 of CFH is surrounded by the forward nucleotide sequence:

CATCTATGGTAGAACCACCTGGGCCCCCAAAGGCCAATAAAAACGGACATCCCATTGC AGGTCCCAACCAGTGTGCTTACTGCAACAGGGGGGACACTGA[A/G]TGGAAAATTTCTC ATGCCTTACAAAGCCTGATGTTAAACAGTCAGCCTTCTGCCCACCAAATGCCTGGGATA GCTGGGGAGCTTGAGAGAGATACTAAAG (SEQ ID NO: 15), wherein X is an adenine to a guanine substitution, adenine being the common allele, and guanine being the minor allele and risk variant. Alternatively, the surrounding reverse sequences are:
CTTTAGTATCTCTCTCAAGCTCCCCAGCTATCCCAGGCATTTGGTGGGCAGAAGGCTGA CTGTTTAACATCAGGCTTTGTAAGGCATGAGAANITITCCA[X=T/C]TCAGTGTCCCCCCT GTTGCAGTAAGCACACTGGTTGGGACCTGCAATGGGATGTCCGTTTTTATTGGCCTTTGG GGGCCCAGGTGGTTCTACCATAGATG (SEQ ID NO: 16), wherein X is a thymine to cytosine substitution, thymine being the common allele, and cytosine being the minor allele and risk variant.
CFH rs2274700

The polymorphic variant rs2274700 is a G>A substitution in exon 10 of the CFH gene which causes an amino acid codon change of Ala473Ala. rs2274700 is identified herein as having a protective association with AMD. Individuals homozygous for the common allele G (GG) of rs2274700 have a 11.8-fold (i.e., 1/0.085) higher risk of developing neovascular AMD (p<0.0031) when compared to individuals homozygous for the protective, minor allele A (AA). Individuals heterozygous for the protective and common alleles (AG) have a 7.35-fold (i.e., 1/0.136) higher risk of developing neovascular AMD (p<0.0002) when compared to individuals homozygous for the common allele G (GG). The risk variant polymorphism rs2274700 of CFH is surrounded by the forward nucleotide sequence:

TATTCTCTTCCCTTTTAGAAACATGTTCCAAATCAAGTATAGATATTGAGAATGGGTTTA TTTCTGAATCTCAGTATACATATGCCTTAAAAGAAAAAGC[X=G/A]AAATATCAATGCA AACTAGGATATGTAACAGCAGATGGTGAAACATCAGGATCAATTACATGTGGGAAAGA TGGATGGTCAGCTCAACCCACGTGCATTA (SEQ ID NO: 17) wherein X is a guanine to an adenine substitution, guanine being the common and risk allele, and adenine being the minor allele and protective variant. Alternatively, the reverse sequence comprises:
TAATGCACGTGGGTTGAGCTGACCATCCATCTTTCCCACATGTAATTGATCCTGATGTTT CACCATCTGCTGTTACATATCCTAGTTTGCATTGATATTT[X=C/T]GCTTTTTCTTTTAAGG CATATGTATACTGAGATTCAGAAATAAACCCATTCTCAATATCTATACTTGATTTGGAAC ATGTTTCTAAAAGGGAAGAGAATA (SEQ ID NO: 18) wherein X is a cytosine to a thymine substitution, cytosine being the common and risk allele, and thymine being the minor allele and protective variant.
CFH rs203674

The polymorphic variant rs203674 is a T>G substitution in intron 10 of the CFH gene. The rs203674 SNP was identified as being associated with an increased risk of developing the neovascular form of AMD. Individuals homozygous for the G (GG) allele of rs203674 (p<0.0001) have a 31.945-fold higher risk of developing neovascular AMD when compared to individuals homozygous for the ancestral allele T (TT), whereas individuals heterozygous for the risk allele G (GT) (p<0.0602) have a 3.032-fold higher risk of developing neovascular AMD when compared to individuals homozygous for the ancestral allele T (TT). The risk variant polymorphism rs203674 of CFH is surrounded by the forward nucleotide sequence:

TGTTGTGTAGAAAGATATACTTTCACTTTTGACAACTATTTTACGACAACAAATTCTCAC CAGTCATAGATTATTTTTGTACGGTACCTATTTATTAGTA[X=T/G]ATCTAATCAATAAAG CTTTTTCTTCTTAGAATGGGAAATACTCAGATTGTTTATTAGATGACATTAGAAATGACA TTCTAAATTTTTTATGCACTAGAAT (SEQ ID NO: 19) wherein X is a thymine to a guanine substitution, thymine being the common allele, and guanine being the minor allele and risk variant. Alternatively, the reverse sequence of the rs203674 CFH risk variant includes the nucleotide sequence:
ATTCTAGTGCATAAAAAATTTAGAATGTCATTTCTAATGTCATCTAATAAACAATCTGA GTATTTCCCATTCTAAGAAGAAAAAGCTTTATTGATTAGAT[X=A/C]TACTAATAAATAG GTACCGTACAAAAATAATCTATGACTGGTGAGAATTTGTTGTCGTAAAATAGTTGTCAA AAGTGAAAGTATATCTTTCTACACAACA (SEQ ID NO: 20) wherein X is an adenine to a cytosine substitution, adenine being the common allele, and cytosine being the minor allele and risk variant.
CFH rs375046

The polymorphic variant rs375046 is an A>C substitution in intron 15 of the CFH gene. The rs375046 SNP was identified as being associated with an increased risk of developing the neovascular form of AMD. Individuals homozygous for the C(CC) allele of rs375046 (p<0.0002) have a 20.491-fold higher risk of developing neovascular AMD when compared to individuals homozygous for the ancestral allele A (AA), whereas individuals heterozygous for the risk allele C (CA) (p<0.1116) have a 2.455-fold higher risk of developing neovascular AMD when compared to individuals homozygous for the ancestral allele A (AA). The risk variant polymorphism rs375046 of CFH is surrounded by the forward nucleotide sequence:

ACAGTTATTGATCTTTCTATTTTTATTCTATTTTAATCATATAAATTATTTTTCATCAAAAAT TCTAATTTTAATATTTTTATTTTTTATTTTTTATTATAA[X=A/C]ATTAATTATATTTTTAAT ATTTTTTTAGTGGCACAAATACAATTATGCCCACCTCCACCTCAGATTCCCAATTCTCACA ATATGACAACCACACTGAATTA (SEQ ID NO: 21) wherein X is an adenine to a cytosine substitution, adenine being the common allele, and cytosine being the minor allele and risk variant. Alternatively, the reverse sequence of the rs375046 CFH risk variant includes the nucleotide sequence:
TAATTCAGTGTGGTTGTCATATTGTGAGAATTGGGAATCTGAGGTGGAGGTGGGCATAA TTGTATTTGTGCCACTAAAAAATATTAAAAATATAATTAAT[X=T/G]TTATAATAAAAAA TAAAAAATAAAAATATTAAAATTAGAATTTTTGATGAAAAATAATTTATATGATTAAAA TAGAATAAATAGAAAGATCAATAACTGT (SEQ ID NO: 22), wherein X is an thymine to a guanine substitution, thymine being the common allele, and guanine being the minor allele and risk variant.
CFH rs16840522

The polymorphic variant rs16840522 is a T>C substitution in intron 18 of the CFH gene. The minor allele of the rs16840522 SNP was identified as having a protective association with AMD. Individuals heterozygous for the protective allele C (CT) (p<0.0427) have a 2.75 fold reduced risk (i.e., 1/364) of developing neovascular AMD when compared to individuals homozygous for the ancestral allele T (TT). The risk variant polymorphism rs16840522 of CFH is surrounded by the forward nucleotide sequence:

TTTTTAATAGATTTAGAAGAATTTAATGTAATTAAGACAAAATGGCTAATATATTTTCTC AAGTTATAAGAAAAATGTTGTACAGTATTCATTGATTCTA[X=T/C]ATATCGCTATTTTA GAATCCATTACATGTATTGTATGTAACCTATTTTTAAAGATTTGCGGAACAAATACATAT TTTTCCTATTTCAGAAACAGATTGTC (SEQ ID NO: 23) wherein X is a thymine to a cytosine substitution, thymine being the common allele and risk variant, and cytosine being the minor allele and protective variant. Alternatively, the reverse sequence of the rs16840522 CFH risk variant includes the nucleotide sequence:
GACAATCTGTTTCTGAAATAGGAAAAATATGTATTTGTTCCGCAAATCTITAAAAATAG GTTACATACAATACATGTAATGGATTCTAAAATAGCGATAT[X=A/G]TAGAATCAATGA ATACTGTACAACATTTTTCTTATAACTTGAGAAAATATATTAGCCATTTTGTCTTAATTA CATTAAATTCTTCTAAATCTATTAAAAA (SEQ ID NO: 24), wherein X is an adenine to a guanine substitution, adenine being the common allele, and guanine being the minor allele and protective variant.
C3 rs406514

The polymorphic variant rs406514 is a T>C substitution in intron 17 of the C3 gene. The rs406514 SNP was identified as being associated with an increased risk of developing the neovascular form of AMD. The risk variant polymorphism rs406514 of C3 is surrounded by the forward nucleotide sequence:

GGCCTCAAGTGATCCACCTGCCTTGGCCTCCCTAAGTGCTGGGATTTCAGGCATGAGCC ATGGCAACTGGCCTGCTCTGTTCTAAATGCAGATCTAAACC[X=T/C]CCTGCAGGTAACC TGGATGAGGACATCATTGCAGAAGAGAACATCGTTTCCCGAAGTGAGTTCCCAGAGAG CTGGCTGTGGAACGTTGAGGACTTGAAAG (SEQ ID NO: 25) wherein X is a thymine to a cytosine substitution, thymine being the common allele, and cytosine being the minor allele and risk variant. Alternatively, the rs406514 C3 risk variant is surrounded by the reverse nucleotide sequence:
CTTTCAAGTCCTCAACGTTCCACAGCCAGCTCTCTGGGAACTCACTTCGGGAAACGATG TTCTCTTCTGCAATGATGTCCTCATCCAGGTTACCTGCAGG[X=A/G]GGTTTAGATCTGCATTTAGAACAGAGCAGGCCAGTTGCCATGGCTCATGCCTGAAATCC CAGCACTTAGGGAGGCCAAGGCAGGTGGATCACTTGAGGCC (SEQ ID NO: 26) wherein X is an adenine to a cytosine substitution, adenine being the common allele, and cytosine being the minor allele and risk variant.
C3 rs11569450

The polymorphic variant rs11569450 is a C>G substitution in intron 18 of the C3 gene. The rs11569450 SNP was identified as having a protective association with AMD. The protective variant polymorphism rs11569450 of C3 is surrounded by the forward nucleotide sequence:

ATCGTTTCCCGAAGTGAGTTCCCAGAGAGCTGGCTGTGGAACGTTGAGGACTTGAAAGA GCCACCGAAAAATGGGTAAGGCCGGGGTACCCCCGGTACAA[X=C/G]CCACCCCAGAGT CAGACCGTTTAATTTGCATGCACCTGCTATCTCTGGTCTTCTCTGGAATCACAGTGCAAC CCCACAGCCCAACCTAGAAAAACCTGCT (SEQ ID NO: 27) wherein X is a cytosine to a guanine substitution, cytosine being the common allele, and guanine being the minor allele and protective variant. Alternatively, the rs11569450 C3 protective variant is surrounded by the nucleotide sequence:
AGCAGGTTTTTCTAGGTTGGGCTGTGGGGTTGCACTGTGATTCCAGAGAAGACCAGAGA TAGCAGGTGCATGCAAATTAAACGGTCTGACTCTGGGGTGG[X=G/C]TTGTACCGGGGG TACCCCGGCCTTACCCATTTTTCGGTGGCTCTTTCAAGTCCTCAACGTTCCACAGCCAGC TCTCTGGGAACTCACTTCGGGAAACGAT (SEQ ID NO: 28) wherein X is an guanine to an cytosine substitution, guanine being the common allele, and cytosine being the minor allele and protective variant.
C3 rs432823

The polymorphic variant rs432823 is a G>A substitution in intron 18 of the C3 gene. The rs432823 SNP was identified as being associated with an increased risk of developing the neovascular form of AMD. The risk variant polymorphism rs432823 of C3 is surrounded by the forward nucleotide sequence:

ACCTACATGGAGGCACCCCCAGACCCTTCCAGCCTGTCCCTTGGGGTCCCTCTGCACCA GTTCTTCCCCTCTACCACCCTGCTAGATGACATCTCCTAAT[X=G/A]CCCCAACCTCTTCT CCATCCAGAATCTCTACGAAGCTCATGAATATATTTTTGAAAGACTCCATCACCACGTG GGAGATTCTGGCTGTGAGCATGTCGGA (SEQ ID NO: 29) wherein X is a guanine to an adenine substitution, guanine being the common allele, and adenine being the minor allele and risk variant. Alternatively, the rs432823 C3 risk variant is surrounded by the nucleotide sequence:
TCCGACATGCTCACAGCCAGAATCTCCCACGTGGTGATGGAGTCTTTCAAAAATATATT CATGAGCTTCGTAGAGATTCTGGATGGAGAAGAGGTTGGGG[X=C/T]ATTAGGAGATGT CATCTAGCAGGGTGGTAGAGGGGAAGAACTGGTGCAGAGGGACCCCAAGGGACAGGCT GGAAGGGTCTGGGGGTGCCTCCATGTAGGT (SEQ ID NO: 30) wherein X is a cytosine to a thymine substitution, cytosine being the common allele, and adenine being the minor allele and risk variant.
C3 rs366510

The polymorphic variant rs366510 is a A>C substitution in intron 19 of the C3 gene. The rs366510 SNP was identified as being associated with an increased risk of developing the neovascular form of AMD. The risk variant polymorphism rs366510 of C3 is surrounded by the forward nucleotide sequence:

CTTCAGCAAAGACAGGCTGAGTTAGGGAGAGCTCCTGCGGAGTGGACTAAGAGCTGAG ACCCAGGAGCCTGGCCTTGTCCACTCCCCGACCTTGACACTC[X=A/C]GTGTTCTGTCTCT GCCCGAGCAGGGATCTGTGTGGCAGACCCCTTCGAGGTCACAGTAATGCAGGACTTCTT CATCGACCTGCGGCTACCCTACTCTGTT (SEQ ID NO: 31) wherein X is an adenine to a cytosine substitution, adenine being the common allele, and cytosine being the minor allele and risk variant. Alternatively, the rs366510 C3 risk variant is surrounded by the nucleotide sequence:
AACAGAGTAGGGTAGCCGCAGGTCGATGAAGAAGTCCTGCATTACTGTGACCTCGAAG GGGTCTGCCACACAGATCCCTGCTCGGGCAGAGACAGAACAC[X=T/G]GAGTGTCAAGG TCGGGGAGTGGACAAGGCCAGGCTCCTGGGTCTCAGCTCTTAGTCCACTCCGCAGGAGC TCTCCCTAACTCAGCCTGTCTTTGCTGAAG (SEQ ID NO: 32) wherein X is a thymine to a guanine substitution, thymine being the common allele, and guanine being the minor allele and risk variant.
C3 rs423490

The polymorphic variant rs423490 is a C>T substitution in exon 21 of the C3 gene, which causes an amino acid codon change of Ala915Ala. The rs423490 SNP was identified as being associated with an increased risk of developing the neovascular form of AMD. The risk variant polymorphism rs423490 of C3 is surrounded by the forward nucleotide sequence:

ACCAGCAGACCGTAACCATCCCCCCCAAGTCCTCGTTGTCCGTTCCATATGTCATCGTGC CGCTAAAGACCGGCCTGCAGGAAGTGGAAGTCAAGGCTGC[X=C/T]GTCTACCATCATT TCATCAGTGACGGTGTCAGGAAGTCCCTGAAGGTCGTGGTGAGTGCTTGGGGCACCCAC AAACCCTTGTCCTTCAGAGAGGGCTCCT (SEQ ID NO: 33) wherein X is a cytosine to a thymine substitution, cytosine being the common allele, and thymine being the minor allele and risk variant. Alternatively, the rs423490 C3 risk variant is surrounded by the nucleotide sequence:
AGGAGCCCTCTCTGAAGGACAAGGGTTTGTGGGTGCCCCAAGCACTCACCACGACCTTC AGGGACTTCCTGACACCGTCACTGATGAAATGATGGTAGAC[X=G/A]GCAGCCTTGACT TCCACTTCCTGCAGGCCGGTCTTTAGCGGCACGATGACATATGGAACGGACAACGAGG ACTTGGGGGGGATGGTTACGGTCTGCTGGT (SEQ ID NO: 34) wherein X is a guanine to a adenine substitution, guanine being the common allele, and adenine being the minor allele and risk variant.
C3 rs7951

The polymorphic variant rs7951 is a C>T substitution in exon 35 of the C3 gene which causes an amino acid codon change of Ala1437Ala. The rs7951 SNP was identified as having a protective association with AMD. The protective variant polymorphism rs7951 of C3 is surrounded by the forward nucleotide sequence:

GGGAGAGAGGGGTCCAGGAGGGATTTTTCACAGGCTCCACCTTTCCCCAGCTGGCCAAT GGTGTTGACAGATACATCTCCAAGTATGAGCTGGACAAAGC[X=C/T]TTCTCCGATAGGA ACACCCTCATCATCTACCTGGACAAGGTAAGGCTGCATCATCCTCCCCTGGGAGGCTTC CAGGGGCACCCTGACCTCTATCTGGCTG (SEQ ID NO: 35) wherein X is a cytosine to a thymine substitution, cytosine being the common allele, and thymine being the minor allele and protective variant. Alternatively, the rs7951 C3 protective variant is surrounded by the nucleotide sequence:
CAGCCAGATAGAGGTCAGGGTGCCCCTGGAAGCCTCCCAGGGGAGGATGATGCAGCCT TACCTTGTCCAGGTAGATGATGAGGGTGTTCCTATCGGAGAA[X=G/A]GCTTTGTCCAGC TCATACTTGGAGATGTATCTGTCAACACCATTGGCCAGCTGGGGAAAGGTGGAGCCTGT GAAAAATCCCTCCTGGACCCCTCTCTCCC (SEQ ID NO: 36) wherein X is an guanine to an adenine substitution, guanine being the common allele, and adenine being the minor allele and protective variant:
C3 rs2277984

The polymorphic variant rs2277984 is an A>G substitution in intron 36 of the C3 gene. The rs2277984 SNP was identified as being associated with an increased risk of developing the neovascular form of AMD. The risk variant polymorphism rs2277984 of C3 is surrounded by the forward nucleotide sequence:

CATCTCCAGGTCTGAAGACTGAGAACTGGGGATTTGGGCCTCCCAGGCGTGGTCTTMG AGGGAGGCCCTTATCCTCTCATCTTCACATCACATCTGCCC[X=A/G]CAGAGGAAAGCTG TACCCGGTTCTACCATCCGGAAAAGGAGGATGGAAAGCTGAACAAGCTCTGCCGTGAT GAACTGTGCCGCTGTGCTGAGGGTGAGTT (SEQ ID NO: 37) wherein X is an adenine to a guanine substitution, adenine being the common allele, and guanine being the minor allele and risk variant. Alternatively, the rs2277984 C3 risk variant is surrounded by the nucleotide sequence:
AACTCACCCTCAGCACAGCGGCACAGTTCATCACGGCAGAGCTTGTTCAGCTTTCCATC CTCCTTTTTCCGGATGGTAGAACCGGGTACAGCTTTCCTCTG[X=T/C]GGGCAGATGTGAT GTGAAGATGAGAGGATAAGGGCCTCCCTCCAAAGACCACGCCTGGGAGGCCCAAATCC CCAGTTCTCAGTCTTCAGACCTGGAGATG (SEQ ID NO: 38) wherein X is a thymine to a cytosine substitution, thymine being the common allele, and cytosine being the minor allele and risk variant.
C3 rs344555

The polymorphic variant rs344555 is a G>A substitution in intron 37 of the C3 gene. The rs344555 SNP was identified as being associated with an increased risk of developing the neovascular form of AMD. The risk variant polymorphism rs344555 of C3 is surrounded by the forward nucleotide sequence:

CTGAACAAGCTCTGCCGTGATGAACTGTGCCGCTGTGCTGAGGGTGAGTTCCCTGGAGC CGGGAACAGGTGGGTCTGAGCAAGCCACACTTACCCAGGTC[X=G/A]TCTATCCCATGG TCAGGGACCCCCAGACCCATACCCAGGGGATACCAAGGGGGGTAGGCTCCCAGGGCTG GCCACACCCATGGGCAGTAGGCCCCAGATA (SEQ ID NO: 39) wherein X is a guanine to an adenine substitution, guanine being the common allele, and adenine being the minor allele and risk variant. Alternatively, the rs344555 C3 risk variant is surrounded by the nucleotide sequence:
TATCTGGGGCCTACTGCCCATGGGTGTGGCCAGCCCTGGGAGCCTACCCCCCTTGGTAT CCCCTGGGTATGGGTCTGGGGGTCCCTGACCATGGGATAGA[X=C/T]GACCTGGGTAAG TGTGGCTTGCTCAGACCCACCTGTTCCCGGCTCCAGGGAACTCACCCTCAGCACAGCGG CACAGTTCATCACGGCAGAGCTTGTTCAG (SEQ ID NO: 40) wherein X is a cytosine to a thymine substitution, cytosine being the common allele, and adenine being the minor allele and risk variant.
C3 rs11569565

The polymorphic variant rs11569565 is a C>T substitution in intron 38 of the C3 gene. The rs11569565 SNP was identified as having a protective association with AMD. The protective variant polymorphism rs11569565 of C3 is surrounded by the forward nucleotide sequence:

ACTTTCTGCTTGGGAGAGAGAGCCACATGCCCGGGTGCACTTGCAAACCAGGGTGCCCC TCATGGTCAACCTAGCCCACCACCCAAACTGTCTGCCTCTC[X=C/T]CCCACAGTGTACA AGACCCGACTGGTCAAGGTTCAGCTGTCCAATGACTTTGACGAGTACATCATGGCCATT GAGCAGACCATCAAGTCAGGTCAGGCTC (SEQ ID NO: 41) wherein X is a cytosine to a thymine substitution, cytosine being the common allele, and thymine being the minor allele and protective variant. Alternatively, the rs11569565 C3 protective variant is surrounded by the nucleotide sequence:
GAGCCTGACCTGACTTGATGGTCTGCTCAATGGCCATGATGTACTCGTCAAAGTCATTG GACAGCTGAACCTTGACCAGTCGGGTCTTGTACACTGTGGG[X=G/A]GAGAGGCAGACA GTTTGGGTGGTGGGCTAGGTTGACCATGAGGGGCACCCTGGTTTGCAAGTGCACCCGGG CATGTGGCTCTCTCTCCCAAGCAGAAAGT (SEQ ID NO: 42) wherein X is an guanine to an adenine substitution, guanine being the common allele, and adenine being the minor allele and protective variant.

### CFD 1683564

The polymorphic variant rs1683564 is a C>A substitution in the promoter of the C3 gene. The rs1683564 SNP was identified as being associated with an increased risk of developing the neovascular form of AMD. The risk variant polymorphism rs1683564 of C3 is surrounded by the forward nucleotide sequence:

AGTTTGTCTGGGTTTTCCTGTCCCTTGTAGCCAGTCTTCCCAGCTGCCTCTGCCTTGGCCT TTCCAACTTTCTATTCGTCAGGGTGTCAAGCCCCAGAGG[X=C/A]CACAGGGACAGAGA CAAAGGTGTCAAGTGACCTTCCTACCCCCAACAGACCTTCTCTCTAATATAGAGGAGGA AAAGAAGGGAGGGTAGCAGGACAAGAA (SEQ ID NO: 43) wherein X is a cytosine to an adenine substitution, cytosine being the common allele, and adenine being the minor allele and risk variant. Alternatively, the rs1683564 C3 risk variant is surrounded by the nucleotide sequence:
TTCTTGTCCTGCTACCCTCCCTTCTTTTCCTCCTCTATATTAGAGAGAAGGTCTGTTGGGG GTAGGAAGGTCACTTGACACCTTTGTCTCTGTCCCTGTG[X=G/T]CCTCTGGGGCTTGAC ACCCTGACGAATAGAAAGTTGGAAAGGCCAAGGCAGAGGCAGCTGGGAAGACTGGCTA CAAGGGACAGGAAAACCCAGACAAACT (SEQ ID NO: 44) wherein X is a guanine to an thymine substitution, guanine being the common allele, and thymine being the minor allele and risk variant.

### Detection of Allelic Variants in an Individual

The presence of a protective variant or a risk variant in an individual can be determined by standard nucleic acid detection assays including, for example, conventional SNP detection assays, which may include, for example any one or more of amplification-based assays, probe hybridization assays, restriction fragment length polymorphism assays, and direct nucleic acid sequencing. Exemplary protocols for preparing and analyzing samples of interest are discussed in the following sections.

Preparation of Samples for Analysis

Polymorphisms can be detected in a target nucleic acid sample from an individual under investigation. In general, genomic DNA can be analyzed, which can be selected from any biological sample that contains genomic DNA or RNA. For example, genomic DNA can be obtained from peripheral blood leukocytes using standard approaches (QIAamp DNA Blood Maxi kit, Qiagen, Valencia, Calif.). Nucleic acids can be harvested from other biological samples, for example, cells in saliva, cheek scrapings, skin or tissue biopsies, or amniotic fluid as known to those skilled in the art. Methods for purifying nucleic acids from biological samples suitable for use in diagnostic or other assays are known to those of ordinary skill in the art.

Detection of Polymorphisms in Target Nucleic Acids

The identity of bases present at the polymorphic site can be determined in an individual using any of several methods known in the art. Polymorphisms can be detected by direct sequencing, amplification-based assays, probe hybridization-based assays, restriction fragment length polymorphism assays, denaturing gradient gel electrophoresis, single-strand conformation polymorphism analyses, and denaturing high performance liquid chromatography. Other methods to detect nucleic acid polymorphisms include the use of: Molecular Beacons (see, e.g., Piatek et al., Nat. Biotechnol. 16:359-63 (1998)); Tyagi and Kramer, Nat. Biotechnol. 14:303-308 (1996); and Tyagi et al, Nat. Biotechnol. 16:49-53 (1998), the Invader assay (see, e.g., Neri et al., ADv. Nucl. Acid Protein Analysis 3826: 117-125 (2000) and U.S. Pat. No. 6,706,471), and the Scorpion assay (Thelwell et al., Nucl. Acids Res. 28:3752-3761 (2000) and Solinas et al., Nucl. Acids Res. 29:20 (2001).

The design and use of allele-specific probes for analyzing polymorphisms are described, for example, in EP 235,726, and WO 89/11548. Briefly, allele-specific probes are designed to hybridize to a segment of target DNA from one individual but not to the corresponding segment from another individual, if the two segments represent different polymorphic forms. Hybridization conditions are chosen that are sufficiently stringent so that a given probe essentially hybridizes to only one of two alleles. Typically, allele-specific probes are designed to hybridize to a segment of target DNA such that the polymorphic site aligns with a central position of the probe.

The primers, once selected, can be used in standard PCR protocols in conjunction with another common primer that hybridizes to the upstream non-coding strand of the gene at a specified location upstream from the polymorphism (or to the upstream non-coding strand of the gene at a specific location upstream from the polymorphism). The common primers are chosen such that the resulting PCR products can vary from about 100 to about 300 bases in length, or about 150 to about 250 bases in length, although smaller (about 50 to about 100 bases in length) or larger (about 300 to about 500 bases in length) PCR products are possible. The length of the primers can vary from about 10 to 30 bases in length, or about 15 to 25 bases in length.

Many of the methods for detecting polymorphisms involve amplifying DNA or RNA from target samples (e.g., amplifying the segments of the gene of an individual using gene-specific primers and analyzing the amplified gene segments. This can be accomplished by standard polymerase chain reaction (PCR & RT-PCR) protocols or other methods known in the art. Amplification products generated using PCR can be analyzed by the use of denaturing gradient gel electrophoresis. Different alleles can be identified based on sequence-dependent melting properties and electrophoretic migration in solution. See, Erlich, ed., PCR Technology, Principles and Applications for DNA Amplification, Chapter 7 (W.H. Freeman and Co, New York, 1992).

SNP detection can be accomplished by direct PCR amplification, for example, via Allele-Specific PCR (AS-PCR) which is the selective PCR amplification of one of the alleles to detect a Single Nucleotide Polymorphism (SNP). Selective amplification is usually achieved by designing a primer such that the primer will match or mismatch one of the alleles at the 3′-end of the primer. The amplification may result in the generation of allele-specific oligonucleotides, which span any of the SNPs. The CFH-specific-primer sequences and allele-specific oligonucleotides may be derived from the coding (exons) or non-coding (promoter, 5′ untranslated, introns or 3′ untranslated) regions of the gene.

Direct sequencing analysis of polymorphisms can be accomplished using DNA sequencing procedures known in the art. See, Sambrook et al., Molecular Cloning, A Laboratory Manual (2nd Ed., Cold Spring Harbor Laboratories, New York 1989) and Zyskind et al., Recombinant DNA Laboratory Manual (Acad. Press, 1988).

A wide variety of other methods are known in the art for detecting polymorphisms in a biological sample. See, e.g., U.S. Pat. No. 6,632,606; Shi, Am. J. Pharmacogenomics 2:197-205 (2002); Kwok et al., Curr. Issues Biol. 5:43-60 (2003)). Detection of the single nucleotide polymorphic form alone and/or in combination with each other and/or in combination with additional CFH gene polymorphisms may increase the probability of an accurate diagnosis. In one embodiment, screening involves determining the presence or absence of the variant at allele.

In diagnostic methods, the analysis of the SNPs listed herein can be combined with each other and/or can be combined with analysis of polymorphisms in other genes associated with AMD, detection of protein markers of AMD (see, e.g., U.S. Patent Application Publication Nos. US2003/0017501 and US2002/0102581 and International Application Publication Nos. WO0184149 and WO0106262), assessment of other risk factors of AMD (such as family history), with ophthalmological examination, and with other assays and procedures.

Without limiting the above, screening can involve determining whether an individual possesses a haplotype of interest by detecting whether that individual possesses each of the allelic variants known to be embodied in the particular haplotype.

### Treatment of Neovascular AMD

In another aspect, the invention provides a method of treating, slowing the progression of, or reversing the development of age-related macular degeneration in a subject, for example, a human subject. The method comprises (i) reducing the expression of the CFH gene or (ii) reducing the biological activity of the CFH gene product.

The expression of the CFH gene can be reduced by administering to the subject an amount of an agent effective to reduce the expression of the CFH gene. Examples include, for example, an anti-sense polynucleotide or a siRNA effective to reduce the expression of the CFH gene. Specific examples include, for example, siRNA (1900si) (Chien et al., J. Clin. Invest. 116(7):1994-2004 (2006)), sc-60083 (Santa Cruz Biotechnology, Inc., Santa Cruz, Calif.), and sc-43854 (Santa Cruz Biotechnology, Inc., Santa Cruz, Calif.).

Alternatively, the biological activity of the CFH gene product can be reduced, for example, by administering to the subject an effective amount of an agent that binds to the CFH gene product to reduce the activity of the CFH gene product. Exemplary compounds include proteins, for example, antibodies that bind to the CFH gene product. Exemplary proteins include, for example, an anti-CFH antibody. The term antibody is understood to mean an intact antibody, an antigen binding fragment thereof (for example, Fab, Fab′ and (Fab′ h fragments) and single chain antibody binding sites or sFvs.

Selective CFH antagonists can also include peptides and peptide derivatives, which may be administered systemically or locally to the mammal. Other useful selective CFH antagonists include, for example, deoxyribonucleic acids (for example, antisense oligonucleotides), ribonucleic acids (for example, antisense oligonucleotides, aptamers, and interfering RNA) and peptidyl nucleic acids, which once administered reduce or eliminate the expression of certain genes (such as the CFH gene) or can bind to and reduce or eliminate the activity of a target protein or receptor as in the case of aptamers. Other useful selective CFH antagonists include small organic or inorganic molecules that reduce or eliminate the activity when administered to the mammal. Examples include, for example, NVP-LBG976 (Novartis, Basel), and 1-{3-cyclohexyl-2-[(naphthalene-2-carbonyl)-amino]-propionyl}-pyrrolidine-2-carboxylic acid [5-(3-cyclohexyl-ureido)-1-dihydroxyboranyl-pentyl]-amide (Novartis).

Once appropriate selective CFH antagonists have been identified, they may be administered to a mammal of interest (such as a human) in anyone of a wide variety of ways. It is contemplated that a selective CFH antagonist can be administered either alone or in combination with two, three, four or more different selective CFH antagonists either together or one after the other. Although the optimal mode of administration of a particular selective CFH antagonist or combination of selective CFH antagonists can be determined empirically, it is contemplated that selective CFH antagonists may be administered locally or systemically.

Systemic modes of administration include both oral and parenteral routes. Parenteral routes include, for example, intravenous, intrarterial, intramuscular, intradermal, subcutaneous, intranasal, and intraperitoneal routes. It is contemplated that selective CFH antagonists administered systemically may be modified or formulated to target the selective CFH antagonist to the eye. Local modes of administration include, for example, intraocular, intraorbital, subconjuctival, intravitreal, subretinal or transcleral routes. It is noted, however, that local routes of administration are preferred over systemic routes because significantly smaller amounts of the selective CFH antagonist can exert an effect when administered locally (for example, intravitreally) versus when administered systemically (for example, intravenously). Furthermore, the local modes of administration can reduce or eliminate the incidence of potentially toxic side effects that may occur when therapeutically effective amounts of a selective CFH antagonist (i.e., an amount of a selective CFH antagonist sufficient to reduce (for example, by 20%, 30%, 40%, 50%, 60%, 70%, 80%, 90%, or 95%) the biological activity or expression of CFH) are administered systemically.

Administration may be provided as a periodic bolus (for example, intravenously or intravitreally) or as continuous infusion from an internal reservoir (for example, from an implant disposed at an intra- or extra-ocular location (see, U.S. Pat. Nos. 5,443,505 and 5,766,242)) or from an external reservoir (for example, from an intravenous bag). The selective CFH antagonist may be administered locally, for example, by continuous release from a sustained release drug delivery device immobilized to an inner wall of the eye or via targeted transscleral controlled release into the choroid (see, for example, PCT/US00/00207, PCT/US02/14279, Ambati et al., Invest. Qphthalmol. Vis. Sci. 41:1181-1185 (2000) and Ambati et al., Invest. Qphthalmol. Vis. Sci. 41:1186-1191 (2000)). A variety of devices suitable for administering a selective CFH antagonist locally to the inside of the eye are known in the art. See, for example, U.S. Pat. Nos. 6,251,090, 6,299,895, 6,416,777, 6,413,540, and 6,375,972, and PCT/US00/28187.

The selective CFH antagonist may also be administered in a pharmaceutically acceptable carrier or vehicle so that administration does not otherwise adversely affect the recipient's electrolyte and/or volume balance. The carrier may comprise, for example, physiologic saline or other buffer system.

In addition, it is contemplated that the selective CFH antagonist may be formulated so as to permit release of the selective CFH antagonist over a prolonged period of time. A release system can include a matrix of a biodegradable material or a material which releases the incorporated selective CFH antagonist by diffusion. The selective CFH antagonist can be homogeneously or heterogeneously distributed within the release system. A variety of release systems may be useful in the practice of the invention; however, the choice of the appropriate system will depend upon the rate of release required by a particular drug regime. Both nondegradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that selective CFH antagonists having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, nondegradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

One of the primary vehicles currently being developed for the delivery of ocular pharmacological agents is the poly(lactide-co-glycolide) microsphere for intraocular injection. The microspheres are composed of a polymer of lactic acid and glycolic acid, which are structured to form hollow spheres. These spheres can be approximately 15-30 μm in diameter and can be loaded with a variety of compounds varying in size from simple molecules to high molecular weight proteins such as antibodies. The biocompatibility of these microspheres is well established (see, Sintzel et al., Eur. J. Pharm. Biopharm. 42: 358-372 (1996)), and microspheres have been used to deliver a wide variety of pharmacological agents in numerous biological systems. After injection, poly(lactide-co-glycolide) microspheres are hydrolyzed by the surrounding tissues, which cause the release of the contents of the microspheres (Zhu et al., Nat. Biotech. 18: 52-57 (2000)). As will be appreciated, the in vivo half-life of a microsphere can be adjusted depending on the specific needs of the system.

The type and amount of selective CFH antagonist administered may depend upon various factors including, for example, the age, weight, gender, and health of the individual to be treated, as well as the type and/or severity of glaucoma to be treated. As with the modes of administration, it is contemplated that the optimal selective CFH antagonists and dosages of those selective CFH antagonists may be determined empirically.

By way of example, protein-, peptide- or nucleic acid-based selective CFH antagonists can be administered at doses ranging, for example, from about 0.001 to about 500 mg/kg, optionally from about 0.01 to about 250 mg/kg, and optionally from about 0.1 to about 100 mg/kg. Nucleic acid-based selective CFH antagonists may be administered at doses ranging from about 1 to about 20 mg/kg daily. Furthermore, antibodies that are selective CFH antagonists may be administered intravenously at doses ranging from about 0.1 to about 5 mg/kg once every two to four weeks. With regard to intravitreal administration, the selective CFH antagonists, for example, antibodies, may be administered periodically as boluses in dosages ranging from about 10 μg to about 5 mg/eye, and optionally from about 100 μg to about 2 mg/eye. With regard to transcleral administration, the selective CFH antagonists may be administered periodically as boluses in dosages ranging from about 0.1 μg (microgram) to about 1 mg/eye, and optionally from about 0.5 μg (microgram) to about 0.5 mg/eye.

The present invention, therefore, includes the use of a selective CFH antagonists in the preparation of a medicament for treating neovascular AMD. The selective CFH antagonist or antagonists may be provided in a kit which optionally may comprise a package insert with instructions for how to treat the patient with, or at risk of developing, neovascular AMD. For each administration, the selective CFH antagonist may be provided in unit-dosage or multiple-dosage form.

In light of the foregoing description, the specific non-limiting examples presented below are for illustrative purposes and not intended to limit the scope of the invention in any way.

### Tables

TABLE 1
Age Characteristics of EDSP Cohort Population

| | Mean Age | Age Range | Standard | % Male |
|---|---|---|---|---|
| Population | (years) | (years) | Deviation | (n/134) |
| Affected siblings | 72.0 | 48.97-86.49 | 8.1 | 45.5% (61) |
| Unaffected siblings | 75.8 | 50.34-93.92 | 7.9 | 39.6% (53) |

TABLE 2a
Primers Used To Amplify the C1QA, C1QB, and C1QC Genes

| Gene | Region | Forward Primer | Reverse Primer |
|---|---|---|---|
| C1QA | Promoter + | AAGATTCCTTCCGCTCTGGT | CCCCATCCCTGTCCTAGAAT |
| | Exon 1 | (SEQ ID NO: 45) | (SEQ ID NO: 46) |
| C1QA | Exon 2 | GCATGTGTGGATGTGTGTGA | GCAGACGGACTCCTGATTCT |
| | | (SEQ ID NO: 47) | (SEQ ID NO: 48) |
| C1QA | Exon 3a | TGCTTCATTGCCCTTTATCC | CCTGACACCACCTGGAAGAG |
| | | (SEQ ID NO: 49) | (SEQ ID NO: 50) |
| C1QA | Exon 3b | CTGTCCATCGTCTCCTCCTC | GCCTGACTCTTAAGCACTGGA |
| | | (SEQ ID NO: 51) | (SEQ ID NO: 52) |
| C1QC | Promoter | AAATTCTGGGAAAGGGGATG | AAGGAACTGGGAGGGAGAGA |
| | | (SEQ ID NO: 53) | (SEQ ID NO: 54) |
| C1QC | Promoter 1 | GCTCAGCACCCAAAAGAAAG | CCCCAACCCTCTCATTCC |
| | | (SEQ ID NO: 55) | (SEQ ID NO: 56) |
| C1QC | Promoter 2 | GCAACGGTGTACATCCTCCT (SEQ | ACACGGTGTCTGAGTGGTCA |
| | | ID NO: 57) | (SEQ ID NO: 58) |
| C1QC | Exon 1 | ATCCATGGTGAGGCTCCTG (SEQ | GTCAGCCCCAGACAGACACT |
| | | ID NO: 59) | (SEQ ID NO: 60) |
| C1QC | Exon 2a | AATGCCAGCGCTGTGTTC (SEQ ID | ACAGGTTGGCTGTATGCGAC |
| | | NO: 61) | (SEQ ID NO: 62) |
| C1QC | Exon 2b | CAACAGCCTGATCAGATTCAAC | GGTGGGGAGAATGGTCTAGG |
| | | (SEQ ID NO: 63) | (SEQ ID NO: 64) |
| C1QC | Exon 2c | CTGCTCTTCCCCGACTAGG | TGGCTGATGCAAAGTTGAAG |
| | | (SEQ ID NO: 65) | (SEQ ID NO: 66) |
| C1QB | Promoter | AGTAACCTCAGCCCCTCTCC | TGCCAGTTGATGTGTTGTCA |
| | | (SEQ ID NO: 67) | (SEQ ID NO: 68) |
| C1QB | Exon 1 | CGCCACATGATGCTCACT | ACGCAAAGCTCACACTTACG |
| | | (SEQ ID NO: 69) | (SEQ ID NO: 70) |
| C1QB | Exon 2 | ATGGATGCAGATGGAGGAAT | CCAGTGCTTTTGCAGTCTCA |
| | | (SEQ ID NO: 71) | (SEQ ID NO: 72) |
| C1QB | Exon 3a | CCTTCGTTTTACAGATGGGAGA | AGGCATAGTCACAGAAGGTG |
| | | (SEQ ID NO: 73) | A (SEQ ID NO: 74) |
| C1QB | Exon 3b | TGCCCGGTCTCTACTACTTCA | CATGTAACCAACATTTCTGAG |
| | | (SEQ ID NO: 75) | CA (SEQ ID NO: 76) |

TABLE 2b
Primers Used To Amplify the CFH Gene

| Gene | Region | Forward Primer | Reverse Primer |
|---|---|---|---|
| CFH | Promoter 1 | GAGCAACTTGGACACTGCTG | CAATACGCCTCTCAACCCTC |
| | | (SEQ ID NO: 77) | (SEQ ID NO: 78) |
| CFH | Promoter 2 | AACAATGTGTTTGAATGGTCC | AGTGCTTGATGAATTTGGGG |
| | | (SEQ ID NO: 79) | (SEQ ID NO: 80) |
| CFH | Promoter 3 | AAAATCAGCATTTCAATTTGTTG | CCCAATTCTCACTGCACTCC |
| | | (SEQ ID NO: 81) | (SEQ ID NO: 82) |
| CFH | Exon 1 | TGCTGATTTGCACATACCAAG | TCCTGTGAAAAGCATCATTAG |
| | | (SEQ ID NO: 83) | C (SEQ ID NO: 84) |
| CFH | Exon 2 | TTAGATAGACCTGTGACTG | TCAGGCATAATTGCTAC |
| | | (SEQ ID NO: 85) | (SEQ ID NO: 86) |
| CFH | Exon 3 | TCAATTATGAAATAATGGCTTTGC | TGGTATCCAGATTTAATTATTT |
| | (rs35507625) | (SEQ ID NO: 87) | CCA (SEQ ID NO: 88) |
| CFH | Exon 4 | TGCATATGCTGTTCATTTTCC | AAAAAGACTAGATTCCCACTC |
| | | (SEQ ID NO: 89) | TACATT (SEQ ID NO: 90) |
| CFH | Exon 5 | TCCAATCTTATCCTGAGG | AAGTTAAAATGAAATGATGC |
| | | (SEQ ID NO: 91) | (SEQ ID NO: 92) |
| CFH | Exon 6 | GGTTGACTGATTTACCTGATGGA | GAACCTTGAACACAGAAAATG |
| | | (SEQ ID NO: 93) | CT (SEQ ID NO: 94) |
| CFH | Exon 7 | GGACAAATAAATAACACCCACTT | TGCTTCCAACAGCCTTACTTT |
| | | TT (SEQ ID NO: 95) | (SEQ ID NO: 96) |
| CFH | Exon 8 | TCTGAGTTCTATCATTTGTTTTGAC | TCTTTTCTTGGCCCTATTTCTG |
| | (rs482934) | C (SEQ ID NO: 97) | (SEQ ID NO: 98) |
| CFH | Exon 9 | GGTTTCTTCTTGAAAATCACAGG | CCATTGGTAAAACAAGGTGAC |
| | | (SEQ ID NO: 99) | A (SEQ ID NO: 100) |
| CFH | Exon 10 | CTTTTTCTTATTCTCTTCCC | CCCACAAAAAGACTAAAG |
| | | (SEQ ID NO: 101) | (SEQ ID NO: 102) |
| CFH | Exon 11 | GGGAAATACTCAGATTG | GTTTATGTCAAATCAGGAG |
| | | (SEQ ID NO: 103) | (SEQ ID NO: 104) |
| CFH | Exon 12 | TTACAGGGAAAAGGATTTAT | AGATTACAGGCAATGGG |
| | | (SEQ ID NO: 105) | (SEQ ID NO: 106) |
| CFH | Exon 13 | TTGATTGTTTAGGATGC | AAAGTTTATTGTGAAAACAT |
| | | (SEQ ID NO: 107) | (SEQ ID NO: 108) |
| CFH | Exon 14 | TACCAGTGTGTATTGGTAAT | TGGAAATGTTGAGGC |
| | | (SEQ ID NO: 109) | (SEQ ID NO: 110) |
| CFH | Exon 15 | AGTTGGTTTGATTCCTATC | GGAACAGTGTTTTCATTAGT |
| | | (SEQ ID NO: 111) | (SEQ ID NO: 112) |
| CFH | Exon 16 | CAAGCCAAAAGTTCTATTGTTTAA | TTGTTTACACGAAGCACAAGA |
| | (rs375046) | TG (SEQ ID NO: 113) | GA (SEQ ID NO: 114) |
| CFH | Exon 17 | TGGAGGAATATATCTTTGCGAGT | TCAATTATTCCCCTCACTTTGA |
| | | (SEQ ID NO: 115) | (SEQ ID NO: 116) |
| CFH | Exon 18 | GTTGGTGACAGTCCGATAGACA | AATTTCCCACAGCAGTCCAG |
| | | (SEQ ID NO: 117) | (SEQ ID NO: 118) |
| CFH | Exon 19 | AATGTAATTAAGACAAAATGGCT | CGAACTCCTGACCTCAAGT |
| | | A (SEQ ID NO: 119) | (SEQ ID NO: 120) |
| CFH | Exon 20 | CTACTCAAAATGAACACTAGG | ACCCTATTACTTGTGTTCTG |
| | | (SEQ ID NO: 121) | (SEQ ID NO: 122) |
| CFH | Exon 21 | GTGTTTGCGTTTGCC | GAGATTTTTCCAGCCAC |
| | | (SEQ ID NO: 123) | (SEQ ID NO: 124) |
| CFH | Exon 22 | GGTTTGGATAGTGTTTTGAG | ACCGTTAGTTTTCCAGG |
| | | (SEQ ID NO: 125) | (SEQ ID NO: 126) |

TABLE 2c
Primers Used To Amplify the CFI Gene

| Gene | Region | Forward Primer | Reverse Primer |
|---|---|---|---|
| CFI | Promoter | TGATGAACCCAACTGTGTCAG | TTTGGCTGAAATCCAGAGAGA |
| | | (SEQ ID NO: 127) | (SEQ ID NO: 128) |
| CFI | Exon 1 | AAATTCTTTCAGAGTTCAAAAGTA | ATCAAGATATTAACATAAAACA |
| | | CAAAG (SEQ ID NO: 129) | TTTGTTGC (SEQ ID NO: 130) |
| CFI | Exon 2 | CTTGAAGCCACCAGACAACA | TGGCATACAAATACCCTTTTAT |
| | | (SEQ ID NO: 131) | ATCAT (SEQ ID NO: 132) |
| CFI | Exon 3 | TCGTCATGATGTTCAAAGCTC | GGGTTCCAAGTGTTGGGTAA |
| | | (SEQ ID NO: 133) | (SEQ ID NO: 134) |
| CFI | Exon 4 | CTTGCCCAAGCTGTAACTCC | GCAACGAGGCATCAATCAT |
| | | (SEQ ID NO: 135) | (SEQ ID NO: 136) |
| CFI | Exon 5 | TCTTAATGACTCCATTATCCCAAA | GGCATGCTGTGCAAACATAA |
| | | (SEQ ID NO: 137) | (SEQ ID NO: 138) |
| CFI | Exon 6 | TGTGGAGACCAAAGTGATGAA | CACCCATCTATGTTCCCCTTA |
| | | (SEQ ID NO: 139) | (SEQ ID NO: 140) |
| CFI | Exon 7 | GAAATAAGGTGCAATGGTTTTCA | AGAAAAGGCCTCTACGCCTAA |
| | | (SEQ ID NO: 141) | (SEQ ID NO: 142) |
| CFI | Exon 8 | GGGAGGATAAGTTTTAAGGCAGA | CCAAAACTACTTGTTGCTTGAA |
| | | (SEQ ID NO: 143 | TC (SEQ ID NO: 144) |
| CFI | Exon 9 + 10 | TGGCTGTGACTCTCCTGTTTT | TGCTTTATCATCTGCCACAATC |
| | | (SEQ ID NO: 145) | (SEQ ID NO: 146) |
| CFI | Exon 11 | TTCTGGGGAAATGAAAAAGG | GCTTCTCTCTGAGTGCTAGGAA |
| | | (SEQ ID NO: 147) | A (SEQ ID NO: 148) |
| CFI | Exon 12 | CCCTTTCATAATCCCAATGGT | CTACAGCCAGAAGGCCAAAT |
| | | (SEQ ID NO: 149) | (SEQ ID NO: 150) |
| CFI | Exon 13 | AGAGCCCATGCTATTGCTGT | GGCATAAACTCTGTGGAGACC |
| | | (SEQ ID NO: 151) | (SEQ ID NO: 152) |

TABLE 2d
Primers Used To Amplify the C2 Gene

| Gene | Region | Forward Primer | Reverse Primer |
|---|---|---|---|
| C2 | Promoter | TCTGTGGGTCAGGGAGACTAA | GCACCTTGCTCTTGTGTGAA |
| | | (SEQ ID NO: 153) | (SEQ ID NO: 154) |
| C2 | Exon 1 | CCACTTGGTACAACCCAAAA | ACACACAGGACCCTGACGTT |
| | | (SEQ ID NO: 155) | (SEQ ID NO: 156) |
| C2 | Exon 1 + 2 | GCTGCTGACCCAGCTCTAGT | CAGGTTAAGTCCCACCCTGA |
| | | (SEQ ID NO: 157) | (SEQ ID NO: 158) |
| C2 | Exon 3 | TGCCAAAAACAGCAATTTCA | CTATCCCCACAGAGGCTTCC |
| | | (SEQ ID NO: 159) | (SEQ ID NO: 160) |
| C2 | Exon 4 + 5 | GGAACTGGGAAGCTTCTGCT | AAGGTTGCATCCTCCCTTG |
| | | (SEQ ID NO: 161) | (SEQ ID NO: 162) |
| C2 | Exon 6 | TGTTTGAGGTGGGGTTTCTG | GTTCTGAGGAGGGGAAGGAG |
| | | (SEQ ID NO: 163) | (SEQ ID NO: 164) |
| C2 | Exon 7 | TCCAGTTTCTCTGCCTCCTC | ACCATAACCCTGGACGTTGA |
| | | (SEQ ID NO: 165) | (SEQ ID NO: 166) |
| C2 | Exon 8 | CCTAGGTGGTAGGTGGGAAG | GGAGAATCAGAATTCAGGGC |
| | | (SEQ ID NO: 167) | (SEQ ID NO: 168) |
| C2 | Exon 9 | TGAGGTTCCCAGGCTAAATG | CCATGGGCAGTAGTTTGACC |
| | | (SEQ ID NO: 169) | (SEQ ID NO: 170) |
| C2 | Exon 10 + 11 | GACAGCCTCCTGTCTCATGG | GCCGCTTTTACCTCTGGAAT |
| | | (SEQ ID NO: 171) | (SEQ ID NO: 172) |
| C2 | Exon 12 + 13 | ATTCCAGAGGTAAAAGCGGC | GGATCTCCAGAGCACTCTTC |
| | | (SEQ ID NO: 173) | C (SEQ ID NO: 174) |
| C2 | Exon 14 + 15 | CAGGGAAGAGTGCTCTGGA | AGCCAGACCTGGAGAAGGA |
| | | (SEQ ID NO: 175) | (SEQ ID NO: 176) |
| C2 | Exon 16 + 17 | AACCTCCCAAAGTGCTGAGA | CAACCTCCCTAGATCCCTCA |
| | | (SEQ ID NO: 177) | (SEQ ID NO: 178) |
| C2 | Exon 18 | AGGTTGGGGCTTACAGTTGG | CAAGGCCAGCCCTACCTG |
| | | (SEQ ID NO: 179) | (SEQ ID NO: 180) |

TABLE 2e
Primers Used To Amplify the CFB Gene

| Gene | Region | Forward Primer | Reverse Primer |
|---|---|---|---|
| CFB | Promoter 1 | AGTGATCTGCCTGCCTCAAC | TCTCCGCTCAAGGAAAACTG |
| | | (SEQ ID NO: 181) | (SEQ ID NO: 182) |
| CFB | Promoter 2 | GAAGGCCACCTGTGTCTCTG | ATGTGAAAGTCTCGTGGCG |
| | | (SEQ ID NO: 183) | (SEQ ID NO: 184) |
| CFB | Exon 1a | CCCAGCCAGTTCTCTCCTTT | GGGAAATTCCATGTGAAAGT |
| | | (SEQ ID NO: 185) | G (SEQ ID NO: 186) |
| CFB | Exon 1b | CACACAAGCTGGGAAATCCT | CAGCCCCCAGCCTTTTATAC |
| | | (SEQ ID NO: 187) | (SEQ ID NO: 188) |
| CFB | Exon 1c | TGGGACTTCTGCAGTTTCTG | AAAGGCCAAGGAGGGATG |
| | | (SEQ ID NO: 189) | (SEQ ID NO: 190) |
| CFB | Exon 2 | AGCAGACAAGCAAAGCAAGC | GTTTCTGCCTTAGGCCACTG |
| | | (SEQ ID NO: 191) | (SEQ ID NO: 192) |
| CFB | Exon 3 | GAATGCGCTGTTTCTCAGTG | CCCGTGAGCAAGGTAGAGAG |
| | | (SEQ ID NO: 193) | (SEQ ID NO: 194) |
| CFB | Exon 4 | GGCTCCGGACACTGTAACTC | GTGAGGAAAGAGCAGGGTTG |
| | | (SEQ ID NO: 195) | (SEQ ID NO: 196) |
| CFB | Exon 5 + 6 | CCCGTCTCTTTGGTCACTGT | TCAGAGGGGCTGGAATGAT |
| | | (SEQ ID NO: 197) | (SEQ ID NO: 198) |
| CFB | Exon 7 | CTCAGCCCTTGAGCCTCTTA | CTGTCGCCCTCAAGGTAGTC |
| | | (SEQ ID NO: 199) | (SEQ ID NO: 200) |
| CFB | Exon 8 | TGTGGGATTTCAGTTGCAGA | CCCCAACCATGGGTATAGTG |
| | | (SEQ ID NO: 201) | (SEQ ID NO: 202) |
| CFB | Exon 9 + 10 | CCTTCCTCAACTTGCTCACC | CAGGGGAACCTGAGGAGAGT |
| | | (SEQ ID NO: 203) | (SEQ ID NO: 204) |
| CFB | Exon 11 + 12 | GACTCCTACCCAAAAGGCTG | AGAAAGTGGGAGGTGTTGCC |
| | | (SEQ ID NO: 205) | (SEQ ID NO: 206) |
| CFB | Exon 13 + 14 | TGGGGGAAGACGTGAAGTTA | CCTATTGCACCTCCCTCTCA |
| | | (SEQ ID NO: 207) | (SEQ ID NO: 208) |
| CFB | Exon 15 + 17 | AAGGCAATGGGGAGATGAC | ACATGCATTGAGCTTTCCTG |
| | | (SEQ ID NO: 209) | (SEQ ID NO: 210) |
| CFB | Exon 18 | TGAGCTGGGTCCCTAGTCTG | CAGTGCCTGTTCCCCACT |
| | | (SEQ ID NO: 211) | (SEQ ID NO: 212) |

TABLE 2f
Primers Used To Amplify the CFID Gene

| Gene | Region | Forward Primer | Reverse Primer |
|---|---|---|---|
| CFD | Promoter | AGGCTCCACCCAGTTT | AGACCCTCGGGCCTCT |
| | | GTC | GT |
| | | (SEQ ID NO: 213) | (SEQ ID NO: 214) |
| CFD | Exon 1 | GGACTCTGGGTGTGGT | GAGAGACACCCCCTTC |
| | | GAAG | CTG |
| | | (SEQ ID NO: 215) | (SEQ ID NO: 216) |
| CFD | Exon 2 | CCCAGAGATGGGATTC | GCCCAGGAGAACCTGC |
| | | TTGC | AC |
| | | (SEQ ID NO: 217) | (SEQ ID NO: 218) |
| CFD | Exon 3 | CTGGAGGACGCGTGAG | GAGGGTGTGGGGGTTG |
| | | TG | TG |
| | | (SEQ ID NO: 219) | (SEQ ID NO: 220) |
| CFD | Exon 4 | AAATCTCTCCTGCTGC | CTGGGCCCTGTTCCTA |
| | | ACTGA | CTT |
| | | (SEQ ID NO: 221) | (SEQ ID NO: 222) |
| CFD | Exon 5a | AATTAACACGGGAGGG | CCAATTGCCCAGCTAA |
| | | ATGA | TTTT |
| | | (SEQ ID NO: 223) | (SEQ ID NO: 224) |
| CFD | Exon 5b | GGTCACCCAAGCAACA | GGTCAGGCTGATCAGG |
| | | AAGT | AACT |
| | | (SEQ ID NO: 225) | (SEQ ID NO: 226) |

TABLE 2g
Primers Used To Amplify the C3 Gene

| Gene | Region | Forward Primer | Reverse Primer |
|---|---|---|---|
| C3 | Promoter 1 | TGGGAGGAAGACCACCTTTAC | AGCCCAGCACAGAAACTCC |
| | | (SEQ ID NO: 227) | (SEQ ID NO: 228) |
| C3 | Promoter 2 | GAAGTTTTCCCTGACCCTCC | TCCATGGGGTGAGTAACCTG |
| | | (SEQ ID NO: 229) | (SEQ ID NO: 230) |
| C3 | Exon 1 | TGGGTCCAACAGAGAAAGGT | ACCCCCAAATGTCTGCTTC |
| | | (SEQ ID NO: 231) | (SEQ ID NO: 232) |
| C3 | Exon 2 | GGCAGTGCCCAGAGAAGATA | GCAGGGCTTAGAAAGGGAG |
| | | (SEQ ID NO: 233) | A (SEQ ID NO: 234) |
| C3 | Exon 3 + 4 | AAGAATAATGGGCAGGCAAG | TTGCCTCTCCTAAGCCTGTG |
| | | (SEQ ID NO: 235) | (SEQ ID NO: 236) |
| C3 | Exon 5 + 6 | CACCTGGGTCCCTGTTCTT | GGAGAGATGGCGTTGGTG |
| | | (SEQ ID NO: 237) | (SEQ ID NO: 238) |
| C3 | Exon 6 + 7 | CTTGAGGAGGGGGCTCAG | GTCCCCCACCTGGTCTTC |
| | | (SEQ ID NO: 239) | (SEQ ID NO: 240) |
| C3 | Exon 8 + 9 | GTCTTCAGCACAGGCAGGA | GACCTGGTCTCCCCTCTCA |
| | | (SEQ ID NO: 241) | (SEQ ID NO: 242) |
| C3 | Exon 10 + 11 | AAGTTGGGGTCCATTCTGAG | GACAGGACCCCACTGTGC |
| | | (SEQ ID NO: 243) | (SEQ ID NO: 244) |
| C3 | Exon 12 | CACCAATTCCCAGGTCTCAG | GGGTGAGTGGCAGGGAAC |
| | | (SEQ ID NO: 245) | (SEQ ID NO: 246) |
| C3 | Exon 13 | CCTCCTCCTTGTTTCTTCCG | AGACAGTTGAGAGACAGAG |
| | | (SEQ ID NO: 247) | AGGG (SEQ ID NO: 248) |
| C3 | Exon 14 | CCCCCAACCTTTCTGTCTTT | AACCATCCCTGTGTCTGGTC |
| | | (SEQ ID NO: 249) | (SEQ ID NO: 250) |
| C3 | Exon 15 | GAGATGAGAGTGGGACAGGG | TACTAGGTGGTGGTGGAGGC |
| | | (SEQ ID NO: 251) | (SEQ ID NO: 252) |
| C3 | Exon 16 | ATCAAATGGGAGCAGGAACA | CTTGGGGTACTTGCCGACT |
| | | (SEQ ID NO: 253) | (SEQ ID NO: 254) |
| C3 | Exon 17 | GTGCAGGAGCCAGAGAGG | CACCAGACAGGGCATCCT |
| | | (SEQ ID NO: 255) | (SEQ ID NO: 256) |
| C3 | Exon 18 | TGCCCAGCCTTAATTTTTG | GGTTGCACTGTGATTCCAGA |
| | | (SEQ ID NO: 257) | (SEQ ID NO: 258) |
| C3 | Exon 19 | GGTCCCTCTGCACCAGTTC | AACATAGGGTTGATTGAGTT |
| | | (SEQ ID NO: 259) | TGG (SEQ ID NO: 260) |
| C3 | Exon 20 | CCTGCGGAGTGGACTAAGAG | ACCTGCCAGGGAGAGAAAG |
| | | (SEQ ID NO: 261) | G (SEQ ID NO: 262) |
| C3 | Exon 21 | ATCCGAGCCGTTCTCTACAA | CCTGGATCTCCAACCTGAGT |
| | | (SEQ ID NO: 263) | (SEQ ID NO: 264) |
| C3 | Exon 22 + 23 | CCTGCTGACCATCTGTGTGT | CGGGTTAAACACCTCCAGAA |
| | | (SEQ ID NO: 265) | (SEQ ID NO: 266) |
| C3 | Exon 24 | TCCTCGCCTGTCCCTAACC | GGATCTTAGGGGAGGGATGC |
| | | (SEQ ID NO: 267) | (SEQ ID NO: 268) |
| C3 | Exon 25 | GTCCCCTCCTCCCAGAAC | GCTGGCCTAAGGGACCAC |
| | | (SEQ ID NO: 269) | (SEQ ID NO: 270) |
| C3 | Exon 26 | AAGGCAAACCAGGCAAACT | GGGCTCTCTCTCGTGTTCAT |
| | | (SEQ ID NO: 271) | (SEQ ID NO: 272) |
| C3 | Exon 27 | AGCGGGGTAACACCTAGAAG | GAGCTGCAAATTCCCTGAAG |
| | | (SEQ ID NO: 273) | (SEQ ID NO: 274) |
| C3 | Exon 28 | ATGCTCAGGAAGTGCTGCTC | CTAAAAGCCATGCATCCCAG |
| | | (SEQ ID NO: 275) | (SEQ ID NO: 276) |
| C3 | Exon 29 | AGCATGCATCTCTTTCTGAGC | CTGGGCCTCAGTGTCTTCTC |
| | | (SEQ ID NO: 277) | (SEQ ID NO: 278) |
| C3 | Exon 30 + 31 | GATGTCCCAGCTCTGATTTG | CTAGGACTGCTGGGGACAAG |
| | | (SEQ ID NO: 279) | (SEQ ID NO: 280) |
| C3 | Exon 32 + 33 | GGAACAGAAACCCACACCTG | TAGAGGGATGGCCAAGATGA |
| | | (SEQ ID NO: 281) | (SEQ ID NO: 282) |
| C3 | Exon 34 + 35 | CCTGATCAGACCTCCTTGTCC | CTGACCCAGAGACAAAAGCT |
| | | (SEQ ID NO: 283) | G (SEQ ID NO: 284) |
| C3 | Exon 36 | TTCAGTTTCTCCACCACTGC | GACCTTCATTCTCAGATCCCC |
| | | (SEQ ID NO: 285) | (SEQ ID NO: 286) |
| C3 | Exon 37 + 38 | CTGAAGACTGAGAACTGGGG | CCATGACAACCACACCTACC |
| | | (SEQ ID NO: 287) | (SEQ ID NO: 288) |
| C3 | Exon 39 + 40 | TTCTGCTTGGGAGAGAGAGC | GAAGAATGGCAGGTCAGGA |
| | | (SEQ ID NO: 289) | A (SEQ ID NO: 290) |
| C3 | Exon 41 | AGTGAGTGCTTTCCCTGCG | CTGGGGATTTCAGCCTCTC |
| | | (SEQ ID NO: 291) | (SEQ ID NO: 292) |

TABLE 2h
Primers Used To Amplify the C5AR1 Gene

| Gene | Region | Forward Primer | Reverse Primer |
|---|---|---|---|
| C5AR1 | Exon 1 | AAGAGATGGCCCCAAATAGG | ACAGAGTTGACGGCGAGAGT |
| | | (SEQ ID NO: 293) | (SEQ ID NO: 294) |
| C5AR1 | Exon 2a | AGCCAGGATGCCCCCTAC | GGGTTTAAACACCAGCAGAA |
| | | (SEQ ID NO: 295) | AG (SEQ ID NO: 296) |
| C5AR1 | Exon 2b | ATCCTGCCCTCCCTCATC | ATGGCTCCAGGAAGGACATC |
| | | (SEQ ID NO: 297) | (SEQ ID NO: 298) |
| C5AR1 | Exon 2c | CCAAGACACTCAAGGTGGTG | CTGGGAAAGACAGGCAACAT |
| | | (SEQ ID NO: 299) | (SEQ ID NO: 300) |
| C5AR1 | Exon 2d | GGCCATTCTCCCTCTTGTTT | AGCCACTGCTCTCAGCCTAA |
| | | (SEQ ID NO: 301) | (SEQ ID NO: 302) |
| C5AR1 | Exon 2e | GGACACTATGGCCCAGAAGA | GCCACTGCTCTCAGCCTAAT |
| | | (SEQ ID NO: 303) | (SEQ ID NO: 304 |
| C5AR1 | Exon 2f | GGAGGCTCTGTCTCAAAAGC | TCAGCAGTGTGAAAATCGAC |
| | | (SEQ ID NO: 305) | (SEQ ID NO: 306) |
| C5AR1 | Exon 2g | TCAAAAGTTCTTTGGGACAAAAC | CAATTGTGTTGAGTTTGCAAT |
| | | (SEQ ID NO: 307) | G (SEQ ID NO: 308) |

TABLE 3
Chromosomal Location of SNPs

| | Chromo- | | ENSEMBL location |
|---|---|---|---|
| Gene | some | SNP | (bp) |
| | | C1QA (Chromosome 1) | |
| C1QA | 1 | rs12058824 | 22,835,637 |
| C1QA | 1 | rs172378 | 22,838,025 |
| | | C1QC (Chromosome1) | |
| C1QC | 1 | c.-85 | 22,843,019 |
| C1QC | 1 | rs36049190 | 22,846,286 |
| C1QC | 1 | rs9434 | 22,846,884 |
| | | C1QB (Chromosome 1) | |
| C1QB | 1 | rs291990 | 22,852,242 |
| | | CFH (Chromosome 1) | |
| CFH | 1 | rs35906110* | 194,887,792 |
| CFH | 1 | rs800292* | 194,908,856 |
| CFH | 1 | rs35507625* | 194,909,593 |
| CFH | 1 | rs35814900* | 194,909,603 |
| CFH | 1 | rs572515* | 194,912,884 |
| CFH | 1 | rs1061147* | 194,920,947 |
| CFH | 1 | rs7529589* | 194,924,902 |
| CFH | 1 | rs482934* | 194,925,120 |
| CFH | 1 | rs1061170* | 194,925,860 |
| CFH | 1 | rs12038333* | 194,939,077 |
| CFH | 1 | rs2274700* | 194,949,570 |
| CFH | 1 | rs203674* | 194,951,248 |
| CFH | 1 | rs3753396* | 194,962,365 |
| CFH | 1 | rs435628* | 194,972,509 |
| CFH | 1 | rs375046* | 194,972,549 |
| CFH | 1 | rs35292876* | 194,973,265 |
| CFH | 1 | rs515299* | 194,973,300 |
| CFH | 1 | rs1065489* | 194,976,397 |
| CFH | 1 | rs16840522* | 194,977,539 |
| CFH | 1 | rs385543* | 194,977,540 |
| CFH | 1 | rs513699* | 194,979,200 |
| CFH | 1 | rs35274867* | 194,979,219 |
| CFH | 1 | c.3172 * | 194,979,232 |
| CFH | 1 | rs410232* | 194,979,249 |
| CFH | 1 | rs409953* | 194,979,362 |
| | | CFI (Chromosome 4) | |
| CFI | 4 | c.1657* | 110,881,593 |
| CFI | 4 | IVS12 + 96* | 110,882,999 |
| CFI | 4 | IVS12 + 5 * | 110,883,091 |
| CFI | 4 | rs41278049* | 110,886,789 |
| CFI | 4 | rs9998151* | 110,886,794 |
| CFI | 4 | rs41278047* | 110,886,934 |
| CFI | 4 | IVS9 + 17 * | 110,890,087 |
| CFI | 4 | rs5860990* | 110,898,269 |
| CFI | 4 | rs4382037* | 110,901,067 |
| CFI | 4 | IVS3 − 25_27* | 110,902,324 |
| CFI | 4 | rs17610314* | 110,902,402 |
| CFI | 4 | IVS3 + 6 * | 110,905,135 |
| CFI | 4 | c.191 * | 110,907,295 |
| CFI | 4 | rs4382036* | 110,942,466 |
| CFI | 4 | c.-13 * | 110,942,489 |
| CFI | 4 | c.-352 * | 110,942,928 |
| CFI | 4 | rs3840506* | 110,942,940 |
| CFI | 4 | c.-380 * | 110,942,979 |
| CFI | 4 | rs3806820* | 110,943,016 |
| | | C2 (Chromosome 6) | |
| C2 | 6 | rs7746553 | 32,003,952 |
| C2 | 6 | c.386 | 32,004,617 |
| C2 | 6 | rs9332739* | 32,011,783 |
| C2 | 6 | rs9332740 | 32,011,910 |
| C2 | 6 | rs1042663 | 32,013,109 |
| C2 | 6 | rs550605 | 32,015,126 |
| C2 | 6 | rs2242572* | 32,018,908 |
| C2 | 6 | rs547154* | 32,018,917 |
| C2 | 6 | c.*82 | 32,021,105 |
| | | CFB (Chromosome 6) | |
| CFB | 6 | c.-1476 | 32,020,502 |
| CFB | 6 | rs4151667 | 32,022,003 |
| CFB | 6 | rs12614 | 32,022,158 |
| CFB | 6 | rs641153* | 32,022,159 |
| CFB | 6 | rs1048709* | 32,022,914 |
| CFB | 6 | rs4151659 | 32,026,443 |
| CFB | 6 | rs2072633* | 32,027,557 |
| | | CFD (Chromosome 19) | |
| CFD | 19 | rs1683564 | 810,214 |
| CFD | 19 | c.-322 | 810,368 |
| CFD | 19 | rs8107095 | 810,831 |
| CFD | 19 | c.522 | 812,863 |
| CFD | 19 | rs1683561 | 813,024 |
| CFD | 19 | rs10506 | 814,356 |
| CFD | 19 | rs1135146 | 814,388 |
| CFD | 19 | c.*203 | 814,452 |
| | | C3 (Chromosome 19) | |
| C3 | 19 | rs17030 | 6,628,989 |
| C3 | 19 | rs11569565 | 6,629,474 |
| C3 | 19 | rs344555* | 6,630,360 |
| C3 | 19 | rs2277984 | 6,630,511 |
| C3 | 19 | rs7951 | 6,632,991 |
| C3 | 19 | rs11569536 | 6,637,089 |
| C3 | 19 | rs11569511 | 6,641,571 |
| C3 | 19 | rs3745568 | 6,641,613 |
| C3 | 19 | rs11569509* | 6,641,744 |
| C3 | 19 | rs11569508 | 6,641,746 |
| C3 | 19 | rs10414623 | 6,644,163 |
| C3 | 19 | rs389404 | 6,644,387 |
| C3 | 19 | rs423490 | 6,648,406 |
| C3 | 19 | rs366510 | 6,648,829 |
| C3 | 19 | rs428453 | 6,653,157 |
| C3 | 19 | rs432823 | 6,653,246 |
| C3 | 19 | rs11569450 | 6,653,455 |
| C3 | 19 | rs406514 | 6,653,598 |
| C3 | 19 | rs2230205 | 6,660,704 |
| .C3 | 19 | rs2230204 | 6,660,848 |
| C3 | 19 | IVS13 + 64_65* | 6,661,575 |
| C3 | 19 | rs2230203 | 6,661,782 |
| C3 | 19 | rs10411506 | 6,661,948 |
| C3 | 19 | rs11085197* | 6,664,175 |
| C3 | 19 | rs1047286 | 6,664,262 |
| C3 | 19 | rs2230201 | 6,664,291 |
| C3 | 19 | rs2547438 | 6,669,078 |
| C3 | 19 | rs2230199 | 6,669,387 |
| | | C5AR1 (Chromosome19) | |
| C5AR1 | 19 | rs11670330 | 52,505,005 |

TABLE 4
Description of SNPs

| | | | Nucleo- | Amino Acid | Physical | | |
|---|---|---|---|---|---|---|---|
| | | | tide | Change (If | Location | MAF | Frequency |
| SNP | Gene | Region | Change | Any) | (bp) | (Unaffected) | in Affected |
| rs12058824 | C1QA | Promoter | G > A | | 22,835,637 | A = 6.77 | A = 9.40 |
| rs172378 | C1QA | Exon 2 | A > G | Gly92Gly | 22,838,025 | G = 39.10 | G = 38.93 |
| c.-85 | C1QC | Promoter | G > A | | 22,843,019 | A = 2.94 | A = 2.21 |
| rs36049190 | C1QC | Promoter | C > G | | 22,846,286 | G = 4.89 | G = 5.93 |
| rs9434 | C1QC | 3′UTR | C > A | | 22,846,884 | A = 34.19 | A = 35.07 |
| rs291990 | C1QB | Promoter | T > G | | 22,852,242 | G = 44.07 | G = 48.15 |
| rs35906110 | CFH | Promoter | A > G | | 194,887,792 | G = 0.37 | G = 0.00 |
| rs800292 | CFH | Exon 2 | G > A | Val62Ile | 194,908,856 | A = 21.05 | A = 12.31 |
| rs35507625 | CFH | Intron 2 | insTT | | 194,909,593 | TT = 19.55 | TT = 11.74 |
| rs35814900 | CFH | Intron 2 | G > A | | 194,909,603 | A = 2.24 | A = 1.49 |
| rs572515 | CFH | Intron 4 | G > A | | 194,912,884 | A = 39.85 | A = 56.34 |
| rs1061147 | CFH | Exon 7 | C > A | Ala307Ala | 194,920,947 | A = 41.79 | A = 55.97 |
| rs7529589 | CFH | Intron 7 | C > T | | 194,924,902 | T = 41.42 | T = 55.64 |
| rs482934 | CFH | Intron 7 | T > G | | 194,925,120 | G = 41.25 | G = 60.75 |
| rs1061170 | CFH | Exon 9 | T > C | Tyr402His | 194,925,860 | C = 42.54 | C = 55.64 |
| rs12038333 | CFH | Intron 9 | A > G | | 194,939,077 | G = 41.42 | G = 56.34 |
| rs2274700 | CFH | Exon 10 | G > A | Ala473Ala | 194,949,570 | A = 35.82 | A = 19.78 |
| rs203674 | CFH | Intron 10 | T > G | | 194,951,248 | G = 46.24 | G = 62.69 |
| rs3753396 | CFH | Exon 13 | A > G | Gln672Gln | 194,962,365 | G = 16.04 | G = 16.79 |
| rs435628 | CFH | Intron 15 | T > G | | 194,972,509 | G = 0.37 | G = 0.00 |
| rs375046 | CFH | Intron 15 | A > C | | 194,972,549 | C = 47.76 | C = 63.81 |
| rs35292876 | CFH | Exon 17 | C > T | His878His | 194,973,265 | T = 0.75 | T = 1.12 |
| rs515299 | CFH | Exon 17 | G > T | Ile890Ser | 194,973,300 | T = 0.75 | T = 0.00 |
| rs1065489 | CFH | Exon 18 | G > T | Glu936Asp | 194,976,397 | T = 16.42 | T = 17.54 |
| rs16840522 | CFH | Intron 18 | T > C | | 194,977,539 | C = 16.54 | C = 7.58 |
| rs385543 | CFH | Intron 18 | A > G | | 194,977,540 | G = 0.38 | G = 0.00 |
| rs513699 | CFH | Intron 19 | T > C | | 194,979,200 | C = 46.80 | C = 47.18 |
| rs35274867 | CFH | Exon 20 | A > T | Tyr1050Asn | 194,979,219 | T = 1.59 | T = 1.19 |
| rs425757 | CFH | Exon 20 | T > C | Tyr1058His | 194,979,232 | C = 38.31 | C = 42.80 |
| rs410232 | CFH | Exon 20 | G > C | Val1060Lue | 194,979,249 | C = 38.10 | C = 43.20 |
| rs409953 | CFH | Exon 20 | G > A | Thr1097Thr | 194,979,362 | A = 36.90 | A = 40.48 |
| rs3806820 | CFI | Promoter | T > C | | 110,943,016 | C = 5.98 | C = 8.20 |
| c.-380 | CFI | Promoter | C > G | | 110,942,979 | G = 0.00 | G = 0.39 |
| rs3840506 | CFI | Promoter | insA | | 110,942,940 | A = 0.43 | A = 0.82 |
| c.-352 | CFI | Promoter | T > C | | 110,942,928 | C = 1.26 | C = 1.15 |
| c.-13 | CFI | Intron 1 | G > A | | 110,942,489 | A = 4.44 | A = 2.17 |
| rs4382036 | CFI | Intron 1 | A > G | | 110,942,466 | G = 0.00 | G = 0.00 |
| c.191 | CFI | Exon 2 | C > T | Pro64Leu | 110,907,295 | T = 0.00 | T = 0.40 |
| IVS3 + 6 | CFI | Intron 3 | C > T | | 110,905,135 | T = 0.37 | T = 0.77 |
| rs17610314 | CFI | Intron 3 | T > C | | 110,902,402 | C = 24.06 | C = 27.27 |
| IVS3 − 27_25 | CFI | Intron 3 | delATA | | 110,902,324 | — = 0.37 | — = 0.38 |
| rs4382037 | CFI | Intron 5 | A > G | | 110,901,067 | A = 0.00 | A = 0.37 |
| rs5860990 | CFI | Intron 7 | delT | | 110,898,269 | — = 24.81 | — = 21.71 |
| IVS9 + 17 | CFI | Intron 9 | A > G | | 110,890,087 | G = 0.00 | G = 0.38 |
| rs41278047 | CFI | Exon 11 | A > G | Lys441Arg | 110,886,934 | G = 1.14 | G = 1.53 |
| rs9998151 | CFI | Intron 11 | A > G | | 110,886,794 | G = 4.10 | G = 2.80 |
| rs41278049 | CFI | Intron 11 | T > C | | 110,886,789 | C = 1.61 | C = 1.2 |
| IVS12 + 5 | CFI | Intron 12 | G > T | | 110,883,091 | T = 2.22 | T = 2.24 |
| IVS12 + 96 | CFI | Intron 12 | T > A | | 110,882,999 | A = 0.00 | A = 0.37 |
| c.1657 | CFI | Exon 13 | C > T | Pro553Ser | 110,881,593 | T = 0.37 | T = 0.38 |
| rs7746553 | C2 | Intron 2 | C > G | | 32,003,952 | G = 15.91 | G = 11.28 |
| c.386 | C2 | Exon 3 | G > A | Arg129His | 32,004,617 | A = 0.00 | A = 0.38 |
| rs9332739 | C2 | Exon 7 | G > C | Glu318Asp | 32,011,783 | C = 4.10 | C = 3.73 |
| rs9332740 | C2 | Intron 7 | G > T | | 32,011,910 | T = 1.12 | T = 0.37 |
| rs1042663 | C2 | Exon 8 | G > A | Ala341Ala | 32,013,109 | A = 7.46 | A = 7.53 |
| rs550605 | C2 | Intron 9 | T > C | | 32,015,126 | C = 7.04 | C = 7.35 |
| rs2242572 | C2 | Intron 10 | G > A | | 32,018,908 | A = 7.46 | A = 7.46 |
| rs547154 | C2 | Intron 10 | G > T | | 32,018,917 | T = 7.46 | T = 7.46 |
| c.*82 | C2 | 3′UTR | C > T | | 32,021,195 | T = 0.37 | T = 0.74 |
| c.-1476 | CFB | Promoter | T > C | | 32,020,502 | C = 2.22 | C = 1.48 |
| rs4151667 | CFB | Exon 1 | T > A | | 32,022,003 | A = 4.10 | A = 3.73 |
| rs12614 | CFB | Exon 2 | C > T | Arg32Trp | 32,022,158 | T = 9.26 | T = 10.00 |
| rs641153 | CFB | Exon 2 | G > A | Arg32Gln | 32,022,159 | A = 7.46 | A = 7.46 |
| rs1048709 | CFB | Exon 3 | G > A | Arg150Arg | 32,022,914 | A = 15.67 | A = 15.67 |
| rs4151659 | CFB | Exon 13 | A > G | Glu565Lys | 32,026,443 | G = 3.36 | G = 2.61 |
| rs2072633 | CFB | Intron 17 | G > A | | 32,027,557 | A = 39.18 | A = 39.18 |
| rs1683564 | CFD | Promoter | C > A | | 810,214 | A = 32.71 | A = 37.41 |
| c.-322 | CFD | Promoter | G > T | | 810,368 | T = 23.46 | T = 21.11 |
| rs8107095 | CFD | Intron 1 | T > G | | 810,831 | G = 26.47 | G = 25.75 |
| c.522 | CFD | Exon 4 | G > A | Leu174Leu | 812,863 | A = 0.00 | A = 0.37 |
| rs1683561 | CFD | Intron 4 | G > C | | 813,024 | C = 49.62 | C = 51.49 |
| rs10506 | CFD | 3′UTR | C > T | | 814,356 | T = 13.16 | T = 10.82 |
| rs1135146 | CFD | 3′UTR | C > G | | 814,388 | G = 27.94 | G = 28.99 |
| c.*203 | CFD | 3′UTR | delA | | 814,452 | — = 28.20 | — = 29.39 |
| rs2230199 | C3 | Exon 3 | C > G | Arg102Gly | 6,669,387 | G = 21.80 | G = 25.19 |
| rs2547438 | C3 | Intron 3 | A > C | | 6,669,078 | C = 18.80 | C = 16.92 |
| rs2230201 | C3 | Exon 9 | G > A | Arg304Arg | 6,664,291 | A =13.97 | A = 15.07 |
| rs1047286 | C3 | Exon 9 | C > T | Pro314Leu | 6,664,262 | T = 19.85 | T = 21.69 |
| rs11085197 | C3 | Intron 9 | C > G | | 6,664,175 | G = 19.17 | G = 22.22 |
| rs10411506 | C3 | Intron 12 | C > T | | 6,661,948 | T = 12.41 | T = 11.19 |
| rs2230203 | C3 | Exon 13 | C > A | Pro518Leu | 6,661,782 | A = 17.67 | A = 21.97 |
| IVS13 + 64-65 | C3 | Intron 13 | delTC | | 6,661,575 | — = 31.20 | — = 30.71 |
| rs2230204 | C3 | Exon 14 | G/A/C/T | Val564Val | 6,660,848 | A = 27.57 | A = 26.52 |
| rs2230205 | C3 | Exon 14 | G > A | Thr612Thr | 6,660,704 | A = 12.22 | A = 12.12 |
| rs406514 | C3 | Intron 17 | T > C | | 6,653,598 | C = 20.22 | C = 27.78 |
| rs11569450 | C3 | Intron 18 | C > G | | 6,653,455 | G = 11.28 | G = 10.45 |
| rs432823 | C3 | Intron 18 | G > A | | 6,653,246 | A = 31.25 | A = 34.96 |
| rs428453 | C3 | Exon 19 | C > G | Val807Val | 6,653,157 | G = 30.83 | G = 34.81 |
| rs366510 | C3 | Intron 19 | A > C | | 6,648,829 | C = 31.25 | C = 35.19 |
| rs423490 | C3 | Exon 21 | C > T | Ala915Ala | 6,648,406 | T = 20.74 | T = 28.46 |
| rs389404 | C3 | Intron 25 | C > T | | 6,644,387 | T = 10.90 | T = 9.47 |
| rs10414623 | C3 | Intron 25 | C > T | | 6,644,163 | T = 7.14 | T = 6.77 |
| rs11569508 | C3 | Intron 26 | T > C | | 6,641,746 | C = 10.66 | C = 9.56 |
| rs11569509 | C3 | Intron 26 | T > C | | 6,641,744 | C = 9.56 | C = 9.56 |
| rs3745568 | C3 | Intron 27 | A > C | | 6,641,613 | C = 11.28 | C = 9.26 |
| rs11569511 | C3 | Intron 27 | G > A | | 6,641,571 | A = 10.66 | A = 9.09 |
| rs11569536 | C3 | Intron 29 | C > T | | 6,637,089 | T = 7.89 | T = 7.84 |
| rs7951 | C3 | Exon 35 | C > T | Ala1437Ala | 6,632,991 | T = 6.39 | T = 4.81 |
| rs2277984 | C3 | Intron 36 | A > G | | 6,630,511 | G = 44.49 | G = 46.18 |
| rs344555 | C3 | Intron 37 | G > A | | 6,630,360 | A = 20.59 | A = 23.53 |
| rs11569565 | C3 | Intron 38 | C > T | | 6,629,474 | T = 8.09 | T = 7.72 |
| rs17030 | C3 | Exon 41 | T > C | Pro1632Pro | 6,628,989 | C = 44.49 | C = 46.67 |
| rs11670330 | C5AR1 | Intron 1 | G > A | | 52,505,005 | A = 27.44 | A = 28.89 |

TABLE 5a
Results of Single Factor Conditional Logistic Regression Analysis for the CFH Gene

| | | CLR Odds Ratio | CLR Mode | | Disease |
|---|---|---|---|---|---|
| | Minor | (95% C.I.) for Mode | of | CLR p | Effect of |
| SNP | Allele | of Inheritance | inheritance | value | Minor Allele |
| rs800292 | A | 0.249 (0.111-0.559) | Add | 0.0008 | Protective |
| rs35507625 | TT | 0.292 (0.126-0.677) | Dom | 0.0041 | Protective |
| rs5814900 | A | 0.333 (0.035-3.205) | Add/Dom | 0.3414 | Protective |
| rs572515 | A | 4.297 (2.282-8.091) | Add | 6.32E−06 | Increased Risk |
| rs1061147 | A | 2.867 (1.680-4.893) | Add | 0.0001 | Increased Risk |
| rs7529589 | T | 7.500 (2.642-21.289) | Rec | 0.0002 | Increased Risk |
| rs482934 | G | 3.632 (2.033-6.488) | Add | 1.33E−05 | Increased Risk |
| rs1061170 | C | 5.800 (2.245-14.983) | Rec | 0.0003 | Increased Risk |
| rs12038333 | G | 7.500 (2.642-21.289) | Rec | 0.0002 | Increased Risk |
| rs2274700 | A | 0.249 (0.133-0.468) | Add | 1.54E−05 | Protective |
| rs203674 | G | 3.656 (2.039-6.557) | Add | 1.36E−05 | Increased Risk |
| rs3753396 | G | 1.100 (0.600-2.016) | Add | 0.7580 | Increased Risk |
| rs375046 | C | 3.077 (1.811-5.226) | Add | 3.21E−05 | Increased Risk |
| rs1065489 | T | 1.158 (0.626-2.142) | Add | 0.6399 | Increased Risk |
| rs16840522 | C | 0.329 (0.160-0.676) | Add | 0.0025 | Protective |
| rs513699 | C | 1.333 (0.463-3.843) | Add/Dom | 0.5943 | Increased Risk |
| rs425757 | C | 1.583 (0.769-3.262) | Add | 0.2127 | Increased Risk |
| rs2410232 | C | 2.000 (0.970-4.124) | Add | 0.0605 | Increased Risk |
| rs409953 | A | 1.750 (0.861-3.557) | Add/Dom | 0.122 | Increased Risk |

TABLE 5b
Results of Single Factor Conditional Logistic Regression Analysis for the C3 Gene

| | | CLR Odds Ratio | CLR Mode | | Disease |
|---|---|---|---|---|---|
| | | (95% C.I.) for Mode | of | CLR p | Effect of |
| SNP | Allele | of Inheritance | Inheritance | value | Minor Allele |
| rs2230199 | G | 1.434 (0.814-2.525) | Add | 0.2122 | Increased Risk |
| rs2547438 | C | 0.682 (0.354-1.314) | Dom | 0.2527 | Protective |
| rs2230201 | A | 1.167 (0.621-2.192) | Add | 0.6315 | Increased Risk |
| rs1047286 | T | 1.302 (0.724-2.341) | Add | 0.3788 | Increased Risk |
| rs11085197 | G | 1.538 (0.765-3.093) | Dom | 0.1326 | Increased Risk |
| rs10411506 | T | 0.733 (0.337-1.597) | Dom | 0.4346 | Protective |
| rs2230203 | A | 9.000 (1.140-71.036) | Rec | 0.0371 | Increased Risk |
| IVS13 + 64-65 | — | 1.176 (0.616-2.246) | Dom | 0.6403 | Increased Risk |
| rs2230204 | A | 0.778 (0.290-2.088) | Rec | 0.4692 | Protective |
| rs2230205 | A | 1.500 (0.251-8.977) | Rec | 0.6569 | Increased Risk |
| rs406514 | C | 1.857 (1.094-3.152) | Add | 0.0219 | Increased Risk |
| rs11569450 | G | 0.667 (0.111-3.990) | Rec | 0.6569 | Protective |
| rs432823 | A | 1.500 (0.674-3.339) | Rec | 0.3206 | Increased Risk |
| rs428453 | G | 1.500 (0.674-3.339) | Rec | 0.2436 | Increased Risk |
| rs366510 | C | 1.272 (0.824-1.964) | Add | 0.3309 | Increased Risk |
| rs423490 | T | 1.904 (1.123-3.229) | Add | 0.0168 | Increased Risk |
| rs389404 | T | 0.600 (0.263-1.371) | Dom | 0.1673 | Protective |
| rs10414623 | T | 1.125 (0.434-2.915) | Add | 0.8275 | Increased Risk |
| rs11569508 | C | 0.563 (0.249-1.273) | Dom | 0.1673 | Protective |
| rs11569509 | C | 0.563 (0.249-1.273) | Dom | 0.1673 | Protective |
| rs3745568 | C | 0.620 (0.276-1.394) | Add | 0.3412 | Protective |
| rs11569511 | A | 0.563 (0.249-1.273) | Dom | 0.1673 | Protective |
| rs11569536 | T | 0.875 (0.427-1.793) | Dom | 0.8575 | Protective |
| rs7951 | T | 0.700 (0.266-1.839) | Add/Dom | 0.3499 | Protective |
| rs2277984 | G | 1.700 (0.778-3.713) | Rec | 0.4665 | Increased Risk |
| rs344555 | A | 3.333 (0.917-12.112) | Rec | 0.0674 | Increased Risk |
| rs11569565 | T | 0.800 (0.215-2.979) | Add | 0.7394 | Protective |
| rs17030 | C | 1.700 (0.778-3.713) | Rec | 0.2606 | Increased Risk |

TABLE 5c
Results of Single Factor Conditional Logistic
Regression Analysis for the CFD Gene

| | | CLR Odds Ratio | CLR Mode | | Disease |
|---|---|---|---|---|---|
| | | (95% C.I.) for Mode | of | CLR p | Effect of |
| SNP | Allele | of Inheritance | Inheritance | value | Minor Allele |
| rs1683564 | A | 2.364 (1.168-4.783) | Dom | 0.0168 | Increased Risk |
| c.-322 | T | 0.200 (0.023-1.712) | Rec | 0.1418 | Protective |
| rs8107095 | G | 0.870 (0.478-1.584) | Add | 0.6483 | Protective |
| rs1683561 | G | 0.778 (0.387-1.564) | Rec | 0.3859 | Protective |
| rs10506 | T | 0.538 (0.215-1.350) | Add/Dom | 0.1867 | Protective |
| rs1135146 | G | 1.087 (0.617-1.915) | Add | 0.7731 | Increased Risk |
| c.*203 | — | 0.933 (0.451-1.934) | Dom | 0.7818 | Protective |

TABLE 5d
Results of Single Factor Conditional Logistic Regression Analysis for
Complement Factor Genes C1QA, C1QC, C1QB, CFI, C2, CFB, and C5AR1

| | | | | | Disease |
|---|---|---|---|---|---|
| | | | | | Effect of |
| SNP | Gene | Odds Ratio (95% C.I.) | p value | Model | Minor Allele |
| rs12058824 | C1QA | 3.000(0.968-9.302) | 0.0571 | Add | Increased risk |
| rs172378 | C1QA | 1.100(0.467-2.590) | 0.8275 | Dom | Increased risk |
| c.-85 | C1QC | 0.333(0.035-3.205) | 0.3414 | Add/Dom | Protective |
| rs36049190 | C1QC | 1.680(0.596-4.735) | 0.3262 | Add | Increased risk |
| rs9434 | C1QC | 1.100(0.467-2.590) | 0.8275 | Rec | Increased risk |
| rs291990 | C1QB | 2.000(0.751-5.328) | 0.1657 | Dom | Increased risk |
| rs3806820 | CFI | 1.667(0.606-4.586) | 0.3226 | Add/Dom | Increased risk |
| c.-380 | CFI | 1.473E+6(0-Inf.) | 0.9907 | Add/Dom | Increased risk |
| rs3840506 | CFI | 4.004E+6(0-Inf.) | 0.9914 | Add/Dom | Increased risk |
| c.-352 | CFI | 0.000(0-Inf) | 0.9907 | Add/Dom | n/a |
| c.-13 | CFI | 1.600(0.523-4.891) | 0.4097 | Add/Dom | Increased risk |
| rs4382036 | CFI | 0.000(0-Inf.) | 0.9914 | Add/Dom | n/a |
| c.191 | CFI | 1.473E+6(0-Inf.) | 0.9907 | Add/Dom | Increased risk |
| IVS3 + 6 | CFI | 0.000(0-Inf.) | 0.9907 | Add/Dom | n/a |
| rs17610314 | CFI | 0.640(0.342-1.199) | 0.1633 | Dom | Protective |
| rs4382037 | CFI | 1.473E+6(0-Inf.) | 0.9907 | Add/Dom | Increased risk |
| rs5860990 | CFI | 0.739(0.395-1.383) | 0.3446 | Dom | Protective |
| IVS9 + 17 | CFI | 1.473E+6(0-Inf.) | 0.9907 | Add/Dom | Increased risk |
| rs41278047 | CFI | 2.000(0.181-22.053) | 0.5715 | Add/Dom | Increased risk |
| rs9998151 | CFI | 0.667(0.188-2.362) | 0.5299 | Add/Dom | Protective |
| rs41278049 | CFI | 0.000(0-Inf.) | 0.9907 | Add/Dom | n/a |
| IVS12 + 5 | CFI | 1.000(0.202-4.955) | 1 | Add/Dom | n/a |
| c.1657 | CFI | 1.473E+6(0-Inf.) | 0.9907 | Add/Dom | Increased risk |
| rs7746553 | C2 | 0.500(0.225-1.113) | 0.0896 | Dom | Protective |
| c.386 | C2 | 1.473E+6(0-Inf.) | 0.9907 | Add/Dom | Increased risk |
| rs9332739 | C2 | 0.800(0.215-2.979) | 0.7394 | Add/Dom | Protective |
| rs9332740 | C2 | 0.000(0-Inf) | 0.9914 | Add/Dom | n/a |
| rs1042663 | C2 | 1.000(0.434-2.307) | 1 | Add/Dom | n/a |
| rs550605 | C2 | 1.100(0.467-2.590) | 0.8275 | Add/Dom | Protective |
| rs2242572 | C2 | 1.000(0.434-2.307) | 1 | Add/Dom | n/a |
| rs547154 | C2 | 1.000(0.434-2.307) | 1 | Add/Dom | n/a |
| c.*82 | C2 | 1.473E+6(0-Inf.) | 0.9907 | Add/Dom | Increased risk |
| c.-1476 | CFB | 0.500(0.092-2.739) | 0.4236 | Add/Dom | Protective |
| rs4151667 | CFB | 0.800(0.215-2.979) | 0.7394 | Add/Dom | Protective |
| rs12614 | CFB | 3.000(0.312-28.841) | 0.3414 | Rec | Increased risk |
| rs641153 | CFB | 1.000(0.434-2.307) | 1 | Add/Dom | n/a |
| rs1048709 | CFB | 1.062(0.537-2.103) | 0.8619 | Add | Increased risk |
| rs4151659 | CFB | 0.333(0.035-3.205) | 0.3414 | Add/Dom | Protective |
| rs2072633 | CFB | 0.943(0.586-1.517) | 0.8085 | Add | Protective |
| rs11670330 | C5AR1 | 1.235(0.652-2.341) | 0.5172 | Dom | Increased risk |

TABLE 6a
Single-Marker Analysis Results from the FBAT Program;
the genetic models are the same as for CLR for the
CFH Gene, without Bonferroni Correction

| | | Number of | | | |
|---|---|---|---|---|---|
| | | Informative | Variance | Z | Pairwise |
| SNP | Allele | Families | (S) | Value | p value |
| rs800292 | A | 36 | 10.5 | −3.703 | 0.0002 |
| rs35507625 | TT | 35 | 10.25 | −2.967 | 0.0023 |
| rs572515 | A | 61 | 19.75 | 5.063 | 4.13E−07 |
| rs1061147 | A | 63 | 21 | 4.146 | 3.40E−05 |
| rs7529589 | T | 64 | 22 | 4.051 | 8.24E−06 |
| rs482934 | G | 63 | 21.75 | 4.825 | 1.40E−06 |
| rs1061170 | C | 62 | 20.75 | 3.842 | 3.90E−05 |
| rs12038333 | G | 63 | 21 | 4.364 | 8.24E−06 |
| rs2274700 | A | 58 | 19.75 | −4.838 | 1.31E−06 |
| rs203674 | G | 63 | 21 | 4.801 | 1.58E−06 |
| rs3753396 | G | 36 | 10.5 | 0.309 | 0.7576 |
| rs375046 | C | 67 | 22.75 | 4.508 | 6.56E−06 |
| rs1065489 | T | 35 | 10.25 | 0.469 | 0.63941 |
| rs16840522 | C | 37 | 11.5 | −3.244 | 0.0012 |
| rs513699 | C | 14 | 3.5 | 0.535 | 0.5930 |
| rs425757 | C | 31 | 7.75 | 1.257 | 0.2067 |
| rs2410232 | C | 33 | 8.25 | 1.915 | 0.0555 |
| rs409953 | A | 33 | 8.25 | 1.567 | 0.1172 |

TABLE 6b
Single-Marker Analysis Results from the FBAT Program
the genetic models are the same as for CLR for
the C3 Gene, without Bonferroni correction

| | | Number of | | | |
|---|---|---|---|---|---|
| | | Informative | Variance | Z | Pairwise |
| SNP | Allele | Families | (S) | Value | p value |
| rs2230199 | G | 39 | 12.75 | 1.26 | 0.2076 |
| rs2547438 | C | 41 | 11.75 | −0.729 | 0.2498 |
| rs2230201 | A | 33 | 9.75 | 0.48 | 0.631 |
| rs1047286 | T | 37 | 11.5 | 0.885 | 0.3763 |
| rs11085197 | G | 37 | 11.5 | 1.18 | 0.223 |
| rs10411506 | T | 29 | 8.75 | −0.507 | 0.4328 |
| rs2230203 | A | 37 | 13.75 | 1.483 | 0.0114 |
| IVS13 + 64-65 | — | 47 | 21.5 | 0 | 0.5316 |
| rs2230204 | A | 52 | 18.25 | −0.351 | 0.6171 |
| rs2230205 | A | 36 | 10.5 | 0 | 0.6547 |
| rs406514 | C | 50 | 16.25 | 2.357 | 0.0184 |
| rs11569450 | G | 31 | 8.5 | −0.343 | 0.6547 |
| rs432823 | A | 60 | 21 | 0.873 | 0.3173 |
| rs428453 | G | 60 | 21 | 0.873 | 0.3173 |
| rs366510 | C | 60 | 21 | 1.091 | 0.2752 |
| rs423490 | T | 51 | 16.5 | 2.462 | 0.0138 |
| rs389404 | T | 26 | 7.25 | −0.557 | 0.2207 |
| rs10414623 | T | 20 | 5 | 0 | 0.8084 |
| rs11569508 | C | 27 | 7.5 | −0.73 | 0.1615 |
| rs11569509 | C | 27 | 7.5 | −0.73 | 0.1615 |
| rs3745568 | C | 20 | 6.5 | −1.177 | 0.2393 |
| rs11569511 | A | 27 | 7.5 | −0.73 | 0.1615 |
| rs11569536 | T | 30 | 8.25 | −0.174 | 0.715 |
| rs7951 | T | 17 | 4.25 | −0.728 | 0.4669 |
| rs2277984 | G | 56 | 19.25 | 0.798 | 0.1779 |
| rs344555 | A | 38 | 11.75 | 1.313 | 0.0522 |
| rs11569565 | T | 9 | 2.25 | −0.333 | 0.7389 |
| rs17030 | C | 56 | 19.25 | 0.798 | 0.1779 |

TABLE 6c
Single-Marker Analysis Results from the FBAT Program
the genetic models are the same as for CLR for
the CFD Gene, without Bonferroni Correction

| | | Number of | | | |
|---|---|---|---|---|---|
| | | Informative | Variance | Z | Pairwise |
| SNP | Allele | Families | (S) | Value | p value |
| rs1683564 | A | 55 | 16.75 | 1.588 | 0.0137 |
| c.-322 | T | 35 | 10.25 | −0.781 | 0.1025 |
| rs8107095 | G | 40 | 10.75 | −0.457 | 0.6473 |
| rs1683561 | G | 54 | 18 | −0.471 | 0.4795 |
| rs10506 | T | 20 | 5 | −1.342 | 0.1797 |
| rs1135146 | G | 42 | 12 | 0.289 | 0.7728 |
| c.*203 | — | 39 | 11.25 | −0.149 | 0.8527 |

TABLE 6d
Single-Marker Analysis Results from the FBAT Program
for the CFH Gene, after Bonferroni correction

| | | Number of | | | Family |
|---|---|---|---|---|---|
| | | Informative | Variance | Z | wise |
| SNP | Allele | Families | (S) | Value | p value |
| rs800292 | A | 36 | 10.5 | −3.703 | 0.003 |
| rs35507625 | TT | 35 | 10.25 | −2.967 | 0.039 |
| rs5814900 | A | 4 | 1 | −1 | 0.31731 |
| rs572515 | A | 61 | 19.75 | 5.063 | 6.2E−06 |
| rs1061147 | A | 63 | 21 | 4.146 | 0.00051 |
| rs7529589 | T | 64 | 22 | 4.051 | 1.24E−04 |
| rs482934 | G | 63 | 21.75 | −4.825 | 2.10E−05 |
| rs1061170 | C | 62 | 20.75 | 3.842 | 5.85E−04 |
| rs12038333 | G | 63 | 21 | 4.364 | 1.24E−04 |
| rs2274700 | A | 58 | 19.75 | −4.838 | 1.97E−05 |
| rs203674 | G | 63 | 21 | 4.801 | 2.37E−05 |
| rs3753396 | G | 36 | 10.5 | 0.309 | 0.61209 |
| rs375046 | C | 67 | 22.75 | 4.508 | 9.84E−05 |
| rs1065489 | T | 35 | 10.25 | 0.469 | 0.49272 |
| rs16840522 | C | 37 | 11.5 | −3.244 | 0.0177 |
| rs513699 | C | 14 | 3.5 | 0.535 | 0.59298 |
| rs425757 | C | 31 | 7.75 | 1.257 | 0.14413 |
| rs2410232 | C | 33 | 8.25 | 1.915 | 0.8327 |
| rs409953 | A | 33 | 8.25 | 1.567 | 0.11719 |

TABLE 8
Genotype and Allele Frequencies of three SNPs of the CFH gene

| | | Affected Siblings | | | |
|---|---|---|---|---|---|
| | | (Index cases) | | Unaffected Siblings | |
| | | Frequency | | Frequency | |
| SNP | | (%) | No. | (%) | No. |
| | Genotype | | | | |
| rs35507625 | — | 79.55% | 105 | 65.41% | 87 |
| | TT, — | 17.42% | 23 | 30.08% | 40 |
| | TT, TT | 3.03% | 4 | 4.51% | 6 |
| | Total | | 132 | | 133 |
| | Allele | | | | |
| | — | 88.26% | 233 | 80.45% | 214 |
| | T | 11.74% | 31 | 19.55% | 52 |
| | | | 264 | | 266 |
| | Genotype | | | | |
| rs482934 | TT | 20.56% | 22 | 35.83% | 43 |
| | GT | 37.38% | 40 | 45.83% | 55 |
| | GG | 42.06% | 45 | 18.33% | 22 |
| | Total | | 107 | | 120 |
| | Allele | | | | |
| | T | 39.25% | 84 | 58.75% | 141 |
| | G | 60.75% | 130 | 41.25% | 99 |
| | | | 214 | | 240 |
| | Genotype | | | | |
| rs375046 | CC | 44.03% | 59 | 23.88% | 32 |
| | AC | 39.55% | 53 | 47.76% | 64 |
| | AA | 16.42% | 22 | 28.36% | 38 |
| | Total | | 134 | | 134 |
| | Allele | | | | |
| | C | 63.81% | 171 | 47.76% | 128 |
| | A | 36.19% | 97 | 52.24% | 140 |
| | | | 268 | | 268 |

TABLE 9
Multiple Conditional Logistic Regression Analysis Showing Significant
Single-Nucleotide Polymorphisms for the CFH gene

| | | | |
|---|---|---|---|
| | Refer- | Odds Ratio (95% | |
| Risk Factor | ent | C.I.) | p value |

CFH rs800292 (G > A): “A” is minor allele

| | | | |
|---|---|---|---|
| rs800292 AG | GG | 0.233 (0.078-0.702) | 0.0096 |
| rs800292 AA | GG | 0.129 (0.010-1.596) | 0.1106 |
| Smoking ≧10 | <10 | 2.817 (1.468-5.408) | 0.0018 |
| pack-years | pack- | | |
| | years | | |
| rs1049331 TC | CC | 5.665 (2.126-15.091) | 0.0005 |
| rs1049331 TT | CC | 48.845 (9.318-256.034) | 4.21E−06 |

CFH rs35507625 (insTT): “TT” is minor allele

| | | | |
|---|---|---|---|
| rs35507625 TT, — | — | 0.280 (0.099-0.781) | 0.0163 |
| rs35507625 TT, TT | — | 0.329 (0.032-3.404) | 0.3513 |
| Smoking ≧10 | <10 | 3.011 (1.550-5.849) | 0.0011 |
| pack-years | pack- | | |
| | years | | |
| rs1049331 TC | CC | 5.773 (2.140-15.575) | 0.0005 |
| rs1049331 TT | CC | 54.392 (10.366-285.407) | 2.30E−06 |

CFH rs572515 (G > A): “A” is minor allele

| | | | |
|---|---|---|---|
| rs572515 AG | GG | 3.528 (1.241-10.026) | 0.0180 |
| rs572515 AA | GG | 71.095 (9.826-514.398) | 2.41E−05 |
| Smoking ≧10 | <10 | 4.380 (1.955-9.812) | 0.0003 |
| pack-years | pack- | | |
| | years | | |
| rs1049331 TC | CC | 5.944 (2.039-17.330) | 0.0011 |
| rs1049331 TT | CC | 106.847 (14.694-776.913) | 3.93E−06 |

CFH rs1061147 (C > A): “A” is minor allele

| | | | |
|---|---|---|---|
| rs1061147 AC | CC | 2.051 (0.824-5.106) | 0.1228 |
| rs1061147 AA | CC | 20.688 (4.530-94.478) | 9.25E−05 |
| Smoking ≧10 | <10 | 3.884 (1.854-8.136) | 0.0003 |
| pack-years | pack- | | |
| | years | | |
| rs1049331 TC | CC | 6.288 (2.168-18.237) | 0.0007 |
| rs1049331 TT | CC | 109.203 (15.019-794.013) | 3.54E−06 |

CFH rs7529589 (C > T): “T” is minor allele

| | | | |
|---|---|---|---|
| rs7529589 TC | CC | 1.828 (0.748-4.465) | 0.1855 |
| rs7529589 TT | CC | 24.077 (4.826-120.121) | 0.0001 |
| Smoking ≧10 | <10 | 3.920 (1.842-8.340) | 0.0004 |
| pack-years | pack- | | |
| | years | | |
| rs1049331 TC | CC | 5.293 (1.868-14.997) | 0.0017 |
| rs1049331 TT | CC | 98.786 (13.697-712.472) | 5.21E−06 |

CFH rs482934 (T > G): “G” is minor allele

| | | | |
|---|---|---|---|
| rs482934 GT | TT | 2.151 (0.752-6.156) | 0.1533 |
| rs482934 GG | TT | 17.716 (3.978-78.900) | 0.0002 |
| Smoking ≧10 | <10 | 3.372 (1.600-7.107) | 0.0014 |
| pack-years | pack- | | |
| | years | | |
| rs1049331 TC | CC | 5.780 (1.976-16.909) | 0.0014 |
| rs1049331 TT | CC | 79.369 (10.864-579.83)1 | 1.62E−05 |

CFH rs1061170 (T > C): “C” is minor allele

| | | | |
|---|---|---|---|
| rs1061170 CT | TT | 1.670 (0.664-4.198) | 0.2754 |
| rs1061170 CC | TT | 19.291 (4.280-86.941) | 0.0001 |
| Smoking ≧10 | <10 | 4.090 (1.931-8.664) | 0.0002 |
| pack-years | pack- | | |
| | years | | |
| rs1049331 TC | CC | 5.971 (2.098-16.988) | 0.0008 |
| rs1049331 TT | CC | 105.524 (14.642-760.501) | 3.78E−06 |

CFH rs12038333 (A > G): “G” is minor allele

| | | | |
|---|---|---|---|
| rs12038333 GA | AA | 2.114 (0.831-5.380) | 0.1161 |
| rs12038333 GG | AA | 28.500 (5.446-149.150) | 7.28E−05 |
| Smoking ≧10 | <10 | 4.094 (1.915-8.749) | 0.0003 |
| pack-years | pack- | | |
| | years | | |
| rs1049331 TC | CC | 5.392 (1.886-15.419) | 0.0017 |
| rs1049331 TT | CC | 106.187 (14.150-796.847) | 5.71E−06 |

CFH rs2274700 (G > A): “A” is minor allele

| | | | |
|---|---|---|---|
| rs2274700 AG | GG | 0.136 (0.048-0.381) | 0.0002 |
| rs2274700 AA | GG | 0.085 (0.016-0.436) | 0.0031 |
| Smoking ≧10 | <10 | 3.895 (1.853-8.187) | 0.0003 |
| pack-years | pack- | | |
| | years | | |
| rs1049331 TC | CC | 5.660 (2.034-15.749) | 0.0009 |
| rs1049331 TT | CC | 63.813 (10.604-384.004) | 5.66E−06 |

CFH rs203674 (T > G): “G” is minor allele

| | | | |
|---|---|---|---|
| rs203674 GT | TT | 3.032 (0.953-9.643) | 0.0602 |
| rs203674 GG | TT | 31.945 (5.708-178.768) | 8.06E−05 |
| Smoking ≧10 | <10 | 4.306 (1.945-9.536) | 0.0003 |
| pack-years | pack- | | |
| | years | | |
| rs1049331 TC | CC | 4.792 (1.704-13.475) | 0.0030 |
| rs1049331 TT | CC | 157.624 (17.090-1453.757) | 8.04E−06 |

CFH rs37S046 (A > C): “C” is minor allele

| | | | |
|---|---|---|---|
| rs375046 CA | AA | 2.455 (0.812-7.420) | 0.1116 |
| rs375046 CC | AA | 20.491 (4.300-97.648) | 0.0002 |
| Smoking ≧10 | <10 | 4.579 (2.059-10.183) | 0.0002 |
| pack-years | pack- | | |
| | years | | |
| rs1049331 TC | CC | 5.492 (1.905-15.836) | 0.0016 |
| rs1049331 TT | CC | 156.593 (17.751-1381.364) | 5.38E−06 |

CFH rs16840522 (T > C): “C” is minor allele

| | | | |
|---|---|---|---|
| rs16840522 CT | TT | 0.364 (0.137-0.967) | 0.0427 |
| rs16840522 CC | TT | n/a | 0.9910 |
| Smoking ≧10 | <10 | 3.370 (1.692-6.712) | 0.0005 |
| pack-years | pack- | | |
| | years | | |
| rs1049331 TC | CC | 5.385 (1.984-14.614) | 0.0009 |
| rs1049331 TT | CC | 47.911 (9.447-242.997) | 3.00E−06 |

1. A method of determining an individual's susceptibility to age-related macular degeneration (AMD), comprising testing a biological sample obtained from the individual for the presence or absence of an allelic variant of the Complement Factor H(CFH) gene, wherein the allelic variant is a guanine at polymorphic site rs482934, and wherein the presence of the allelic variant indicates that the individual has a significant risk for developing AMD.

2. A method of determining an individual's susceptibility to age-related macular degeneration (AMD), comprising testing a biological sample obtained from the individual for the presence or absence of an allelic variant of the Complement Factor H(CFH) gene, wherein the allelic variant is a cytosine at polymorphic position rs375046, and wherein the presence of the allelic variant indicates that the individual has a significant risk for developing AMD.

3. A method of determining an individual's susceptibility to age-related macular degeneration (AMD), comprising testing a biological sample obtained from the individual for the presence or absence of an allelic variant of the Complement Factor H(CFH) gene, wherein the allelic variant is a cytosine at polymorphic site rs16840522 and wherein the presence of the allelic variant indicates that the individual has a significantly reduced susceptibility to AMD.

4. A method of determining an individual's susceptibility to age-related macular degeneration (AMD), comprising testing a biological sample obtained from the individual for the presence or absence of an allelic variant of the Complement Factor D (CFD) gene, wherein the allelic variant is a guanine at polymorphic site rs1683564, and wherein the presence of the allelic variant indicates that the individual has a reduced susceptibility to AMD.

5. The method of claim 1, wherein the individual is homozygous for the allelic variant.

6. The method of claim 1, wherein the individual is heterozygous for the allelic variant.

7. The method of claim 1 wherein the testing step includes:
(i) combining the biological sample with one or more probes, wherein each of said one or more probes is characterized by its ability to bind with specificity to an allelic variant in the sample, and
(ii) observing the presence or absence of binding between the probe and a macromolecule within the biological sample.

8. The method of claim 7, wherein the probe is an oligonucleotide or an oligonucleotide derivative.

9. The method of claim 8, wherein the probe is an oligonucleotide capable of priming polynucleotide synthesis in a polymerase chain reaction.

10. The method of claim 7, wherein the probe is an antibody or an antibody derivative.

11. A method of determining an individual's susceptibility to age-related macular degeneration (AMD), comprising testing a biological sample obtained from the individual for the presence or absence of a polymorphic haplotype, wherein the haplotype comprises a guanine at polymorphic site rs800292 and a common allele of no insertion at polymorphic site rs35507625, and wherein the presence of the allelic variant indicates that the individual has an increased susceptibility to AMD.

12. A method of determining an individual's susceptibility to age-related macular degeneration (AMD), comprising testing a biological sample obtained from the individual for the presence or absence of a polymorphic haplotype, wherein the haplotype comprises an adenine at polymorphic site rs800292 and a thymine-thymine double nucleotide insertion at polymorphic site rs35507625, and wherein the presence of the haplotype indicates that the individual has a reduced susceptibility to AMD.

13. A method of determining an individual's susceptibility to age-related macular degeneration (AMD), comprising testing a biological sample obtained from the individual for the presence or absence of a polymorphic haplotype, wherein the haplotype comprises a nucleic acid segment comprising an adenine at polymorphic site rs572515, and wherein the presence of the haplotype indicates that the individual has an increased susceptibility to developing AMD.

14. A method of determining an individual's′ susceptibility to age-related macular degeneration (AMD), comprising testing a biological sample obtained from the individual for the presence or absence of a polymorphic haplotype, wherein the haplotype comprises an adenine at polymorphic site rs572515, an adenine at polymorphic site rs1061147, a thymine at polymorphic site rs7529589, a guanine at polymorphic site rs482934, a cytosine at polymorphic site rs1061170, a guanine at polymorphic site rs12038333, a guanine at polymorphic sited rs2274700, a guanine at polymorphic site rs203674, an adenine at polymorphic site rs3753396, a cytosine at polymorphic site rs375046, and a guanine at polymorphic site rs1065489, and wherein the presence of the haplotype indicates that the individual has an increased susceptibility to AMD.

15. A method of determining an individual's susceptibility to age-related macular degeneration (AMD), comprising testing a biological sample obtained from the individual for the presence or absence of a polymorphic haplotype, wherein the haplotype comprises a guanine at polymorphic site rs572515, a cytosine at polymorphic site rs1061147, a cytosine at polymorphic site rs7529589, a thymine at polymorphic site rs482934, a thymine at polymorphic site rs1061170, an adenine at polymorphic site rs12038333, an adenine at polymorphic sited rs2274700, a thymine at polymorphic site rs203674, an adenine at polymorphic site rs3753396, an adenine at polymorphic site rs375046, and a guanine at polymorphic site rs1065489, and wherein the presence of the haplotype indicates that the individual has a reduced susceptibility to AMD.

16. A method of determining an individual's susceptibility to age-related macular degeneration (AMD), comprising, testing a biological sample obtained from the individual for the presence or absence of a polymorphic haplotype, wherein the haplotype comprises a guanine at polymorphic site rs572515, a cytosine at polymorphic site rs1061147, a cytosine at polymorphic site rs7529589, a thymine at polymorphic site rs482934, a thymine at polymorphic site rs1061170, an adenine at polymorphic site rs12038333, a guanine at polymorphic sited rs2274700, a thymine at polymorphic site rs203674, a guanine at polymorphic site rs3753396, an adenine at polymorphic site rs375046, and a guanine at polymorphic site rs1065489, and wherein the presence of the haplotype indicates that the individual has &reduced susceptibility to AMD.

17. A method of determining an individual's susceptibility to age-related macular degeneration (AMD), comprising testing a biological sample obtained from the individual for the presence or absence of a polymorphic haplotype, wherein the haplotype comprises a thymine at polymorphic site rs16840522, a cytosine at polymorphic site rs513699, a cytosine at polymorphic site rs425757, and a cytosine at polymorphic site rs410232, and wherein the presence of the haplotype indicates that the individual has an increased susceptibility to developing AMD.

18. A method of determining an individual's susceptibility to age-related macular degeneration (AMD), comprising testing a biological sample obtained from the individual for the presence or absence of a polymorphic haplotype, wherein the haplotype comprises a cytosine at polymorphic site rs16840522, a thymine at polymorphic site rs513699, a thymine at polymorphic site rs425757, and a guanine at polymorphic site rs410232, and wherein the presence of the haplotype indicates that the individual has a decreased susceptibility to AMD.

19. A method of determining an individual's susceptibility to age-related macular degeneration (AMD), comprising testing a biological sample obtained from the individual for the presence or absence of a polymorphic haplotype, wherein the haplotype comprises a cytosine at polymorphic site rs7951, a guanine at polymorphic site rs2277984, an adenine at polymorphic site rs344555, a cytosine at polymorphic site rs 11569565, and a, cytosine at polymorphic site rs 17030.

20. The method of claim 11, wherein the testing step includes:
(i) combining said biological sample with a plurality of probes, wherein said plurality of probes includes at least one probe characterized by its ability to bind with specificity to each polymorphic site in the sample, and
(ii) observing the presence or absence of binding between each probe and a macromolecule within the biological sample.

21. The method of claim 20, wherein the probe is an oligonucleotide or an oligonucleotide derivative.

22. The method of claim 21, wherein the probe is an oligonucleotide capable of priming polynucleotide synthesis in a polymerase chain reaction.

23. The method of claim 22, wherein the probe is an antibody or an antibody derivative.

24. A method of treating an individual shown to have an increased risk for developing AMD for age-related macular degeneration, comprising administering to the individual a pharmaceutically effective amount of a composition, wherein said individual is characterized in that a biological sample obtained from said individual exhibits the allelic variant of claim 1, and wherein said composition comprises an agent to cancel the association between said allelic variant and development of AMD.

25. The method of claim 24, wherein the agent is a protective variant of the recombinant CFH gene.

26. The method of claim 25, wherein the agent is an RNA complementary to at least a portion of the nucleotide sequence of the allelic variant.

27. The method of claim 26, wherein the RNA is antisense RNA.

28. The method of claim 26, wherein the RNA is a ribozyme.

29. The method of claim 26, wherein the RNA is a short interfering RNA (siRNA).

30. A kit for use in determining an individual's susceptibility to age-related macular degeneration (AMD), said kit comprising:
(a) a set of instructions that set forth a protocol for testing a biological sample obtained from the individual for the presence or absence of an allelic variant of the Complement Factor H(CFH) gene, wherein the allelic variant is the allelic variant of claim 1; and
(b) one or more probes, wherein each of said one or more probes is characterized by its ability to bind with specificity to said allelic variant.

31. A kit for use in determining an individual's susceptibility to age-related macular degeneration (AMD), said kit comprising:
(a) a set of instructions that set forth a protocol for testing a biological sample obtained from the individual for the presence or absence of a polymorphic haplotype, wherein the haplotype is the haplotype of claim 11; and
(b) one or more probes, wherein each of said one or more probes is characterized by its ability to bind with specificity to at least one of said polymorphic sites.


SEQUENCE LISTING (308 sequences)

SEQ ID NO: 1 - DNA, 201 bp, Homo sapiens
aaatacagaaattctgacaggttcctggtctgaccaaacatatccagaaggcacccaggctatctataaatgccgccctggatatagatctcttggaaatataataatggtatgcaggaagggagaatgggttgctcttaatccattaaggaaatgtcagagtaagtacttaatacatttgtgaaatttatgaaaactagg

SEQ ID NO: 2 - DNA, 201 bp, Homo sapiens
cctagttttcataaatttcacaaatgtattaagtacttactctgacatttccttaatggattaagagcaacccattctcccttcctgcataccattattatatttccaagagatctatatccagggcggcatttatagatagcctgggtgccttctggatatgtttggtcagaccaggaacctgtcagaatttctgtattt

SEQ ID NO: 3 - DNA, 602 bp, Homo sapiens
gctgggcaggctggtctcgaactcctggcctcgtgatccacccacctcagcctcccaaagtgctgggattacaggcgtgagccaccgtgcccagccaatacatcatcattttcaaaaaggggtggtcatcctccaaaattaaaaaagcaagcatatagtttaagttcaattatgaaataatggctttgctatgtttaattttccttacattcaatctgtcttcttatataatatcaaatatacttgttcccccactcctacataaaatatattccttgctattacatactaattcataactttttttttttcgttttagaaaggccctgtggacatcctggagatactccttttggtacttttacccttacaggaggaaatgtgtttgaatatggtgtaaaagctgtgtatacatgtaatgaggggtatgtagtccatacgaaaagaggtttataattaagatagtaaataggaactctactactttatatatttttaaggttattatatttttctatgagcatttaaaaaagtaatacacaagtacctgaaagtttaactatgatggaaataattaaatctggataccatattatctcctt

SEQ ID NO: 4 - DNA, 602 bp, Homo sapiens
aaggagataatatggtatccagatttaattatttccatcatagttaaactttcaggtacttgtgtattacttttttaaatgctcatagaaaaatataataaccttaaaaatatataaagtagtagagttcctatttactatcttaattataaacctcttttcgtatggactacatacccctcattacatgtatacacagcttttacaccatattcaaacacatttcctcctgtaagggtaaaagtaccaaaaggagtatctccaggatgtccacagggcctttctaaaacgaaaaaaaaaaagttatgaattagtatgtaatagcaaggaatatattttatgtaggagtgggggaacaagtatatttgatattatataagaagacagattgaatgtaaggaaaattaaacatagcaaagccattatttcataattgaacttaaactatatgcttgcttttttaattttggaggatgaccacccctttttgaaaatgatgatgtattggctgggcacggtggctcacgcctgtaatcccagcactttgggaggctgaggtgggtggatcacgaggccaggagttcgagaccagcctgcccagc

SEQ ID NO: 5 - DNA, 201 bp, Homo sapiens
attattttaaacacttaggtcgaagaggatggaaaattaacgatgatttctctattgaagttaatgtcataaagtttgcttttacatcttttaggaacacagtgttatattattctagggcataaatgaaaatgtatttaattatctcaagctttatatttcttaattatttaaaaatactagtttgttactacaaaatac

SEQ ID NO: 6 - DNA, 201 bp, Homo sapiens
gtattttgtagtaacaaactagtatttttaaataattaagaaatataaagcttgagataattaaatacattttcatttatgccctagaataatataacactgtgttcctaaaagatgtaaaagcaaactttatgacattaacttcaatagagaaatcatcgttaattttccatcctcttcgacctaagtgtttaaaataat

SEQ ID NO: 7 - DNA, 201 bp, Homo sapiens
atggtgactactcacctttaaggattaaacacagaactggagatgaaatcacgtaccagtgtagaaatggtttttatcctgcaacccggggaaatacagcaaaatgcacaagtactggctggatacctgctccgagatgtacctgtaagttccattcatatcttgacccatttcttaattctgaaatttcttttaaacaca

SEQ ID NO: 8 - DNA, 201 bp, Homo sapiens
tgtgtttaaaagaaatttcagaattaagaaatgggtcaagatatgaatggaacttacaggtacatctcggagcaggtatccagccagtacttgtgcattttgctgtatttccccgggttgcaggataaaaaccatttctacactggtacgtgatttcatctccagttctgtgtttaatccttaaaggtgagtagtcaccat

SEQ ID NO: 9 - DNA, 201 bp, Homo sapiens
aatacatgagctaagcggtaaaattggcatatttctcaaccgaaaagcttatttaattaaccagggatagggtatgtgtatttaagggaaatatattaaataggtctgtgcatttttctttctggagagccttcatacaaatttacgcatcatgtgatccacaagacataatttcatcttcattaacaaagaccttcttgt

SEQ ID NO: 10 - DNA, 201 bp, Homo sapiens
acaagaaggtctttgttaatgaagatgaaattatgtcttgtggatcacatgatgcgtaaatttgtatgaaggctctccagaaagaaaaatgcacagacctatttaatatatttcccttaaatacacataccctatccctggttaattaaataagcttttcggttgagaaatatgccaattttaccgcttagctcatgtatt

SEQ ID NO: 11 - DNA, 201 bp, Homo sapiens
tgagttctatcatttgttttgacctagaaaccctaatggaatgtgtaattatactaagaagagaatataattcagtgataaaaatttatctctaatatgagtgtttattacagtaaaatttctttatactttttttaaaatttttattgcaagtgaaaccttgtgattatccagacattaaacatggaggtctatatcatg

SEQ ID NO: 12 - DNA, 201 bp, Homo sapiens
catgatatagacctccatgtttaatgtctggataatcacaaggtttcacttgcaataaaaattttaaaaaaagtataaagaaattttactgtaataaacactcatattagagataaatttttatcactgaattatattctcttcttagtataattacacattccattagggtttctaggtcaaaacaaatgatagaactca

SEQ ID NO: 13 - DNA, 201 bp, Homo sapiens
gagcaaatttatgtttctcatttactttatttatttatcattgttatggtccttaggaaaatgttattttccttatttggaaaatggatataatcaaaatcatggaagaaagtttgtacagggtaaatctatagacgttgcctgccatcctggctacgctcttccaaaagcgcagaccacagttacatgtatggagaatgg

SEQ ID NO: 14 - DNA, 201 bp, Homo sapiens
ccattctccatacatgtaactgtggtctgcgcttttggaagagcgtagccaggatggcaggcaacgtctatagatttaccctgtacaaactttcttccataattttgattatatccattttccaaataaggaaaataacattttcctaaggaccataacaatgataaataaataaagtaaatgagaaacataaatttgctc

SEQ ID NO: 15 - DNA, 201 bp, Homo sapiens
catctatggtagaaccacctgggcccccaaaggccaataaaaacggacatcccattgcaggtcccaaccagtgtgcttactgcaacaggggggacactgagtggaaaatttctcatgccttacaaagcctgatgttaaacagtcagccttctgcccaccaaatgcctgggatagctggggagcttgagagagatactaaag

SEQ ID NO: 16 - DNA, 201 bp, Homo sapiens
ctttagtatctctctcaagctccccagctatcccaggcatttggtgggcagaaggctgactgtttaacatcaggctttgtaaggcatgagaaattttccactcagtgtcccccctgttgcagtaagcacactggttgggacctgcaatgggatgtccgtttttattggcctttgggggcccaggtggttctaccatagatg

SEQ ID NO: 17 - DNA, 201 bp, Homo sapiens
tattctcttcccttttagaaacatgttccaaatcaagtatagatattgagaatgggtttatttctgaatctcagtatacatatgccttaaaagaaaaagcaaaatatcaatgcaaactaggatatgtaacagcagatggtgaaacatcaggatcaattacatgtgggaaagatggatggtcagctcaacccacgtgcatta

SEQ ID NO: 18 - DNA, 201 bp, Homo sapiens
taatgcacgtgggttgagctgaccatccatctttcccacatgtaattgatcctgatgtttcaccatctgctgttacatatcctagtttgcattgatattttgctttttcttttaaggcatatgtatactgagattcagaaataaacccattctcaatatctatacttgatttggaacatgtttctaaaagggaagagaata

SEQ ID NO: 19 - DNA, 201 bp, Homo sapiens
tgttgtgtagaaagatatactttcacttttgacaactattttacgacaacaaattctcaccagtcatagattatttttgtacggtacctatttattagtagatctaatcaataaagctttttcttcttagaatgggaaatactcagattgtttattagatgacattagaaatgacattctaaattttttatgcactagaat

SEQ ID NO: 20 - DNA, 201 bp, Homo sapiens
attctagtgcataaaaaatttagaatgtcatttctaatgtcatctaataaacaatctgagtatttcccattctaagaagaaaaagctttattgattagatctactaataaataggtaccgtacaaaaataatctatgactggtgagaatttgttgtcgtaaaatagttgtcaaaagtgaaagtatatctttctacacaaca

SEQ ID NO: 21 - DNA, 201 bp, Homo sapiens
acagttattgatctttctatttattctattttaatcatataaattatttttcatcaaaaattctaattttaatatttttattttttattttttattataacattaattatatttttaatattttttagtggcacaaatacaattatgcccacctccacctcagattcccaattctcacaatatgacaaccacactgaatta

SEQ ID NO: 22 - DNA, 201 bp, Homo sapiens
taattcagtgtggttgtcatattgtgagaattgggaatctgaggtggaggtgggcataattgtatttgtgccactaaaaaatattaaaaatataattaatgttataataaaaaataaaaaataaaaatattaaaattagaatttttgatgaaaaataatttatatgattaaaatagaataaatagaaagatcaataactgt

SEQ ID NO: 23 - DNA, 201 bp, Homo sapiens
tttttaatagatttagaagaatttaatgtaattaagacaaaatggctaatatattttctcaagttataagaaaaatgttgtacagtattcattgattctacatatcgctattttagaatccattacatgtattgtatgtaacctatttttaaagatttgcggaacaaatacatatttttcctatttcagaaacagattgtc

SEQ ID NO: 24 - DNA, 201 bp, Homo sapiens
gacaatctgtttctgaaataggaaaaatatgtatttgttccgcaaatctttaaaaataggttacatacaatacatgtaatggattctaaaatagcgatatgtagaatcaatgaatactgtacaacatttttcttataacttgagaaaatatattagccattttgtcttaattacattaaattcttctaaatctattaaaaa

SEQ ID NO: 25 - DNA, 201 bp, Homo sapiens
ggcctcaagtgatccacctgccttggcctccctaagtgctgggatttcaggcatgagccatggcaactggcctgctctgttctaaatgcagatctaaaccccctgcaggtaacctggatgaggacatcattgcagaagagaacatcgtttcccgaagtgagttcccagagagctggctgtggaacgttgaggacttgaaag

SEQ ID NO: 26 - DNA, 201 bp, Homo sapiens
ctttcaagtcctcaacgttccacagccagctctctgggaactcacttcgggaaacgatgttctcttctgcaatgatgtcctcatccaggttacctgcagggggtttagatctgcatttagaacagagcaggccagttgccatggctcatgcctgaaatcccagcacttagggaggccaaggcaggtggatcacttgaggcc

SEQ ID NO: 27 - DNA, 201 bp, Homo sapiens
atcgtttcccgaagtgagttcccagagagctggctgtggaacgttgaggacttgaaagagccaccgaaaaatgggtaaggccggggtacccccggtacaagccaccccagagtcagaccgtttaatttgcatgcacctgctatctctggtcttctctggaatcacagtgcaaccccacagcccaacctagaaaaacctgct

SEQ ID NO: 28 - DNA, 201 bp, Homo sapiens
agcaggtttttctaggttgggctgtggggttgcactgtgattccagagaagaccagagatagcaggtgcatgcaaattaaacggtctgactctggggtggcttgtaccgggggtaccccggccttacccatttttcggtggctctttcaagtcctcaacgttccacagccagctctctgggaactcacttcgggaaacgat

SEQ ID NO: 29 - DNA, 201 bp, Homo sapiens
acctacatggaggcacccccagacccttccagcctgtcccttggggtccctctgcaccagttcttcccctctaccaccctgctagatgacatctcctaataccccaacctcttctccatccagaatctctacgaagctcatgaatatatttttgaaagactccatcaccacgtgggagattctggctgtgagcatgtcgga

SEQ ID NO: 30 - DNA, 201 bp, Homo sapiens
tccgacatgctcacagccagaatctcccacgtggtgatggagtctttcaaaaatatattcatgagcttcgtagagattctggatggagaagaggttggggtattaggagatgtcatctagcagggtggtagaggggaagaactggtgcagagggaccccaagggacaggctggaagggtctgggggtgcctccatgtaggt

SEQ ID NO: 31 - DNA, 201 bp, Homo sapiens
cttcagcaaagacaggctgagttagggagagctcctgcggagtggactaagagctgagacccaggagcctggccttgtccactccccgaccttgacactccgtgttctgtctctgcccgagcagggatctgtgtggcagaccccttcgaggtcacagtaatgcaggacttcttcatcgacctgcggctaccctactctgtt

SEQ ID NO: 32 - DNA, 201 bp, Homo sapiens
aacagagtagggtagccgcaggtcgatgaagaagtcctgcattactgtgacctcgaaggggtctgccacacagatccctgctcgggcagagacagaacacggagtgtcaaggtcggggagtggacaaggccaggctcctgggtctcagctcttagtccactccgcaggagctctccctaactcagcctgtctttgctgaag

SEQ ID NO: 33 - DNA, 201 bp, Homo sapiens
accagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtggtgagtgcttggggcacccacaaacccttgtccttcagagagggctcct

SEQ ID NO: 34 - DNA, 201 bp, Homo sapiens
aggagccctctctgaaggacaagggtttgtgggtgccccaagcactcaccacgaccttcagggacttcctgacaccgtcactgatgaaatgatggtagacagcagccttgacttccacttcctgcaggccggtctttagcggcacgatgacatatggaacggacaacgaggacttgggggggatggttacggtctgctggt

SEQ ID NO: 35 - DNA, 201 bp, Homo sapiens
gggagagaggggtccaggagggatttttcacaggctccacctttccccagctggccaatggtgttgacagatacatctccaagtatgagctggacaaagctttctccgataggaacaccctcatcatctacctggacaaggtaaggctgcatcatcctcccctgggaggcttccaggggcaccctgacctctatctggctg

SEQ ID NO: 36 - DNA, 201 bp, Homo sapiens
cagccagatagaggtcagggtgcccctggaagcctcccaggggaggatgatgcagccttaccttgtccaggtagatgatgagggtgttcctatcggagaaagctttgtccagctcatacttggagatgtatctgtcaacaccattggccagctggggaaaggtggagcctgtgaaaaatccctcctggacccctctctccc

SEQ ID NO: 37 - DNA, 201 bp, Homo sapiens
catctccaggtctgaagactgagaactggggatttgggcctcccaggcgtggtctttggagggaggcccttatcctctcatcttcacatcacatctgcccgcagaggaaagctgtacccggttctaccatccggaaaaggaggatggaaagctgaacaagctctgccgtgatgaactgtgccgctgtgctgagggtgagtt

SEQ ID NO: 38 - DNA, 201 bp, Homo sapiens
aactcaccctcagcacagcggcacagttcatcacggcagagcttgttcagctttccatcctccttttccggatggtagaaccgggtacagctttcctctgcgggcagatgtgatgtgaagatgagaggataagggcctccctccaaagaccacgcctgggaggcccaaatccccagttctcagtcttcagacctggagatg

SEQ ID NO: 39 - DNA, 201 bp, Homo sapiens
ctgaacaagctctgccgtgatgaactgtgccgctgtgctgagggtgagttccctggagccgggaacaggtgggtctgagcaagccacacttacccaggtcatctatcccatggtcagggacccccagacccatacccaggggataccaaggggggtaggctcccagggctggccacacccatgggcagtaggccccagata

SEQ ID NO: 40 - DNA, 201 bp, Homo sapiens
tatctggggcctactgcccatgggtgtggccagccctgggagcctaccccccttggtatcccctgggtatgggtctgggggtccctgaccatgggatagatgacctgggtaagtgtggcttgctcagacccacctgttcccggctccagggaactcaccctcagcacagcggcacagttcatcacggcagagcttgttcag

SEQ ID NO: 41 - DNA, 201 bp, Homo sapiens
actttctgcttgggagagagagccacatgcccgggtgcacttgcaaaccagggtgcccctcatggtcaacctagcccaccacccaaactgtctgcctctctcccacagtgtacaagacccgactggtcaaggttcagctgtccaatgactttgacgagtacatcatggccattgagcagaccatcaagtcaggtcaggctc

SEQ ID NO: 42 - DNA, 201 bp, Homo sapiens
gagcctgacctgacttgatggtctgctcaatggccatgatgtactcgtcaaagtcattggacagctgaaccttgaccagtcgggtcttgtacactgtgggagagaggcagacagtttgggtggtgggctaggttgaccatgaggggcaccctggtttgcaagtgcacccgggcatgtggctctctctcccaagcagaaagt

SEQ ID NO: 43 - DNA, 200 bp, Homo sapiens
agtttgtctgggttttcctgtcccttgtagccagtcttcccagctgcctctgccttggcctttccaactttctattcgtcagggtgtcaagccccagaggacacagggacagagacaaaggtgtcaagtgaccttcctacccccaacagaccttctctctaatatagaggaggaaaagaagggagggtagcaggacaaga

SEQ ID NO: 44 - DNA, 201 bp, Homo sapiens
ttcttgtcctgctaccctcccttcttttcctcctctatattagagagaaggtctgttgggggtaggaaggtcacttgacacctttgtctctgtccctgtgtcctctggggcttgacaccctgacgaatagaaagttggaaaggccaaggcagaggcagctgggaagactggctacaagggacaggaaaacccagacaaact

SEQ ID NO: 45 - DNA, 20 bp, Homo sapiens
aagattccttccgctctggt

SEQ ID NO: 46 - DNA, 20 bp, Homo sapiens
ccccatccctgtcctagaat

SEQ ID NO: 47 - DNA, 20 bp, Homo sapiens
gcatgtgtggatgtgtgtga

SEQ ID NO: 48 - DNA, 20 bp, Homo sapiens
gcagacggactcctgattct

SEQ ID NO: 49 - DNA, 20 bp, Homo sapiens
tgcttcattgccctttatcc

SEQ ID NO: 50 - DNA, 20 bp, Homo sapiens
cctgacaccacctggaagag

SEQ ID NO: 51 - DNA, 20 bp, Homo sapiens
ctgtccatcgtctcctcctc

SEQ ID NO: 52 - DNA, 21 bp, Homo sapiens
gcctgactcttaagcactgga

SEQ ID NO: 53 - DNA, 20 bp, Homo sapiens
aaattctgggaaaggggatg

SEQ ID NO: 54 - DNA, 20 bp, Homo sapiens
aaggaactgggagggagaga

SEQ ID NO: 55 - DNA, 20 bp, Homo sapiens
gctcagcacccaaaagaaag

SEQ ID NO: 56 - DNA, 18 bp, Homo sapiens
ccccaaccctctcattcc

SEQ ID NO: 57 - DNA, 20 bp, Homo sapiens
gcaacggtgtacatcctcct

SEQ ID NO: 58 - DNA, 20 bp, Homo sapiens
acacggtgtctgagtggtca

SEQ ID NO: 59 - DNA, 19 bp, Homo sapiens
atccatggtgaggctcctg

SEQ ID NO: 60 - DNA, 20 bp, Homo sapiens
gtcagccccagacagacact

SEQ ID NO: 61 - DNA, 18 bp, Homo sapiens
aatgccagcgctgtgttc

SEQ ID NO: 62 - DNA, 20 bp, Homo sapiens
acaggttggctgtatgcgac

SEQ ID NO: 63 - DNA, 22 bp, Homo sapiens
caacagcctgatcagattcaac

SEQ ID NO: 64 - DNA, 20 bp, Homo sapiens
ggtggggagaatggtctagg

SEQ ID NO: 65 - DNA, 19 bp, Homo sapiens
ctgctcttccccgactagg

SEQ ID NO: 66 - DNA, 20 bp, Homo sapiens
tggctgatgcaaagttgaag

SEQ ID NO: 67 - DNA, 20 bp, Homo sapiens
agtaacctcagcccctctcc

SEQ ID NO: 68 - DNA, 20 bp, Homo sapiens
tgccagttgatgtgttgtca

SEQ ID NO: 69 - DNA, 18 bp, Homo sapiens
cgccacatgatgctcact

SEQ ID NO: 70 - DNA, 20 bp, Homo sapiens
acgcaaagctcacacttacg

SEQ ID NO: 71 - DNA, 20 bp, Homo sapiens
atggatgcagatggaggaat

SEQ ID NO: 72 - DNA, 20 bp, Homo sapiens
ccagtgcttttgcagtctca

SEQ ID NO: 73 - DNA, 22 bp, Homo sapiens
ccttcgttttacagatgggaga

SEQ ID NO: 74 - DNA, 21 bp, Homo sapiens
aggcatagtcacagaaggtga

SEQ ID NO: 75 - DNA, 21 bp, Homo sapiens
tgcccggtctctactacttca

SEQ ID NO: 76 - DNA, 23 bp, Homo sapiens
catgtaaccaacatttctgagca

SEQ ID NO: 77 - DNA, 20 bp, Homo sapiens
gagcaacttggacactgctg

SEQ ID NO: 78 - DNA, 20 bp, Homo sapiens
caatacgcctctcaaccctc

SEQ ID NO: 79 - DNA, 21 bp, Homo sapiens
aacaatgtgtttgaatggtcc

SEQ ID NO: 80 - DNA, 20 bp, Homo sapiens
agtgcttgatgaatttgggg

SEQ ID NO: 81 - DNA, 23 bp, Homo sapiens
aaaatcagcatttcaatttgttg

SEQ ID NO: 82 - DNA, 20 bp, Homo sapiens
cccaattctcactgcactcc

SEQ ID NO: 83 - DNA, 21 bp, Homo sapiens
tgctgatttgcacataccaag

SEQ ID NO: 84 - DNA, 22 bp, Homo sapiens
tcctgtgaaaagcatcattagc

SEQ ID NO: 85 - DNA, 19 bp, Homo sapiens
ttagatagacctgtgactg

SEQ ID NO: 86 - DNA, 17 bp, Homo sapiens
tcaggcataattgctac

SEQ ID NO: 87 - DNA, 24 bp, Homo sapiens
tcaattatgaaataatggctttgc

SEQ ID NO: 88 - DNA, 25 bp, Homo sapiens
tggtatccagatttaattatttcca

SEQ ID NO: 89 - DNA, 21 bp, Homo sapiens
tgcatatgctgttcattttcc

SEQ ID NO: 90 - DNA, 27 bp, Homo sapiens
aaaaagactagattcccactctacatt

SEQ ID NO: 91 - DNA, 18 bp, Homo sapiens
tccaatcttatcctgagg

SEQ ID NO: 92 - DNA, 20 bp, Homo sapiens
aagttaaaatgaaatgatgc

SEQ ID NO: 93 - DNA, 23 bp, Homo sapiens
ggttgactgatttacctgatgga

SEQ ID NO: 94 - DNA, 23 bp, Homo sapiens
gaaccttgaacacagaaaatgct

SEQ ID NO: 95 - DNA, 25 bp, Homo sapiens
ggacaaataaataacacccactttt

SEQ ID NO: 96 - DNA, 21 bp, Homo sapiens
tgcttccaacagccttacttt

SEQ ID NO: 97 - DNA, 26 bp, Homo sapiens
tctgagttctatcatttgttttgacc

SEQ ID NO: 98 - DNA, 22 bp, Homo sapiens
tcttttcttggccctatttctg

SEQ ID NO: 99 - DNA, 23 bp, Homo sapiens
ggtttcttcttgaaaatcacagg

SEQ ID NO: 100 - DNA, 22 bp, Homo sapiens
ccattggtaaaacaaggtgaca

SEQ ID NO: 101 - DNA, 20 bp, Homo sapiens
ctttttcttattctcttccc

SEQ ID NO: 102 - DNA, 18 bp, Homo sapiens
cccacaaaaagactaaag

SEQ ID NO: 103 - DNA, 17 bp, Homo sapiens
gggaaatactcagattg

SEQ ID NO: 104 - DNA, 19 bp, Homo sapiens
gtttatgtcaaatcaggag

SEQ ID NO: 105 - DNA, 20 bp, Homo sapiens
ttacagggaaaaggatttat

SEQ ID NO: 106 - DNA, 17 bp, Homo sapiens
agattacaggcaatggg

SEQ ID NO: 107 - DNA, 17 bp, Homo sapiens
ttgattgtttaggatgc

SEQ ID NO: 108 - DNA, 20 bp, Homo sapiens
aaagtttattgtgaaaacat

SEQ ID NO: 109 - DNA, 20 bp, Homo sapiens
taccagtgtgtattggtaat

SEQ ID NO: 110 - DNA, 15 bp, Homo sapiens
tggaaatgttgaggc

SEQ ID NO: 111 - DNA, 19 bp, Homo sapiens
agttggtttgattcctatc

SEQ ID NO: 112 - DNA, 20 bp, Homo sapiens
ggaacagtgttttcattagt

SEQ ID NO: 113 - DNA, 26 bp, Homo sapiens
caagccaaaagttctattgtttaatg

SEQ ID NO: 114 - DNA, 23 bp, Homo sapiens
ttgtttacacgaagcacaagaga

SEQ ID NO: 115 - DNA, 23 bp, Homo sapiens
tggaggaatatatctttgcgagt

SEQ ID NO: 116 - DNA, 22 bp, Homo sapiens
tcaattattcccctcactttga

SEQ ID NO: 117 - DNA, 22 bp, Homo sapiens
gttggtgacagtccgatagaca

SEQ ID NO: 118 - DNA, 20 bp, Homo sapiens
aatttcccacagcagtccag

SEQ ID NO: 119 - DNA, 24 bp, Homo sapiens
aatgtaattaagacaaaatggcta

SEQ ID NO: 120 - DNA, 19 bp, Homo sapiens
cgaactcctgacctcaagt

SEQ ID NO: 121 - DNA, 21 bp, Homo sapiens
ctactcaaaatgaacactagg

SEQ ID NO: 122 - DNA, 20 bp, Homo sapiens
accctattacttgtgttctg

SEQ ID NO: 123 - DNA, 15 bp, Homo sapiens
gtgtttgcgtttgcc

SEQ ID NO: 124 - DNA, 17 bp, Homo sapiens
gagatttttccagccac

SEQ ID NO: 125 - DNA, 20 bp, Homo sapiens
ggtttggatagtgttttgag

SEQ ID NO: 126 - DNA, 17 bp, Homo sapiens
accgttagttttccagg

SEQ ID NO: 127 - DNA, 21 bp, Homo sapiens
tgatgaacccaactgtgtcag

SEQ ID NO: 128 - DNA, 21 bp, Homo sapiens
tttggctgaaatccagagaga

SEQ ID NO: 129 - DNA, 29 bp, Homo sapiens
aaattctttcagagttcaaaagtacaaag

SEQ ID NO: 130 - DNA, 30 bp, Homo sapiens
atcaagatattaacataaaacatttgttgc

SEQ ID NO: 131 - DNA, 20 bp, Homo sapiens
cttgaagccaccagacaaca

SEQ ID NO: 132 - DNA, 27 bp, Homo sapiens
tggcatacaaatacccttttatatcat

SEQ ID NO: 133 - DNA, 21 bp, Homo sapiens
tcgtcatgatgttcaaagctc

SEQ ID NO: 134 - DNA, 20 bp, Homo sapiens
gggttccaagtgttgggtaa

SEQ ID NO: 135 - DNA, 20 bp, Homo sapiens
cttgcccaagctgtaactcc

SEQ ID NO: 136 - DNA, 19 bp, Homo sapiens
gcaacgaggcatcaatcat

SEQ ID NO: 137 - DNA, 24 bp, Homo sapiens
tcttaatgactccattatcccaaa

SEQ ID NO: 138 - DNA, 20 bp, Homo sapiens
ggcatgctgtgcaaacataa

SEQ ID NO: 139 - DNA, 21 bp, Homo sapiens
tgtggagaccaaagtgatgaa

SEQ ID NO: 140 - DNA, 21 bp, Homo sapiens
cacccatctatgttcccctta

SEQ ID NO: 141 - DNA, 23 bp, Homo sapiens
gaaataaggtgcaatggttttca

SEQ ID NO: 142 - DNA, 21 bp, Homo sapiens
agaaaaggcctctacgcctaa

SEQ ID NO: 143 - DNA, 23 bp, Homo sapiens
gggaggataagttttaaggcaga

SEQ ID NO: 144 - DNA, 24 bp, Homo sapiens
ccaaaactacttgttgcttgaatc

SEQ ID NO: 145 - DNA, 21 bp, Homo sapiens
tggctgtgactctcctgtttt

SEQ ID NO: 146 - DNA, 22 bp, Homo sapiens
tgctttatcatctgccacaatc

SEQ ID NO: 147 - DNA, 20 bp, Homo sapiens
ttctggggaaatgaaaaagg

SEQ ID NO: 148 - DNA, 23 bp, Homo sapiens
gcttctctctgagtgctaggaaa

SEQ ID NO: 149 - DNA, 21 bp, Homo sapiens
ccctttcataatcccaatggt

SEQ ID NO: 150 - DNA, 20 bp, Homo sapiens
ctacagccagaaggccaaat

SEQ ID NO: 151 - DNA, 20 bp, Homo sapiens
agagcccatgctattgctgt

SEQ ID NO: 152 - DNA, 21 bp, Homo sapiens
ggcataaactctgtggagacc

SEQ ID NO: 153 - DNA, 21 bp, Homo sapiens
tctgtgggtcagggagactaa

SEQ ID NO: 154 - DNA, 20 bp, Homo sapiens
gcaccttgctcttgtgtgaa

SEQ ID NO: 155 - DNA, 20 bp, Homo sapiens
ccacttggtacaacccaaaa

SEQ ID NO: 156 - DNA, 20 bp, Homo sapiens
acacacaggaccctgacgtt

SEQ ID NO: 157 - DNA, 20 bp, Homo sapiens
gctgctgacccagctctagt

SEQ ID NO: 158 - DNA, 20 bp, Homo sapiens
caggttaagtcccaccctga

SEQ ID NO: 159 - DNA, 20 bp, Homo sapiens
tgccaaaaacagcaatttca

SEQ ID NO: 160 - DNA, 20 bp, Homo sapiens
ctatccccacagaggcttcc

SEQ ID NO: 161 - DNA, 20 bp, Homo sapiens
ggaactgggaagcttctgct

SEQ ID NO: 162 - DNA, 19 bp, Homo sapiens
aaggttgcatcctcccttg

SEQ ID NO: 163 - DNA, 20 bp, Homo sapiens
tgtttgaggtggggtttctg

SEQ ID NO: 164 - DNA, 20 bp, Homo sapiens
gttctgaggaggggaaggag

SEQ ID NO: 165 - DNA, 20 bp, Homo sapiens
tccagtttctctgcctcctc

SEQ ID NO: 166 - DNA, 20 bp, Homo sapiens
accataaccctggacgttga

SEQ ID NO: 167 - DNA, 20 bp, Homo sapiens
cctaggtggtaggtgggaag

SEQ ID NO: 168 - DNA, 20 bp, Homo sapiens
ggagaatcagaattcagggc

SEQ ID NO: 169 - DNA, 20 bp, Homo sapiens
tgaggttcccaggctaaatg

SEQ ID NO: 170 - DNA, 20 bp, Homo sapiens
ccatgggcagtagtttgacc

SEQ ID NO: 171 - DNA, 20 bp, Homo sapiens
gacagcctcctgtctcatgg

SEQ ID NO: 172 - DNA, 20 bp, Homo sapiens
gccgcttttacctctggaat

SEQ ID NO: 173 - DNA, 20 bp, Homo sapiens
attccagaggtaaaagcggc

SEQ ID NO: 174 - DNA, 21 bp, Homo sapiens
ggatctccagagcactcttcc

SEQ ID NO: 175 - DNA, 19 bp, Homo sapiens
cagggaagagtgctctgga

SEQ ID NO: 176 - DNA, 19 bp, Homo sapiens
agccagacctggagaagga

SEQ ID NO: 177 - DNA, 20 bp, Homo sapiens
aacctcccaaagtgctgaga

SEQ ID NO: 178 - DNA, 20 bp, Homo sapiens
caacctccctagatccctca

SEQ ID NO: 179 - DNA, 20 bp, Homo sapiens
aggttggggcttacagttgg

SEQ ID NO: 180 - DNA, 18 bp, Homo sapiens
caaggccagccctacctg

SEQ ID NO: 181 - DNA, 20 bp, Homo sapiens
agtgatctgcctgcctcaac

SEQ ID NO: 182 - DNA, 20 bp, Homo sapiens
tctccgctcaaggaaaactg

SEQ ID NO: 183 - DNA, 20 bp, Homo sapiens
gaaggccacctgtgtctctg

SEQ ID NO: 184 - DNA, 19 bp, Homo sapiens
atgtgaaagtctcgtggcg

SEQ ID NO: 185 - DNA, 20 bp, Homo sapiens
cccagccagttctctccttt

SEQ ID NO: 186 - DNA, 21 bp, Homo sapiens
gggaaattccatgtgaaagtg

SEQ ID NO: 187 - DNA, 20 bp, Homo sapiens
cacacaagctgggaaatcct

SEQ ID NO: 188 - DNA, 20 bp, Homo sapiens
cagcccccagccttttatac

SEQ ID NO: 189 - DNA, 20 bp, Homo sapiens
tgggacttctgcagtttctg

SEQ ID NO: 190 - DNA, 18 bp, Homo sapiens
aaaggccaaggagggatg

SEQ ID NO: 191 - DNA, 20 bp, Homo sapiens
agcagacaagcaaagcaagc

SEQ ID NO: 192 - DNA, 20 bp, Homo sapiens
gtttctgccttaggccactg

SEQ ID NO: 193 - DNA, 20 bp, Homo sapiens
gaatgcgctgtttctcagtg

SEQ ID NO: 194 - DNA, 20 bp, Homo sapiens
cccgtgagcaaggtagagag

SEQ ID NO: 195 - DNA, 20 bp, Homo sapiens
ggctccggacactgtaactc

SEQ ID NO: 196 - DNA, 20 bp, Homo sapiens
gtgaggaaagagcagggttg

SEQ ID NO: 197 - DNA, 20 bp, Homo sapiens
cccgtctctttggtcactgt

SEQ ID NO: 198 - DNA, 19 bp, Homo sapiens
tcagaggggctggaatgat

SEQ ID NO: 199 - DNA, 20 bp, Homo sapiens
ctcagcccttgagcctctta

SEQ ID NO: 200 - DNA, 20 bp, Homo sapiens
ctgtcgccctcaaggtagtc

SEQ ID NO: 201 - DNA, 20 bp, Homo sapiens
tgtgggatttcagttgcaga

SEQ ID NO: 202 - DNA, 20 bp, Homo sapiens
ccccaaccatgggtatagtg

SEQ ID NO: 203 - DNA, 20 bp, Homo sapiens
ccttcctcaacttgctcacc

SEQ ID NO: 204 - DNA, 20 bp, Homo sapiens
caggggaacctgaggagagt

SEQ ID NO: 205 - DNA, 20 bp, Homo sapiens
gactcctacccaaaaggctg

SEQ ID NO: 206 - DNA, 20 bp, Homo sapiens
agaaagtgggaggtgttgcc

SEQ ID NO: 207 - DNA, 20 bp, Homo sapiens
tgggggaagacgtgaagtta

SEQ ID NO: 208 - DNA, 20 bp, Homo sapiens
cctattgcacctccctctca

SEQ ID NO: 209 - DNA, 19 bp, Homo sapiens
aaggcaatggggagatgac

SEQ ID NO: 210 - DNA, 20 bp, Homo sapiens
acatgcattgagctttcctg

SEQ ID NO: 211 - DNA, 20 bp, Homo sapiens
tgagctgggtccctagtctg

SEQ ID NO: 212 - DNA, 18 bp, Homo sapiens
cagtgcctgttccccact

SEQ ID NO: 213 - DNA, 19 bp, Homo sapiens
aggctccacccagtttgtc

SEQ ID NO: 214 - DNA, 18 bp, Homo sapiens
agaccctcgggcctctgt

SEQ ID NO: 215 - DNA, 20 bp, Homo sapiens
ggactctgggtgtggtgaag

SEQ ID NO: 216 - DNA, 19 bp, Homo sapiens
gagagacacccccttcctg

SEQ ID NO: 217 - DNA, 20 bp, Homo sapiens
cccagagatgggattcttgc

SEQ ID NO: 218 - DNA, 18 bp, Homo sapiens
gcccaggagaacctgcac

SEQ ID NO: 219 - DNA, 18 bp, Homo sapiens
ctggaggacgcgtgagtg

SEQ ID NO: 220 - DNA, 18 bp, Homo sapiens
gagggtgtgggggttgtg

SEQ ID NO: 221 - DNA, 21 bp, Homo sapiens
aaatctctcctgctgcactga

SEQ ID NO: 222 - DNA, 19 bp, Homo sapiens
ctgggccctgttcctactt

SEQ ID NO: 223 - DNA, 20 bp, Homo sapiens
aattaacacgggagggatga

SEQ ID NO: 224 - DNA, 20 bp, Homo sapiens
ccaattgcccagctaatttt

SEQ ID NO: 225 - DNA, 20 bp, Homo sapiens
ggtcacccaagcaacaaagt

SEQ ID NO: 226 - DNA, 20 bp, Homo sapiens
ggtcaggctgatcaggaact

SEQ ID NO: 227 - DNA, 21 bp, Homo sapiens
tgggaggaagaccacctttac

SEQ ID NO: 228 - DNA, 19 bp, Homo sapiens
agcccagcacagaaactcc

SEQ ID NO: 229 - DNA, 20 bp, Homo sapiens
gaagttttccctgaccctcc

SEQ ID NO: 230 - DNA, 20 bp, Homo sapiens
tccatggggtgagtaacctg

SEQ ID NO: 231 - DNA, 20 bp, Homo sapiens
tgggtccaacagagaaaggt

SEQ ID NO: 232 - DNA, 19 bp, Homo sapiens
acccccaaatgtctgcttc

SEQ ID NO: 233 - DNA, 20 bp, Homo sapiens
ggcagtgcccagagaagata

SEQ ID NO: 234 - DNA, 19 bp, Homo sapiens
gcagggcttagaaagggag

SEQ ID NO: 235 - DNA, 20 bp, Homo sapiens
aagaataatgggcaggcaag

SEQ ID NO: 236 - DNA, 20 bp, Homo sapiens
ttgcctctcctaagcctgtg

SEQ ID NO: 237 - DNA, 19 bp, Homo sapiens
cacctgggtccctgttctt

SEQ ID NO: 238 - DNA, 18 bp, Homo sapiens
ggagagatggcgttggtg

SEQ ID NO: 239 - DNA, 18 bp, Homo sapiens
cttgaggagggggctcag

SEQ ID NO: 240 - DNA, 18 bp, Homo sapiens
gtcccccacctggtcttc

SEQ ID NO: 241 - DNA, 19 bp, Homo sapiens
gtcttcagcacaggcagga

SEQ ID NO: 242 - DNA, 19 bp, Homo sapiens
gacctggtctcccctctca

SEQ ID NO: 243 - DNA, 20 bp, Homo sapiens
aagttggggtccattctgag

SEQ ID NO: 244 - DNA, 18 bp, Homo sapiens
gacaggaccccactgtgc

SEQ ID NO: 245 - DNA, 20 bp, Homo sapiens
caccaattcccaggtctcag

SEQ ID NO: 246 - DNA, 18 bp, Homo sapiens
gggtgagtggcagggaac

SEQ ID NO: 247 - DNA, 20 bp, Homo sapiens
cctcctccttgtttcttccg

SEQ ID NO: 248 - DNA, 23 bp, Homo sapiens
agacagttgagagacagagaggg

SEQ ID NO: 249 - DNA, 20 bp, Homo sapiens
cccccaacctttctgtcttt

SEQ ID NO: 250 - DNA, 20 bp, Homo sapiens
aaccatccctgtgtctggtc

SEQ ID NO: 251 - DNA, 20 bp, Homo sapiens
gagatgagagtgggacaggg

SEQ ID NO: 252 - DNA, 20 bp, Homo sapiens
tactaggtggtggtggaggc

SEQ ID NO: 253 - DNA, 20 bp, Homo sapiens
atcaaatgggagcaggaaca

SEQ ID NO: 254 - DNA, 19 bp, Homo sapiens
cttggggtacttgccgact

SEQ ID NO: 255 - DNA, 18 bp, Homo sapiens
gtgcaggagccagagagg

SEQ ID NO: 256 - DNA, 18 bp, Homo sapiens
caccagacagggcatcct

SEQ ID NO: 257 - DNA, 19 bp, Homo sapiens
tgcccagccttaatttttg

SEQ ID NO: 258 - DNA, 20 bp, Homo sapiens
ggttgcactgtgattccaga

SEQ ID NO: 259 - DNA, 19 bp, Homo sapiens
ggtccctctgcaccagttc

SEQ ID NO: 260 - DNA, 23 bp, Homo sapiens
aacatagggttgattgagtttgg

SEQ ID NO: 261 - DNA, 20 bp, Homo sapiens
cctgcggagtggactaagag

SEQ ID NO: 262 - DNA, 20 bp, Homo sapiens
acctgccagggagagaaagg

SEQ ID NO: 263 - DNA, 20 bp, Homo sapiens
atccgagccgttctctacaa

SEQ ID NO: 264 - DNA, 20 bp, Homo sapiens
cctggatctccaacctgagt

SEQ ID NO: 265 - DNA, 20 bp, Homo sapiens
cctgctgaccatctgtgtgt

SEQ ID NO: 266 - DNA, 20 bp, Homo sapiens
cgggttaaacacctccagaa

SEQ ID NO: 267 - DNA, 19 bp, Homo sapiens
tcctcgcctgtccctaacc

SEQ ID NO: 268 - DNA, 20 bp, Homo sapiens
ggatcttaggggagggatgc

SEQ ID NO: 269 - DNA, 18 bp, Homo sapiens
gtcccctcctcccagaac

SEQ ID NO: 270 - DNA, 18 bp, Homo sapiens
gctggcctaagggaccac

SEQ ID NO: 271 - DNA, 19 bp, Homo sapiens
aaggcaaaccaggcaaact

SEQ ID NO: 272 - DNA, 20 bp, Homo sapiens
gggctctctctcgtgttcat

SEQ ID NO: 273 - DNA, 20 bp, Homo sapiens
agcggggtaacacctagaag

SEQ ID NO: 274 - DNA, 20 bp, Homo sapiens
gagctgcaaattccctgaag

SEQ ID NO: 275 - DNA, 20 bp, Homo sapiens
atgctcaggaagtgctgctc

SEQ ID NO: 276 - DNA, 20 bp, Homo sapiens
ctaaaagccatgcatcccag

SEQ ID NO: 277 - DNA, 21 bp, Homo sapiens
agcatgcatctctttctgagc

SEQ ID NO: 278 - DNA, 20 bp, Homo sapiens
ctgggcctcagtgtcttctc

SEQ ID NO: 279 - DNA, 20 bp, Homo sapiens
gatgtcccagctctgatttg

SEQ ID NO: 280 - DNA, 20 bp, Homo sapiens
ctaggactgctggggacaag

SEQ ID NO: 281 - DNA, 20 bp, Homo sapiens
ggaacagaaacccacacctg

SEQ ID NO: 282 - DNA, 20 bp, Homo sapiens
tagagggatggccaagatga

SEQ ID NO: 283 - DNA, 21 bp, Homo sapiens
cctgatcagacctccttgtcc

SEQ ID NO: 284 - DNA, 21 bp, Homo sapiens
ctgacccagagacaaaagctg

SEQ ID NO: 285 - DNA, 20 bp, Homo sapiens
ttcagtttctccaccactgc

SEQ ID NO: 286 - DNA, 21 bp, Homo sapiens
gaccttcattctcagatcccc

SEQ ID NO: 287 - DNA, 20 bp, Homo sapiens
ctgaagactgagaactgggg

SEQ ID NO: 288 - DNA, 20 bp, Homo sapiens
ccatgacaaccacacctacc

SEQ ID NO: 289 - DNA, 20 bp, Homo sapiens
ttctgcttgggagagagagc

SEQ ID NO: 290 - DNA, 20 bp, Homo sapiens
gaagaatggcaggtcaggaa

SEQ ID NO: 291 - DNA, 19 bp, Homo sapiens
agtgagtgctttccctgcg

SEQ ID NO: 292 - DNA, 19 bp, Homo sapiens
ctggggatttcagcctctc

SEQ ID NO: 293 - DNA, 20 bp, Homo sapiens
aagagatggccccaaatagg

SEQ ID NO: 294 - DNA, 20 bp, Homo sapiens
acagagttgacggcgagagt

SEQ ID NO: 295 - DNA, 18 bp, Homo sapiens
agccaggatgccccctac

SEQ ID NO: 296 - DNA, 22 bp, Homo sapiens
gggtttaaacaccagcagaaag

SEQ ID NO: 297 - DNA, 18 bp, Homo sapiens
atcctgccctccctcatc

SEQ ID NO: 298 - DNA, 20 bp, Homo sapiens
atggctccaggaaggacatc

SEQ ID NO: 299 - DNA, 20 bp, Homo sapiens
ccaagacactcaaggtggtg

SEQ ID NO: 300 - DNA, 20 bp, Homo sapiens
ctgggaaagacaggcaacat

SEQ ID NO: 301 - DNA, 20 bp, Homo sapiens
ggccattctccctcttgttt

SEQ ID NO: 302 - DNA, 20 bp, Homo sapiens
agccactgctctcagcctaa

SEQ ID NO: 303 - DNA, 20 bp, Homo sapiens
ggacactatggcccagaaga

SEQ ID NO: 304 - DNA, 20 bp, Homo sapiens
gccactgctctcagcctaat

SEQ ID NO: 305 - DNA, 20 bp, Homo sapiens
ggaggctctgtctcaaaagc

SEQ ID NO: 306 - DNA, 20 bp, Homo sapiens
tcagcagtgtgaaaatcgac

SEQ ID NO: 307 - DNA, 23 bp, Homo sapiens
tcaaaagttctttgggacaaaac

SEQ ID NO: 308 - DNA, 22 bp, Homo sapiens
caattgtgttgagtttgcaatg